SEQUENCE LISTING (20 sequences)

SEQ ID NO: 1 - DNA, 20 bp, Artificial Sequence
  D19 PAM Sequence
acggaggctaagcgtcgcaa

SEQ ID NO: 2 - DNA, 25 bp, Artificial Sequence
  D19 Forward Primer
caccgacggaggctaagcgtcgcaa

SEQ ID NO: 3 - DNA, 25 bp, Artificial Sequence
  D19 Reverse Primer
ctgcctccgattcgcagcgttcaaa

SEQ ID NO: 4 - DNA, 20 bp, Artificial Sequence
  G6pd-1 PAM Sequence
catctcctccctgttccgtg

SEQ ID NO: 5 - DNA, 25 bp, Artificial Sequence
  G6pd-1 Forward Primer
caccgcatctcctccctgttccgtg

SEQ ID NO: 6 - DNA, 25 bp, Artificial Sequence
  G6pd-1 Reverse Primer
aaaccacggaacagggaggagatgc

SEQ ID NO: 7 - DNA, 23 bp, Artificial Sequence
  Ifng Forward Primer
tagctctgagacaatgaacgcta

SEQ ID NO: 8 - DNA, 23 bp, Artificial Sequence
  Ifng Reverse Primer
gtgattcaatgacgcttatgttg

SEQ ID NO: 9 - DNA, 19 bp, Artificial Sequence
  Gapdh Forward Primer
gccttccgtgttcctaccc

SEQ ID NO: 10 - DNA, 18 bp, Artificial Sequence
  Gapdh Reverse Primer
cagtgggccctcagatgc

SEQ ID NO: 11 - DNA, 24 bp, Artificial Sequence
  Rp18s Forward Primer
acctgtcttgataactgcccgtgt

SEQ ID NO: 12 - DNA, 24 bp, Artificial Sequence
  Rp18s Reverse Primer
taatggcagtgatggcgaaggcta

SEQ ID NO: 13 - DNA, 38 bp, Artificial Sequence
  Primer
agtcagcatatggtcagtcggatacacacatattcatc

SEQ ID NO: 14 - DNA, 33 bp, Artificial Sequence
  Primer
agtcagctcgagtcagagcttgtgggggttcac

SEQ ID NO: 15 - DNA, 20 bp, Artificial Sequence
  mPgd-1 PAM sequence
tcatgttcagaattaagttc

SEQ ID NO: 16 - DNA, 25 bp, Artificial Sequence
  mPgd-1 forward primer
caccgtcatgttcagaattaagttc

SEQ ID NO: 17 - DNA, 25 bp, Artificial Sequence
  mPgd-1 reverse primer
aaacgaacttaattctgaacatgac

SEQ ID NO: 18 - DNA, 20 bp, Artificial Sequence
  mPgd-2 PAM sequence
cggctttgtggtaagcggcg

SEQ ID NO: 19 - DNA, 25 bp, Artificial Sequence
  mPgd-2 forward primer
caccgcggctttgtggtaagcggcg

SEQ ID NO: 20 - DNA, 25 bp, Artificial Sequence
  mPgd-2 reverse primer
aaaccgccgcttaccacaaagccgc

(19) United States
(12) Patent Application Publication
(10) Pub. No.: US 2023/0348401 A1
(43) Pub. Date: Nov. 2, 2023

(54) Inhibitors of Glucose-6-phosphate Dehydrogenase and Uses Thereof

(71) Applicant: The Trustees of Princeton University, Princeton, NJ (US)

(72) Inventors: Jonathan M. Ghergurovich, Philadelphia, PA (US); Hahn Kim, Princeton, NJ (US); Joshua D. Rabinowitz, Princeton, NJ (US)

(21) Appl. No.: 17/758,847

(22) PCT Filed: Jan. 15, 2021
(86) PCT No.: PCT/US2021/013612

§ 371 Date: Jul. 14, 2022

Related U.S. Application Data
(60) Provisional application No. 62/961,491, filed on Jan. 15, 2020.

(51) Int. Cl. C07D 239/84; C07D 409/12; C07D 239/70; C07D 405/12; C07D 417/12; C07D 403/12; C07D 495/04; C07D 401/12; C07D 471/04; C07D 413/12; C07D 409/14; C07D 498/06

(52) U.S. Cl. CPC C07D 239/84; C07D 239/70; C07D 401/12; C07D 403/12; C07D 405/12; C07D 409/12; C07D 409/14; C07D 413/12; C07D 417/12; C07D 471/04; C07D 495/04; C07D 498/06

(57) ABSTRACT

Provided herein are compounds having the following structural formula, wherein values for the variables are as described herein. Also provided are pharmaceutical compositions of the compounds, as well as methods of using the compounds to inhibit the oxidative pentose phosphate pathway, e.g, to treat cancer, malaria, an autoimmune disease, an inflammatory condition or asthma.

### RELATED APPLICATION(S)

This application is the U.S. National Stage of International Application No. PCT/US2021/013612, filed Jan. 15, 2021, published in English, which claims the benefit of U.S. Provisional Application No. 62/961,491, filed on Jan. 15, 2020. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:

a) File name: 53911025002_Sequence_Listing.txt; created Aug. 5, 2022, 4,006 Bytes in size.

### GOVERNMENT SUPPORT

This invention was made with government support under Grant No. DK113643 awarded by the National Institutes of Health. The government has certain rights in the invention.

### BACKGROUND

Mammalian cells require the reduced cofactor NADPH for the biosynthesis of lipids, nucleotides and amino acids, for producing “oxidative burst” and for preserving cellular redox balance. To generate cytosolic NADPH, cells use malic enzyme (ME1), isocitrate dehydrogenase (IDH1) and/or the oxidative pentose phosphate pathway (oxPPP). The first and purportedly rate-limiting enzyme of the oxPPP, glucose-6-phosphate dehydrogenase (G6PD), has been extensively studied, in part due to the high occurrence of hypomorphic mutations in this X-linked gene. Furthermore, mounting evidence suggests that G6PD is upregulated in some pathologies, including certain cancers. Despite interest in this enzyme, no satisfactory compounds for altering G6PD activity in cells have been reported. For example, the steroid derivative dehydroepiandrosterone (DHEA), a widely cited inhibitor of G6PD, does not inhibit the oxPPP in cells.

Accordingly, there is a need for compounds that inhibit the oxPPP, for example, by inhibiting G6PD.

### SUMMARY

Provided herein is a compound of Structural Formula V:

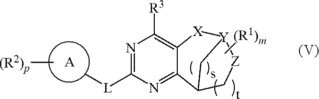

or a pharmaceutically acceptable salt thereof, wherein values for the variables (e.g., Ring A, L, X, Y, Z, R¹, R², R³, m, p, s, t) are as described herein.

Also provided herein is a composition comprising a compound described herein (e.g., a compound of any one of Structural Formulas I-VI), or a pharmaceutically acceptable salt thereof, and a pharmaceutically acceptable carrier.

Also provided herein is a method of treating a G6PD-mediated disease or condition in a subject in need thereof, comprising administering to the subject a therapeutically effective amount of a compound described herein (e.g., a compound of any one of Structural Formulas I-VI), or a pharmaceutically acceptable salt thereof.

Also provided herein is a method of treating a cancer in a subject in need thereof, comprising administering to the subject a therapeutically effective amount of a compound described herein (e.g., a compound of any one of Structural Formulas I-VI), or a pharmaceutically acceptable salt thereof.

Also provided herein is a method of treating malaria in a subject in need thereof, comprising administering to the subject a therapeutically effective amount of a compound described herein (e.g., a compound of any one of Structural Formulas I-VI), or a pharmaceutically acceptable salt thereof.

Also provided herein is a method of treating an autoimmune disease in a subject in need thereof, comprising administering to the subject a therapeutically effective amount of a compound described herein (e.g., a compound of any one of Structural Formulas I-VI), or a pharmaceutically acceptable salt thereof.

Also provided herein is a method of treating an inflammatory disease or condition, comprising administering to the subject a therapeutically effective amount of a compound described herein (e.g., a compound of any one of Structural Formulas I-VI), or a pharmaceutically acceptable salt thereof.

Also provided herein is a method of treating asthma, comprising administering to the subject a therapeutically effective amount of a compound described herein (e.g., a compound of any one of Structural Formulas I-VI), or a pharmaceutically acceptable salt thereof.

Also provided herein is a compound for use in the treatment of a disease or condition described herein (e.g., cancer, malaria, an autoimmune disease, an inflammatory disease or condition or asthma), wherein the compound is a compound described herein (e.g., a compound of any one of Structural Formulas I-VI), or a pharmaceutically acceptable salt thereof. Also provided herein is use of a compound described herein (e.g., a compound of any one of Structural Formulas I-VI), or a pharmaceutically acceptable salt thereof, for the manufacture of a medicament for the treatment of a disease or condition described herein (e.g., cancer, malaria, an autoimmune disease, an inflammatory disease or condition or asthma).

The compounds described herein (e.g., compounds of Structural Formulas I-VI) exhibit dose-dependent effects consistent with decreased cellular activity of G6PD, and can be used, e.g., as cancer therapeutics, prophylactic or acute treatments for malaria and/or immunomodulatory agents, and/or to modulate conditions associated with overactive inflammatory responses.

### BRIEF DESCRIPTION OF THE DRAWINGS

The foregoing will be apparent from the following more particular description of example embodiments, as illustrated in the accompanying drawings.

FIG. 1A shows the G6PD-diaphorase coupled fluorometric assay used for determining in vitro activity. G6P = glucose-6-phosphate, 6-pglac = 6-phosphogluconolactone.

FIG. 1B shows assays for G6PD cellular activity: (i) 6-phosphogluconate (6-pg) levels in Hep52 cells, (ii) deuterium (²H, small black circle) incorporation into NADPH (active hydride) and palmitic acid from 1-²H-glucose in HCT116 cells.

FIG. 1C is a graph of relative 6-pg levels, and shows DHEA (100 µM, 2 hours) does not phenocopy G6PD knockout (TIC, total ion count by LC-MS) (mean ± SD, n = 3).

FIG. 1D is a graph of relative NADPH active hydride labeling, and shows DHEA (100 µM, 2 hours) does not phenocopy G6PD knockout (TIC, total ion count by LC-MS) (mean ± SD, n = 3).

FIG. 1E shows that G6P labeling from 1-²H-glucose indicates that NADPH labeling differences in FIG. 1D are not due to changes in G6P labeling (mean ± SD, n = 4).

FIG. 1F is a graph of the C 16:0 (palmitic acid) ²H-labeled fraction, and shows DHEA (100 µM, 2 hours) does not phenocopy G6PD knockout (TIC, total ion count by LC-MS) (mean ± SD, n = 3).

FIG. 1G shows that CB-83 and polydatin (50 µM, 2 hours) do not decrease 6-pg levels (mean ± SD, n = 3).

FIG. 1H shows that CB-83 and polydatin (50 µM, 2 hours) do not decrease NADPH labeling (mean ± SD, n = 3).

FIG. 2A shows 6-pg production by purified G6PD is blocked by G6PDi-1 (LC-MS data, mean ± SD, n = 4).

FIG. 2B shows purified G6PD shows non-competitive inhibition by G6PDi-1 (RFU = relative fluorescence units, mean ± SD, n = 3).

FIG. 2C shows inhibition of purified G6PD by G6PDi is reversible. Data show activity relative to inhibitor-free controls, pre- (1x) and post-dilution (50x). [Compound 2] indicates final G6PDi-1 concentrations at listed dilutions (mean ± SD, n = 4).

FIG. 2D shows 6-pg dose-response curves obtained in HepG2 cells (mean ± SD, n = 3).

FIG. 2E shows rapid reversibility of the cellular activity of Compound 2. HepG2 cells were pre-treated with indicated media for two hours, followed by incubation with final media for two hours (mean ± SD, n = 3).

FIG. 2F shows NADPH active hydride ²H-labeling dose-response curves (HCT116 cells, 1-2H-glucose tracer) (mean ± SD, n = 3).

FIG. 2G shows free palmitic acid ²H-labeling dose-response curves (HCT116 cells, 1-2H-glucose tracer) (mean ± SD, n = 3).

FIG. 2H shows G6P labeling from 1-²H-glucose in HCT116 cells indicates that labeling differences in FIGS. 2F and 2G are not due to changes in G6P labeling (mean ± SD, n = 3).

FIG. 2I shows NADP+/NADPH ratio dose-response curves obtained in HCT116 cells (mean ± SD, n = 3).

FIG. 2J shows dUMP dose-response curves obtained in HCT116 cells (mean + SD, n = 3).

FIG. 3A shows LC-MS quantification of NADPH and NADP+ pools across a variety of normal and transformed cell types in response to G6PDi-1 (mean ± SD, n ≥ 3). TIC = total ion count. Cell names in red are T cell lineage.

FIG. 3B shows Western blots of G6PD, malic enzyme 1 (ME1) and isocitrate dehydrogenase 1 (IDH1) in the indicated cell lines.

FIG. 3C shows total oxPPP flux as determined by 14CO₂ emission in naïve mouse CD8+ T cells (cultured with IL-7) and activated mouse CD8+ T cells (day 4 post plate-bound αCD3/αCD28 and cultured with IL-2) (mean ± SD, n = 2).

FIG. 3D shows deuterium tracer strategies for quantifying NADPH sources.

FIG. 3E shows active hydride labeling of NADPH from tracers in listed cell types (mean ± SD, n = 3).

FIG. 3F shows fraction cellular NADPH from the oxPPP, ME1 and IDH1 in naïve and activated CD8+ T cells (for tracers, see FIGS. 3D and 3E).

FIG. 3G shows fraction cellular NADPH from the oxPPP, ME1 and IDH1 in naïve and activated CD4+ T cells (mean ± SD, n = 3).

FIG. 3H shows NADPH concentration and active hydride 2H-labeling dose response to G6PDi-1 (1-2H-glucose tracer) (mean ± SD, n = 3).

FIG. 3I shows NADPH concentration and active hydride 2H-labeling dose response to G6PDi-1 in active CD4+ cells (1-2H-glucose tracer) (mean ± SD, n = 3).

FIG. 3J shows 6-pg dose response in active CD8+ and CD4+ T cells (mean ± SD, n = 3).

FIG. 3K shows G6PDi-1 blocks oxPPP flux, as determined by ¹⁴CO₂ emission (mean ± SD, n = 5).

FIG. 3L shows NADP/NADPH shift in response to G6PDi-1 is rapidly reversible. Active CD8+ cells were pre-treated with indicated media for two hours, followed by incubation with final media for two hours (mean ± SD, n = 3).

FIG. 3M shows reversibility of G6PDi-1 effect on 6-pg levels in CD8+ T cells. Cells were pre-treated with indicated media for two hours, followed by incubation with final media for two hours (mean ± SD, n = 3).

FIG. 3N shows dynamics of NADPH, NADP+ and 6-pg pools, as well as active hydride labeling of NADPH from 1-2H-glucose in active CD8+ T cells in response to G6PDi-1 (50 µM) (mean ± SD, n = 3).

FIG. 30 shows absolute NADPH and NADP+ pools after G6PDi-1 (two hours) (mean ± SD, n = 3).

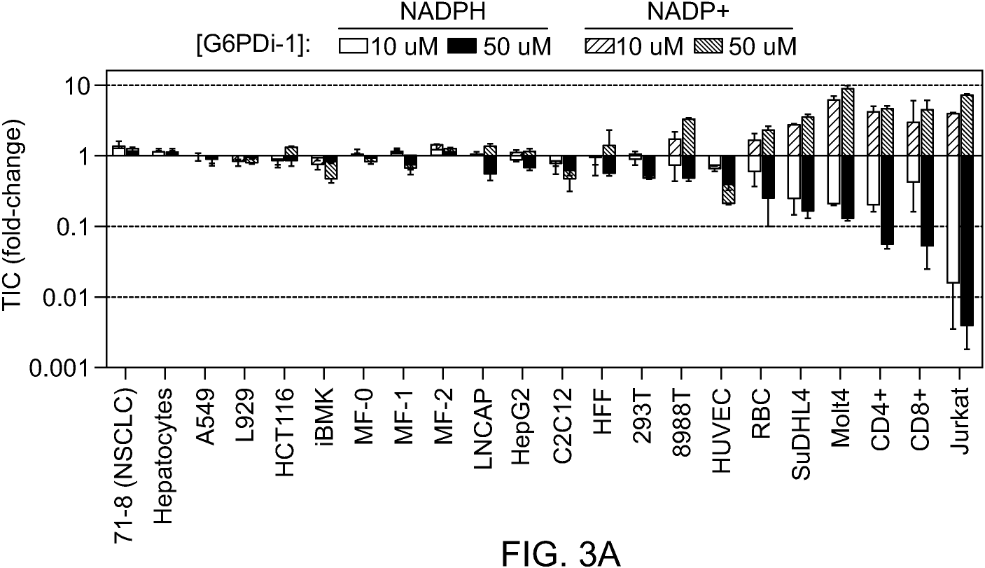

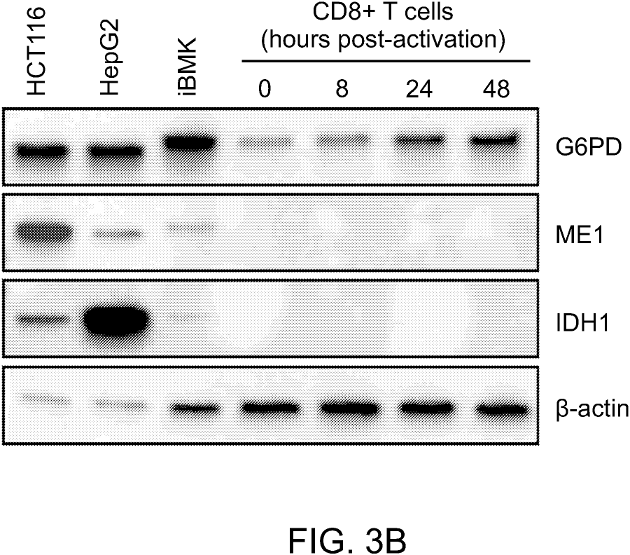

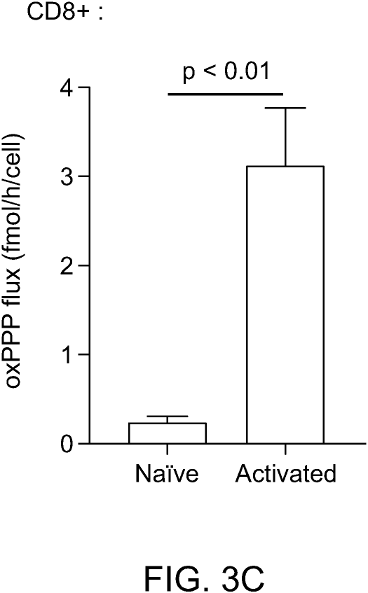

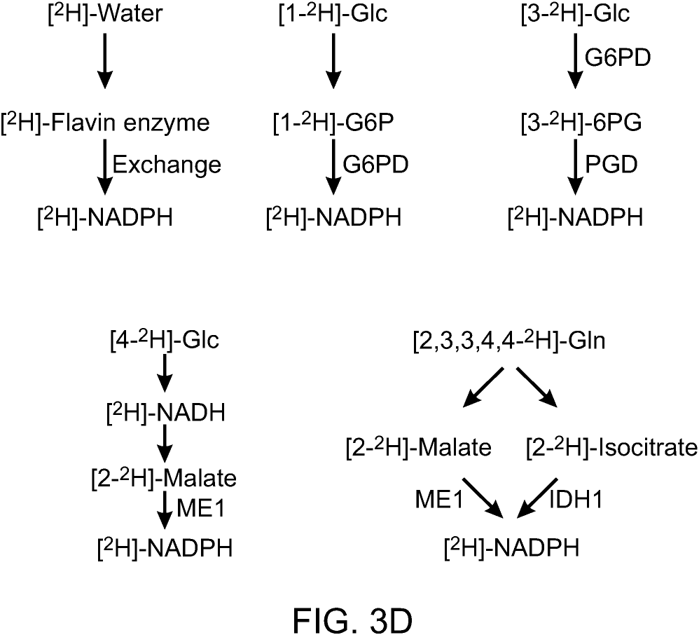

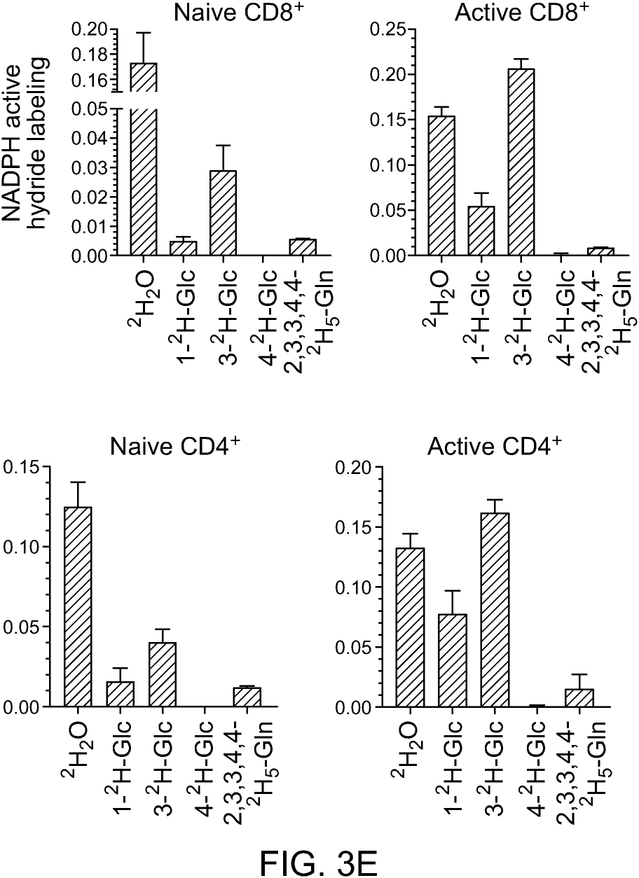

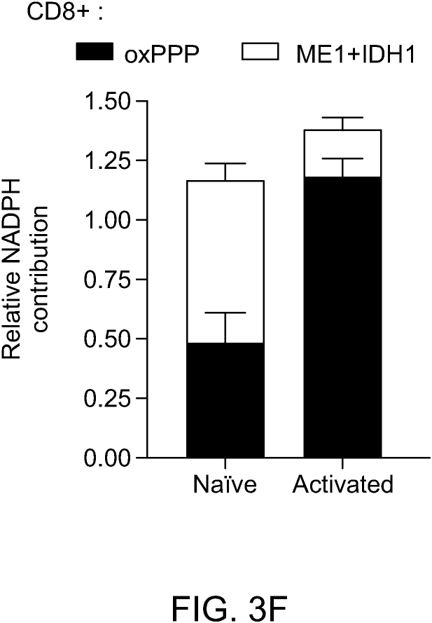

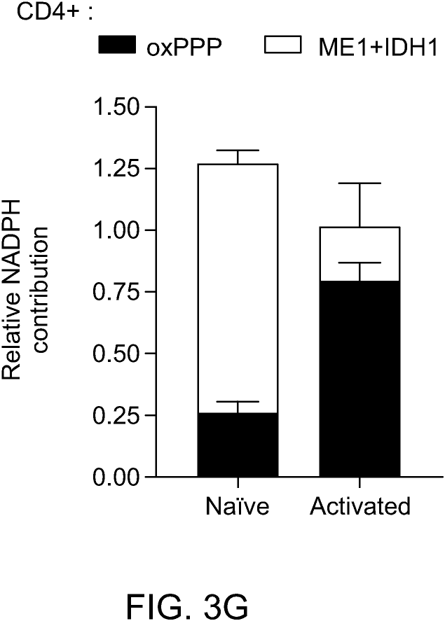

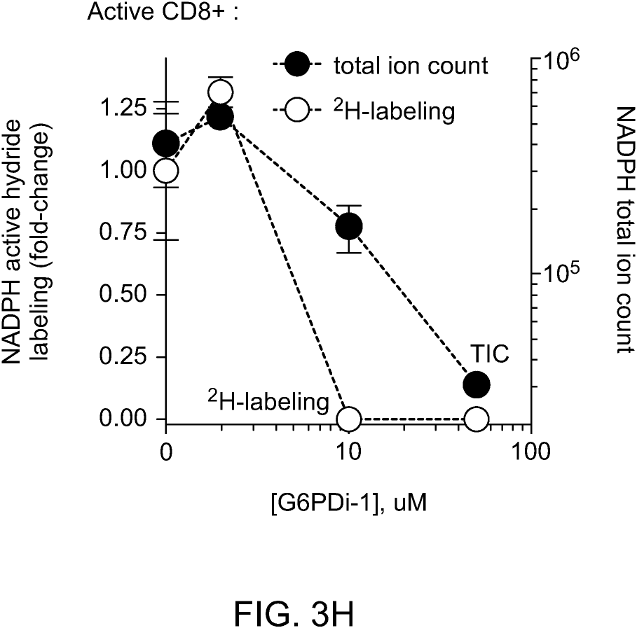

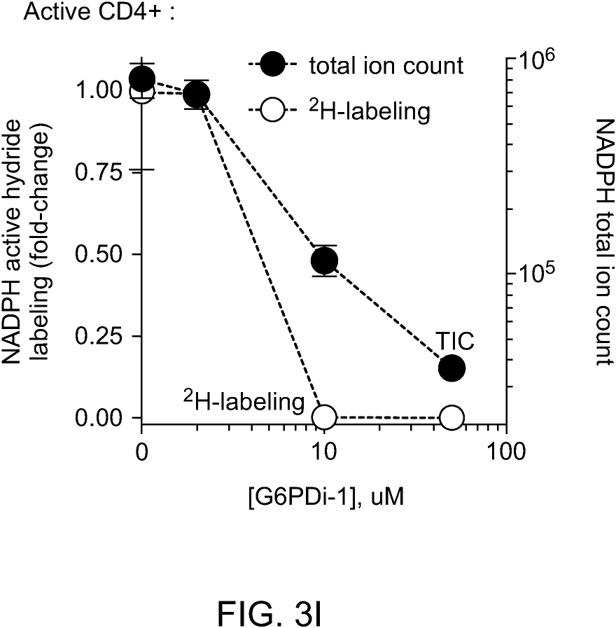

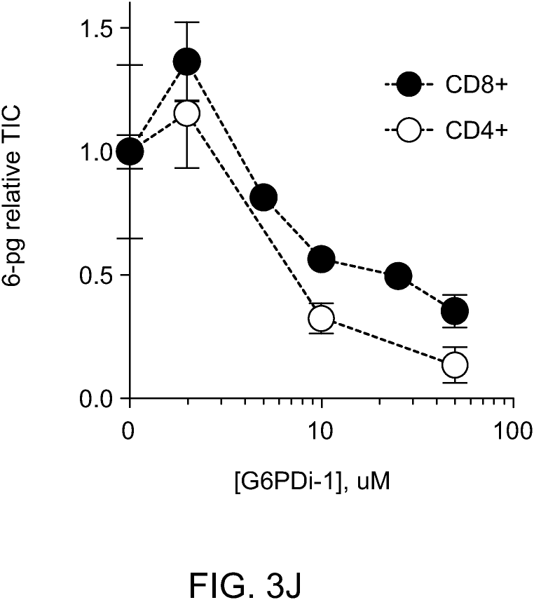

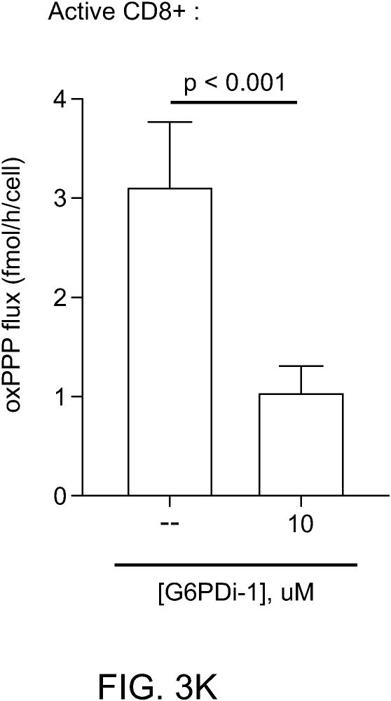

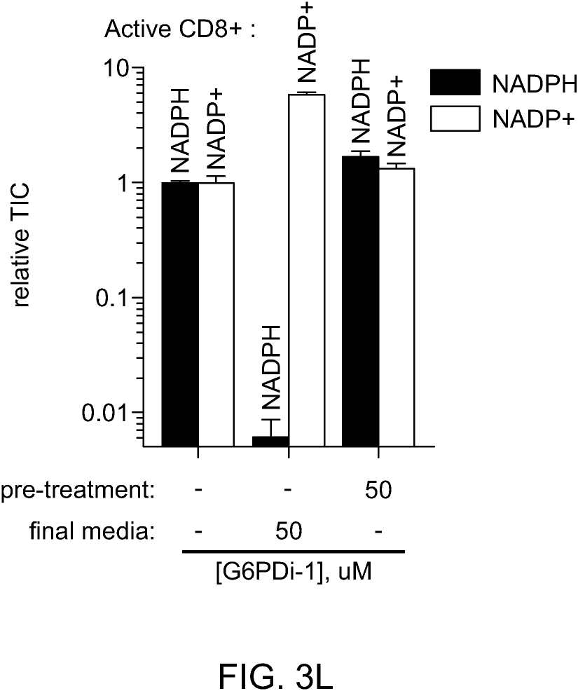

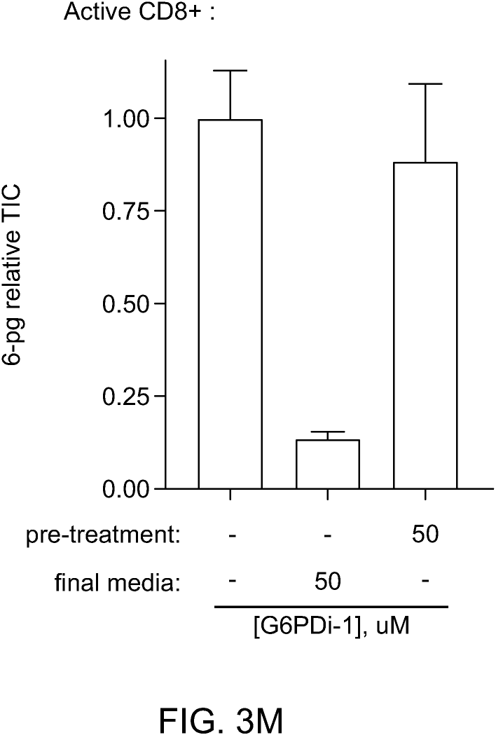

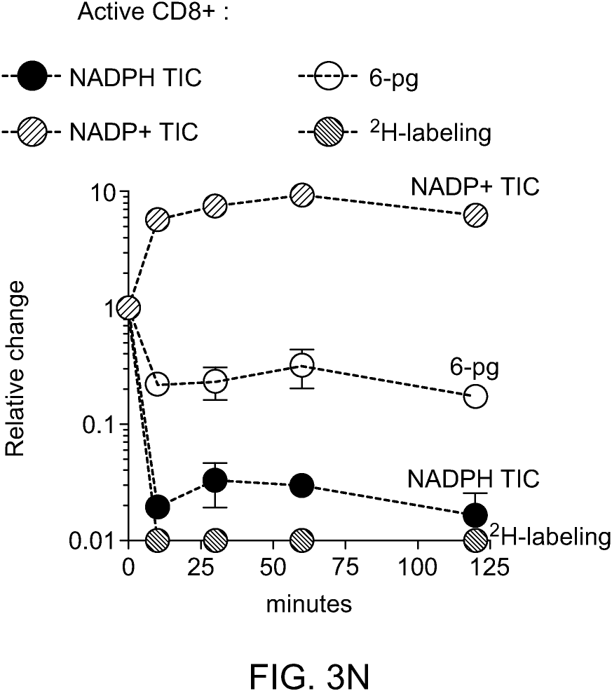

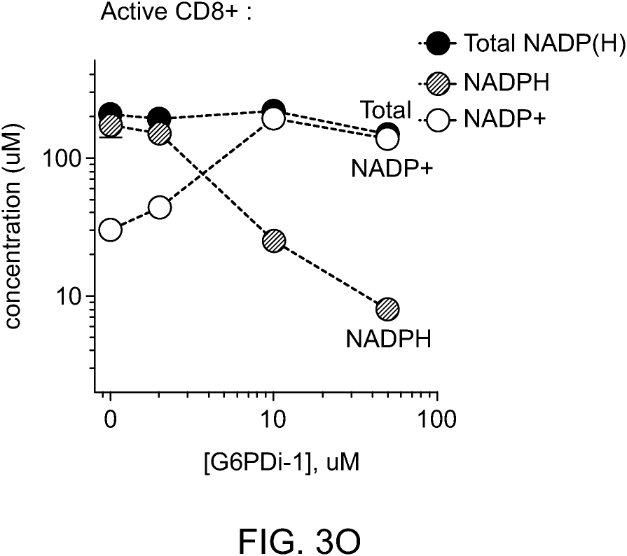

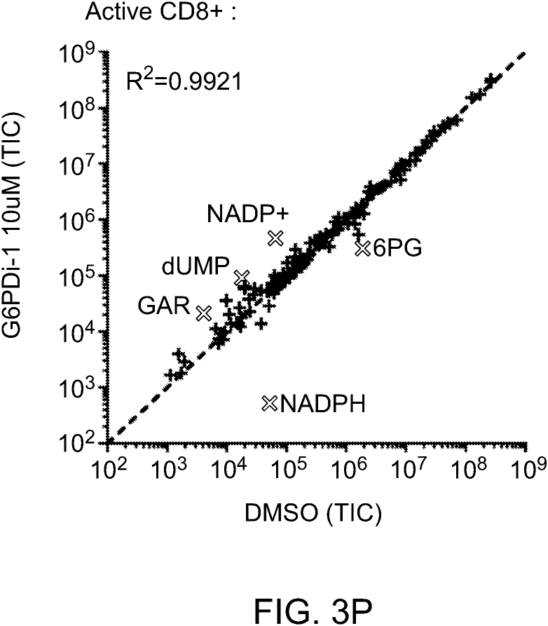

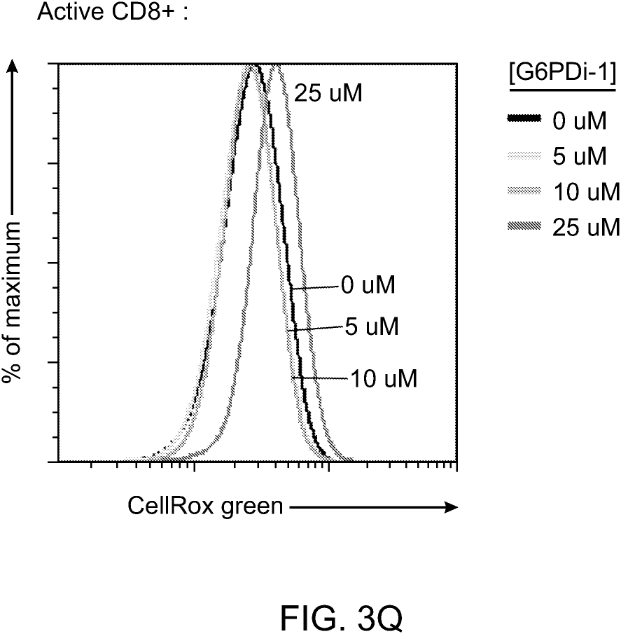

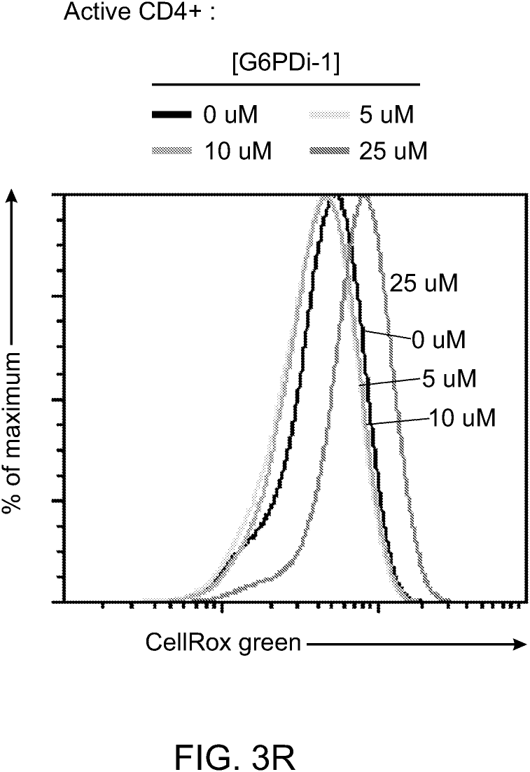

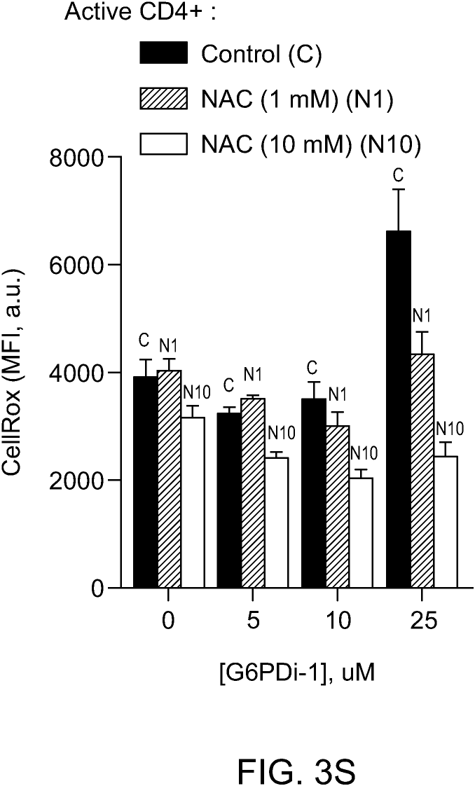

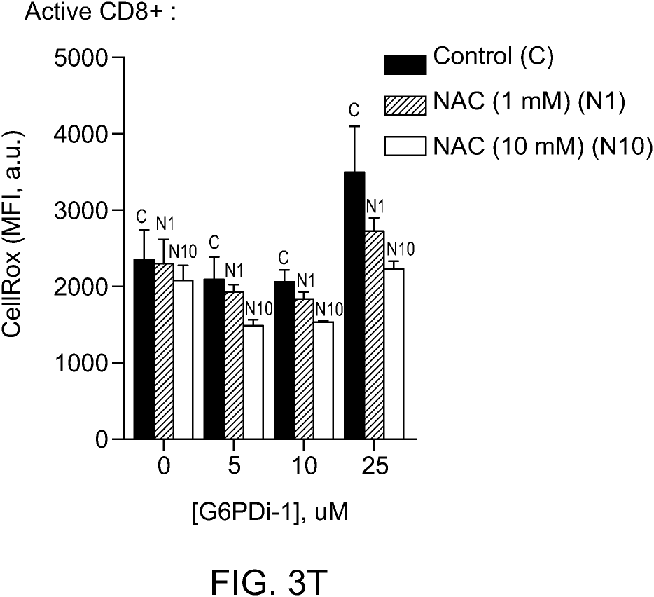

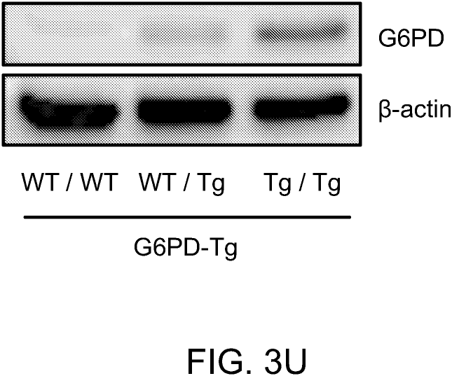

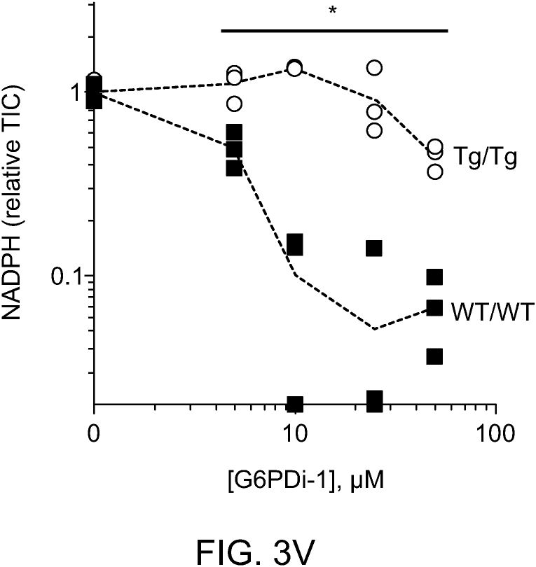

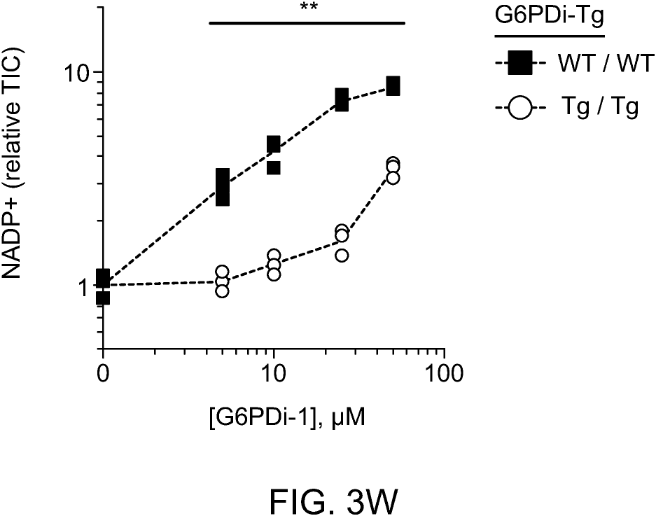

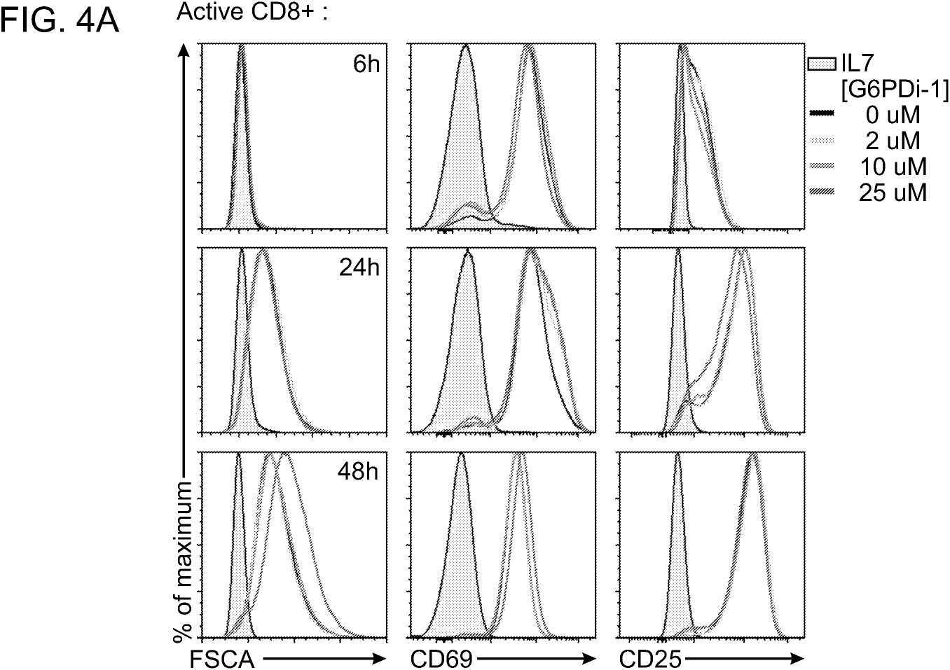

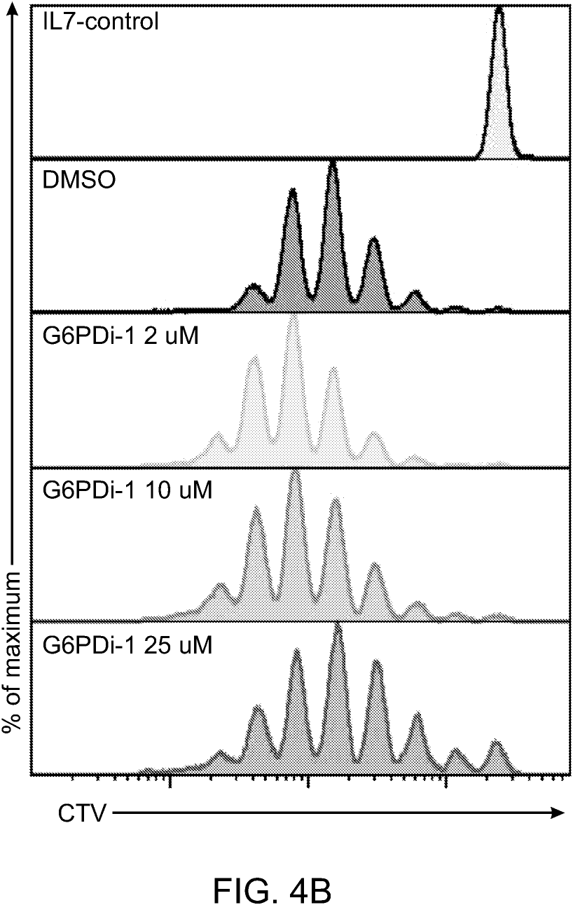

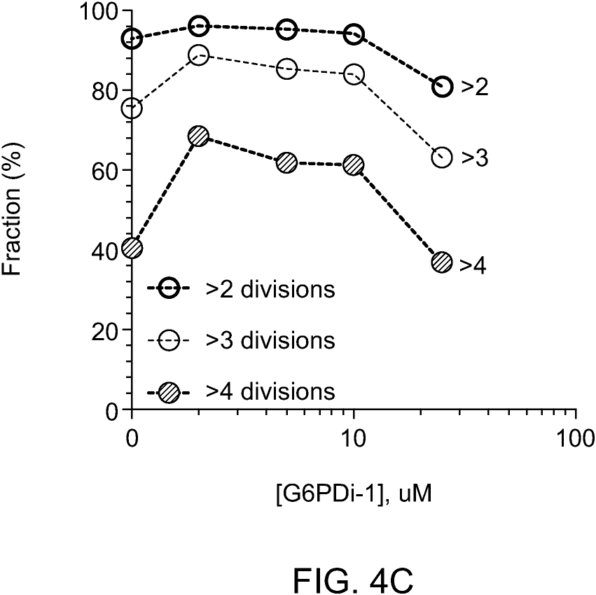

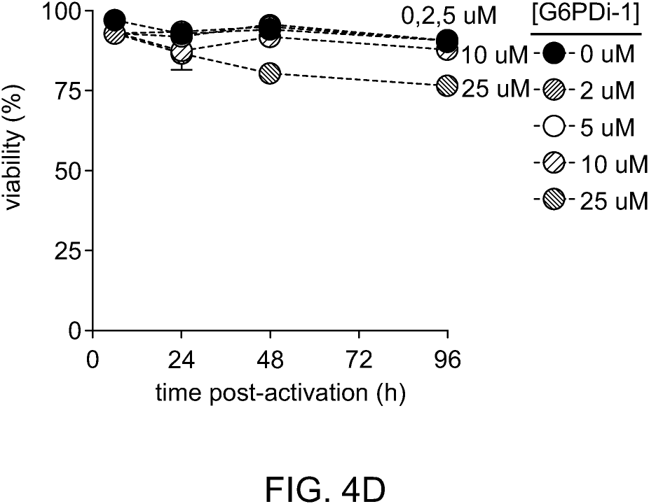

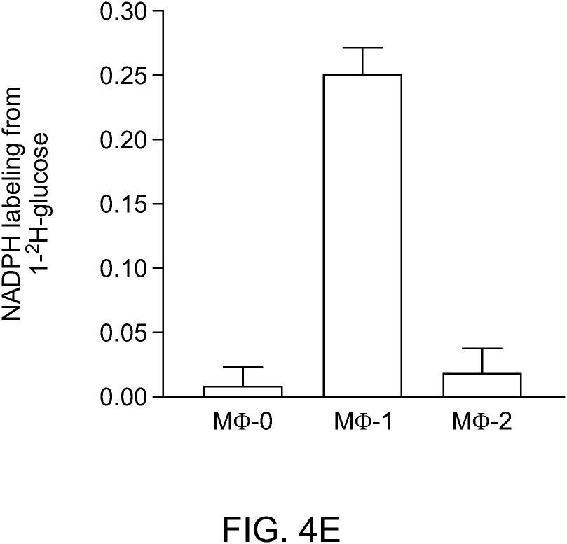

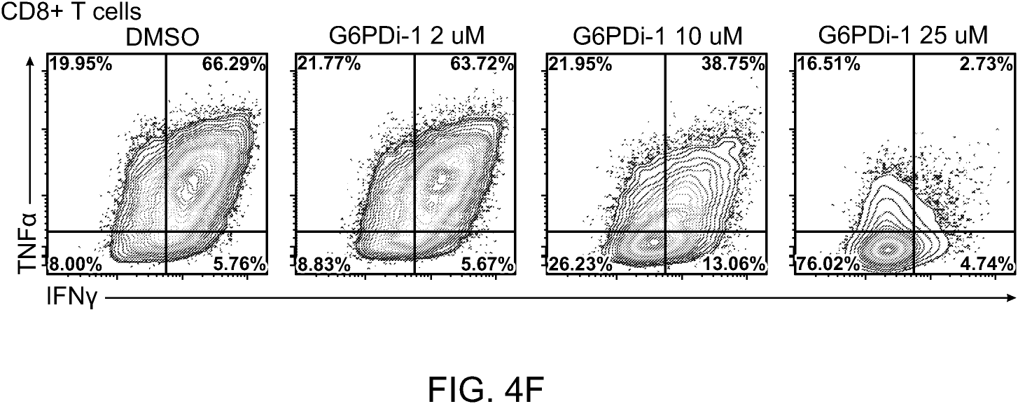

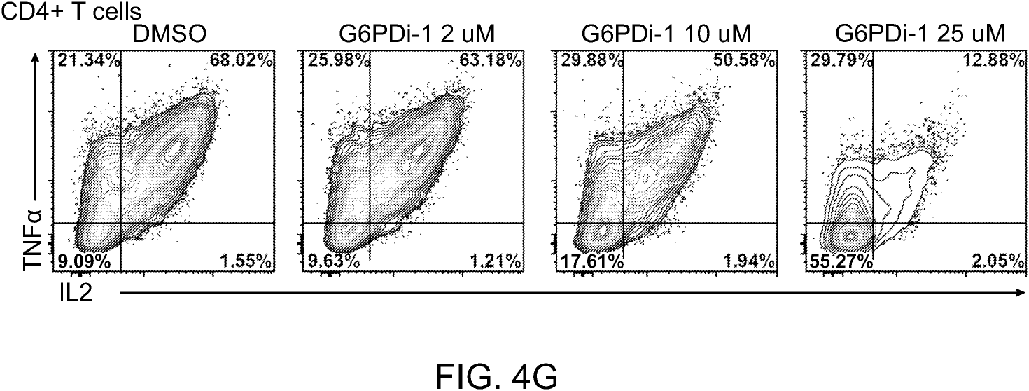

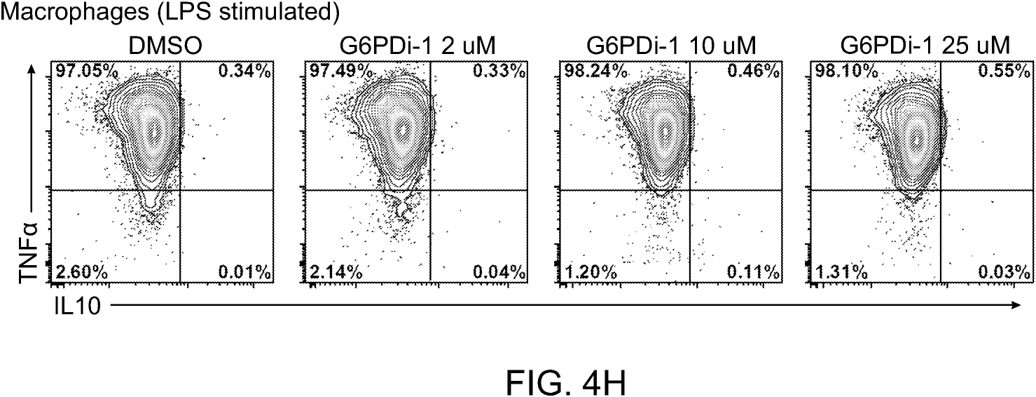

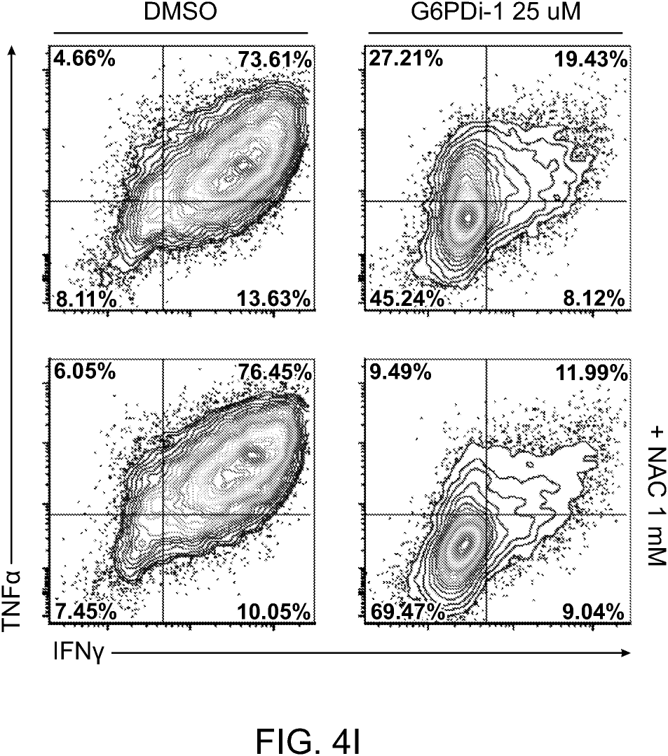

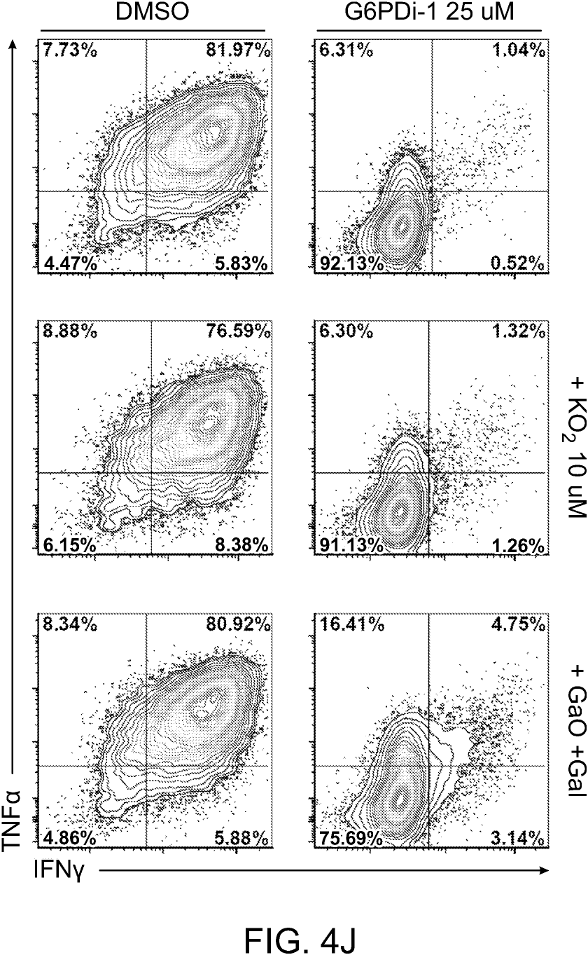

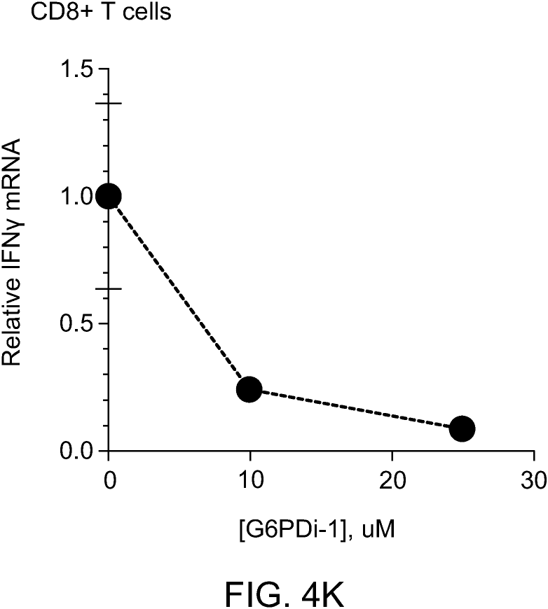

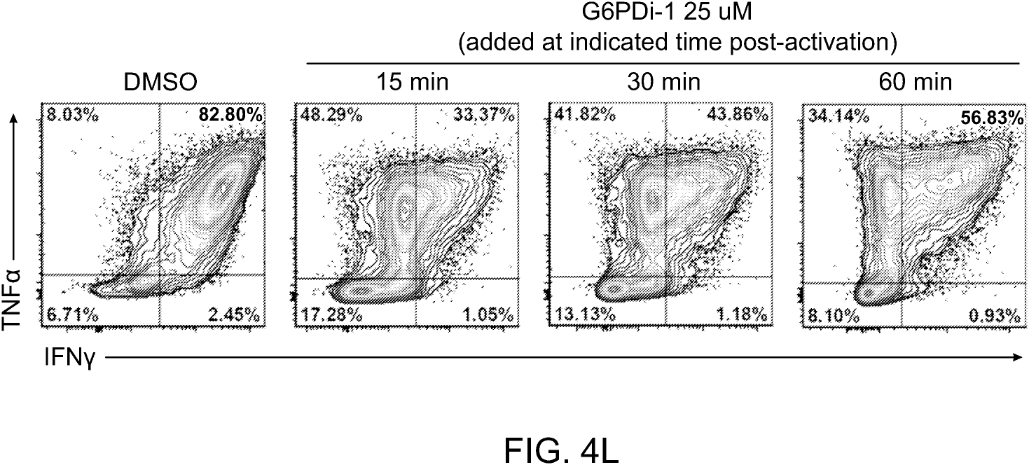

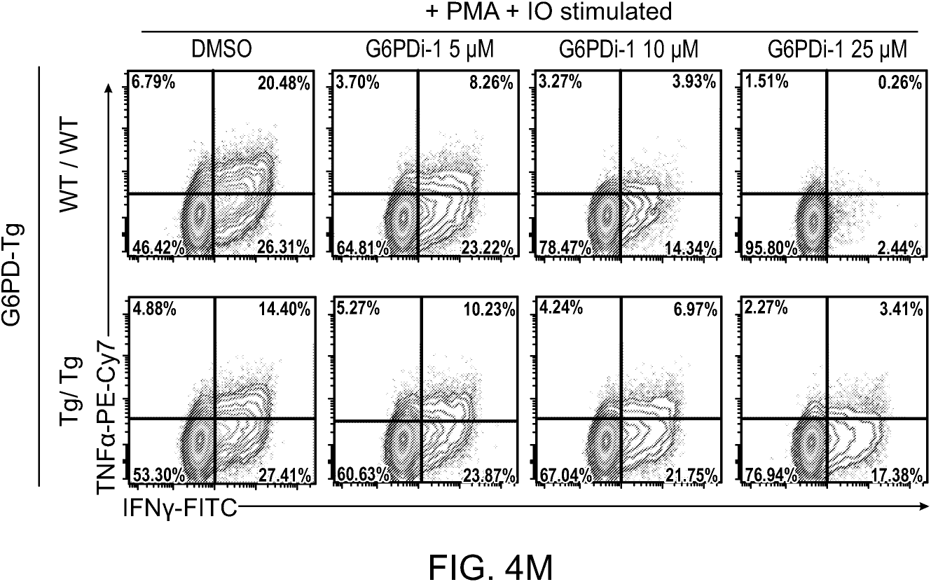

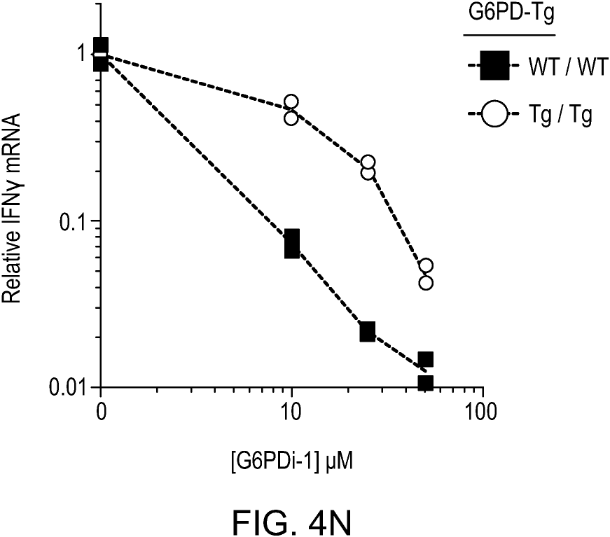

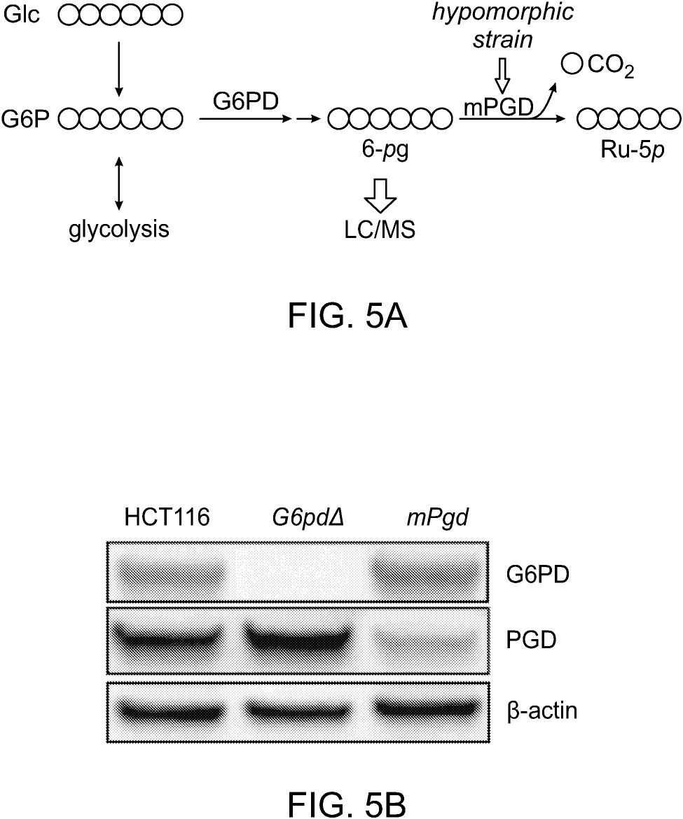

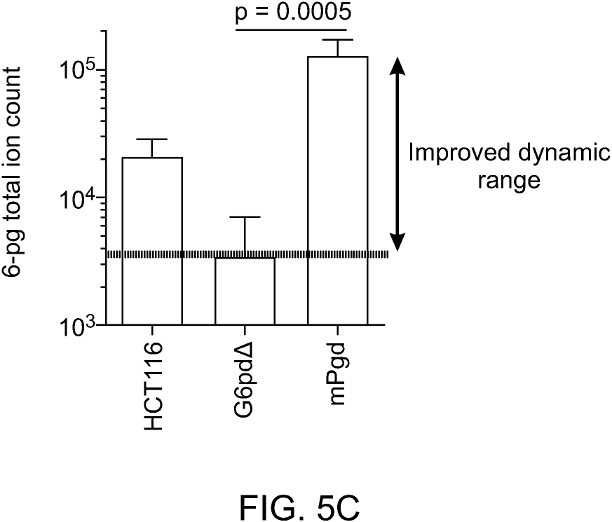

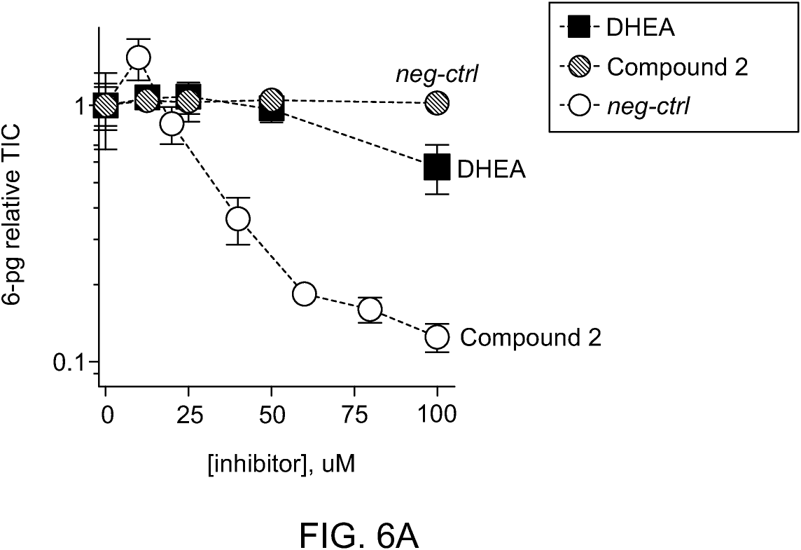

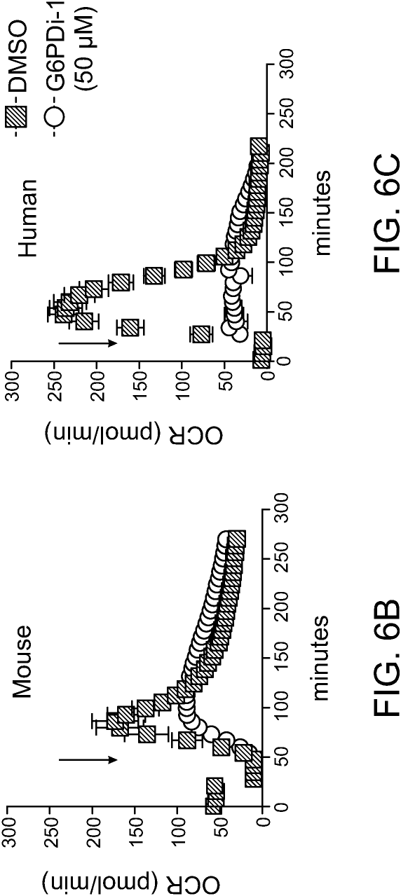

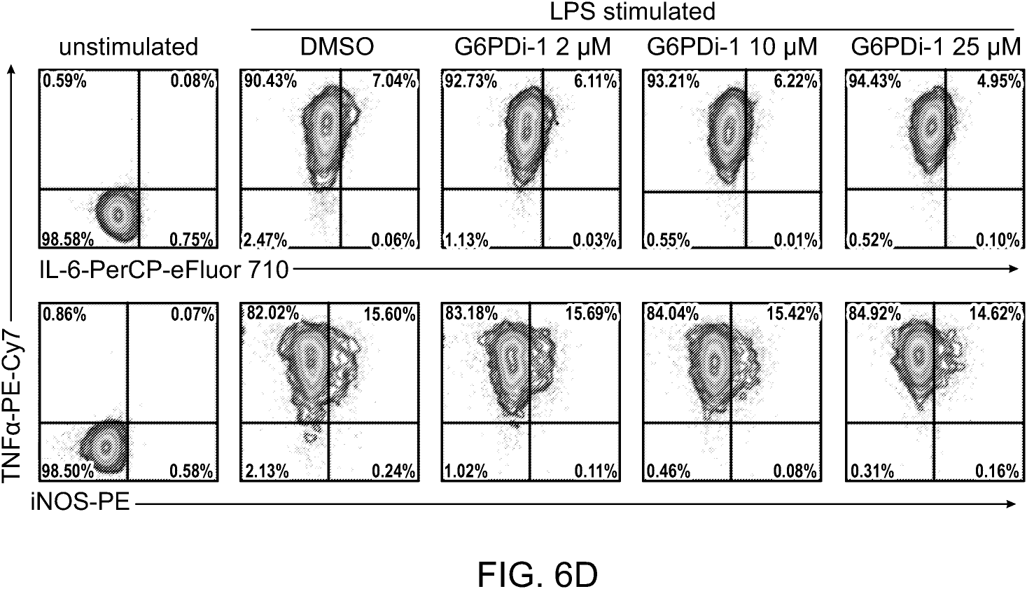

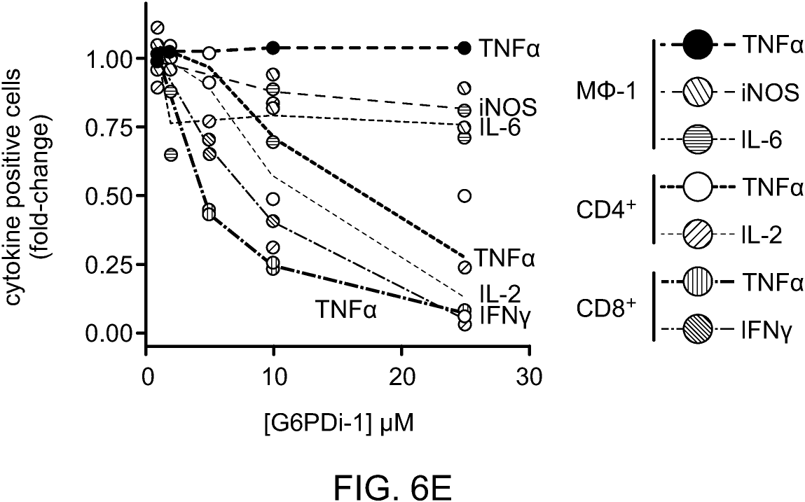

FIG. 3P shows water-soluble metabolite in active CD8+ T cells treated with G6PDi-1 (two hours) (mean ± SD, n = 3). Metabolites displaying a fold-change >4 are highlighted in red.

FIG. 3Q shows G6PDi-1 (two hours) induces ROS, as measured with Cell ROX green.

FIG. 3R shows G6PDi-1 (two hours) induces ROS, as measured with Cell ROX green, in active CD4+ T cells.

FIG. 3S shows ROS is suppressed by N-acetyl cysteine (NAC) (mean ± SD, n = 2). CD8+ T cells treated with G6PDi-1 (two hours) (mean ± SD, n = 3).

FIG. 3T shows ROS is suppressed by NAC (mean ± SD, n = 2).

FIG. 3U shows Western blots of G6PD (combined endogenous and transgenic) in active CD8⁺ T cells from G6pd overexpressing mice (G6PD-Tg mice). “WT / WT” = wild-type mice (no G6pd transgene expression); “WT / Tg” = heterozygous expression; “Tg / Tg” = homozygous expression. Representative results of 2 independent experiments.

FIG. 3V shows dose response to G6PDi-1 of NADPH in active CD8⁺ T cells from wild-type or G6pd overexpressing mice (n = 3). * and ** denote significant differences between WT/WT and Tg/Tg mice at each of the tested doses using a a two-tailed unpaired Student’s t-test. The following p values were obtained for NADPH levels: 5 µM, p = 0.011, 10 µM, p < 0.0001, 25 µM, p = 0.019, 50 µM, p = 0.0010. The following p values were obtained for NADP⁺ levels: 5 µM, p = 0.0011, 10 µM, p = 0.0012, 25 µM, p < 0.0001, 50 µM, p = < 0.0001.

FIG. 3W shows dose response to G6PDi-1 of NADP⁺ in active CD8⁺ T cells from wild-type or G6pd overexpressing mice (n = 3). * and ** denote significant differences between WT/WT and Tg/Tg mice at each of the tested doses using a a two-tailed unpaired Student’s t-test. The following p values were obtained for NADPH levels: 5 µM, p = 0.011, 10 µM, p < 0.0001, 25 µM, p = 0.019, 50 µM, p = 0.0010. The following p values were obtained for NADP⁺ levels: 5 µM, p = 0.0011, 10 µM, p = 0.0012, 25 µM, p < 0.0001, 50 µM, p = < 0.0001.

FIG. 4A are representative flow cytometry analyses of cell size (FSCA) and activation markers (CD69 and CD25) of mouse naïve CD8+ T cells either rested in IL-7 or stimulated by CD3/CD28 + IL-2 in the presence of increasing concentrations of G6PDi-1.

FIG. 4B shows proliferation after four days based on cell trace violet (CTV) dilution.

FIG. 4C shows representative flow cytometry analysis of quantification of cell divisions from FIG. 4B.

FIG. 4D show percent viability over time after treatment of CD8+ T cells with increasing concentrations of G6PDi-1.

FIG. 4E shows active hydride labeling of NADPH from 1-2H-glucose in subtypes of macrophages (mean ± SD, n = 3).

FIG. 4F shows a representative flow cytometry analysis of intracellular cytokines in the indicated mouse immune cells after a six-hour stimulation in the presence of the indicated dose of G6PDi-1. Stimulation with phorbol 12-myristate 13-acetate (PMA) and ionomycin.

FIG. 4G shows a representative flow cytometry analysis of intracellular cytokines in the indicated mouse immune cells after a six-hour stimulation in the presence of the indicated dose of G6PDi-1. Stimulation with PMA and ionomycin.

FIG. 4H shows a representative flow cytometry analysis of intracellular cytokines in the indicated mouse immune cells after a six-hour stimulation in the presence of the indicated dose of G6PDi-1. Stimulation with LPS and IFNγ for bone marrow-derived macrophages.

FIG. 4I shows a representative flow cytometry analysis of blockade of CD8+ T cell cytokine production by G6PDi-1 is not reversed by the ROS scavenger NAC.

FIG. 4J shows a representative flow cytometry analysis of blockade of CD8+ T cell cytokine production by G6PDi-1 is not reversed by the superoxide generators KO2 or GaO + Gal.

FIG. 4K shows corresponding Ifng mRNA in active CD8+ T cells (normalized to Gapdh expression and no G6PDi-1 control) (mean ± SD, n = 3).

FIG. 4L shows full blockage of cytokine secretion in active CD8+ T cells requires G6PDi-1 to be present over the initial hour of activation (six hour stimulation with PMA and ionomycin with G6PDi-1 added at the indicated times post the stimuli).

FIG. 4M shows intracellular cytokines in active CD8⁺ T cells from wild-type or G6pd overexpressing mice after a 6 h stimulation with PMA and IO in the presence of the indicated dose of G6PDi-1. Representative results of 2 independent experiments.

FIG. 4N shows corresponding Ifng mRNA in active CD8⁺ T cells from wild-type or G6pd overexpression mice (normalized to Gapdh expression and no G6PDi-1 control) (n = 2).

FIG. 5A is a schematic depicting direct monitoring of 6-pg by LC-MS in HCT116-mPgd cells.

FIG. 5B is an image of a Western blot comparing G6PD and PGD expression in clonal mPgd line, generated using CRISPR-Cas9.

FIG. 5C shows 6-pg total ion counts in HCT116, G6pdΔ, and mPgd lines (mean + SD, n = 4, one-way ANOVA).

FIG. 6A shows 6-pg dose-response curves in 6-phophogluconate hypomorphic HCT116 cells (HCT 116-mPgd cells) (mean ± SD, n = 3).

FIG. 6B is a graph of oxygen consumption rate (OCR) versus time, and shows the results of evaluation of neutrophil oxidative burst by the Seahorse Extracellular Flux Analyzer in mouse neutrophils that were activated with PMA (100 nM, indicated by arrow) and treated with or without G6PDi-1 (mean ± SEM, n = 6).

FIG. 6C is a graph of OCR versus time, and shows the results of evaluation of neutrophil oxidative burst by the Seahorse Extracellular Flux Analyzer in human neutrophils that were activated with PMA (100 nM, indicated by arrow) and treated with or without G6PDi-1 (mean ± SEM, n = 6).

FIG. 6D shows intracellular cytokines in bone marrow derived macrophages after a 6 h stimulation with LPS and IFNγ in the presence of the indicated dose of G6PDi-1. Representative results of 2 independent experiments.

FIG. 6E shows cytokine effects of G6PDi-1 across cell types (n = 2).

### DETAILED DESCRIPTION

A description of example embodiments follows.

### Definitions

Compounds described herein include those described generally, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March’s Advanced Organic Chemistry”, 5^{th} Ed., Ed.: Smith, M.B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

“Alkyl” refers to an optionally substituted, saturated, aliphatic, branched or straight-chain, monovalent, hydrocarbon radical having the specified number of carbon atoms. Thus, “(C₁-C₆)alkyl” means a radical having from 1-6 carbon atoms in a linear or branched arrangement. Examples of alkyl groups include methyl, ethyl, n-propyl, isopropyl, n-butyl, isobutyl, sec-butyl, t-butyl, n-pentyl, isopentyl, neopentyl, 2-methylpentyl, n-hexyl, and the like.

“Alkenyl” refers to an optionally substituted, aliphatic, branched or straight-chain, monovalent, hydrocarbon radical having at least one carbon-carbon double bond and the specified number of carbon atoms. Thus, “(C₁-C₆)alkenyl” means a radical having at least one carbon-carbon double bond and from 1-6 carbon atoms in a linear or branched arrangement. Examples of alkenyl groups include ethenyl, 2-propenyl, 1-propenyl, 2-methyl-1-propenyl, 1-butenyl, 2-butenyl, 1-pentenyl, 2-pentenyl, 3-pentenyl, allyl, 1, 3-butadienyl, 1, 3-dipentenyl, 1,4-dipentenyl, 1-hexenyl, 1,3-hexenyl, 1,4-hexenyl, 1,3,5-trihexenyl, 2,4-dihexenyl, and the like.

“Alkynyl” refers to an optionally substituted, aliphatic, branched or straight-chain, monovalent, hydrocarbon radical having at least one carbon-carbon triple bond and the specified number of carbon atoms. Thus, “(C₁-C₆)alkynyl” means a radical having at least one carbon-carbon triple bond and from 1-6 carbon atoms in a linear or branched arrangement. Examples of alkynyl groups include ethynyl, 1-propynyl, 2-propynyl, 1-butynyl, 2-butynyl, 2-methyl-1-butynyl, 1-pentynyl, 2-pentynyl, 3-pentynyl, 3-methyl-1-pentynyl, 2-methyl-1-pentynyl, 1-hexynyl, 2-hexynyl, 3-hexynyl, and the like.

“Aryl” refers to an optionally substituted, monocyclic or polycyclic (e.g., bicyclic, tricyclic), carbocyclic, aromatic ring system having the specified number of ring atoms, and includes aromatic rings fused to non-aromatic rings, as long as one of the fused rings is an aromatic hydrocarbon. Thus, “(C₆-C₁₅)aryl” means an aromatic ring system having from 6-15 ring atoms. Examples of aryl include phenyl and naphthyl.

“Heteroaryl” refers to an optionally substituted, monocyclic or polycyclic (e.g., bicyclic, tricyclic), aromatic, hydrocarbon ring system having the specified number of ring atoms, wherein at least one carbon atom in the ring system has been replaced with a heteroatom selected from N, S and O. “Heteroaryl” includes heteroaromatic rings fused to non-aromatic rings, as long as one of the fused rings is a heteroaromatic hydrocarbon. Thus, “(C₅-C₁₅)heteroaryl” means a heterocyclic aromatic ring system having from 5-15 ring atoms consisting of carbon, nitrogen, sulfur and oxygen. A heteroaryl can contain 1, 2, 3 or 4 (e.g., 1 or 2) heteroatoms independently selected from N, S and O. In one embodiment, heteroaryl has 5 or 6 ring atoms (e.g., five ring atoms). Monocyclic heteroaryls include, but are not limited to, furan, oxazole, thiophene, triazole, triazene, thiadiazole, oxadiazole, imidazole, isothiazole, isoxazole, pyrazole, pyridazine, pyridine, pyrazine, pyrimidine, pyrrole, tetrazole and thiazole. Bicyclic heteroaryls include, but are not limited to, indolizine, indole, isoindole, indazole, benzimidazole, benzofuran, benzothiazole, purine, quinoline, isoquinoline, cinnoline, phthalazine, quinazoline, quinoxaline, naphthyridine and pteridine.

“Cycloalkyl” refers to an optionally substituted, saturated, aliphatic, monovalent, monocyclic or polycyclic, hydrocarbon ring radical having the specified number of ring atoms. Thus, “(C₃-C₆)cycloalkyl” means a ring radical having from 3-6 ring carbons. Typically, cycloalkyl is monocyclic. Cycloalkyl includes, but is not limited to, cyclopropyl, cyclobutyl, cyclopentyl, cyclohexyl, and the like.

“Heterocyclyl” or “heterocycloalkyl” refers to an optionally substituted, saturated, aliphatic, monocyclic or polycyclic (e.g., bicyclic, tricyclic), monovalent, hydrocarbon ring system having the specified number of ring atoms, wherein at least one carbon atom in the ring system has been replaced with a heteroatom selected from N, S and O. Thus, “(C₃-C₆)heterocyclyl” means a heterocyclic ring system having from 3-6 ring atoms. A heterocyclyl can be monocyclic, fused bicyclic, bridged bicyclic or polycyclic, but is typically monocyclic. A heterocyclyl can contain 1, 2, 3 or 4 (e.g., 1) heteroatoms independently selected from N, S and O. When one heteroatom is S, it can be optionally mono- or di-oxygenated (i.e., —S(O)— or —S(O)₂). A heterocyclyl can be saturated (i.e., contain no degree of unsaturation). Examples of monocyclic heterocyclyls include, but are not limited to, aziridine, azetidine, pyrrolidine, piperidine, piperazine, azepane, tetrahydrofuran, tetrahydropyran, morpholine, thiomorpholine, dioxide, oxirane.

“Halogen” and “halo” are used interchangeably herein and each refers to fluorine, chlorine, bromine, or iodine. In some embodiments, halogen is selected from fluoro or chloro.

“Cyano” or “nitrile” means —CN.

“Hydroxy” means —OH.

“Hydroxyalkyl” refers to an alkyl radical wherein at least one hydrogen of the alkyl radical is replaced with hydroxy, and alkyl is as described herein. “Hydroxyalkyl” includes mono, poly, and perhydroxyalkyl groups.

“Alkoxy” refers to an alkyl radical attached through an oxygen linking atom, wherein alkyl is as described herein. Examples of alkoxy include, but are not limited to, methoxy, ethoxy, propoxy, isopropoxy, and the like.

“Aryloxy” refers to an aryl radical attached through an oxygen linking atom, wherein aryl is as described herein. Examples of aryloxy include, but are not limited to, phenoxy.

“Heteroaryloxy” refers to a heteroaryl radical attached through an oxygen linking atom, wherein heteroaryl is as described herein.

“Aralkoxy” refers to an aryl radical attached through an alkyl radical attached through an oxygen linking atom, wherein aryl and alkyl are as described herein. Examples of aralkoxy include benzyloxy.

“Heteroarylalkoxy” refers to a heteroaryl radical attached through an alkyl radical attached through an oxygen linking atom, wherein heteroaryl and alkyl are as described herein.

“Haloalkyl” includes mono, poly, and perhaloalkyl groups, wherein each halogen is independently selected from fluorine, chlorine, bromine and iodine (e.g., fluorine, chlorine and bromine), and alkyl is as described herein. In one aspect, haloalkyl is perhaloalkyl (e.g., perfluoroalkyl). Haloalkyl includes, but is not limited to, trifluoromethyl and pentafluoroethyl.

“Haloalkoxy” refers to a haloalkyl radical attached through an oxygen linking atom, wherein haloalkyl is as described herein.

It is understood that substituents on the compounds of the invention can be selected by one of ordinary skill in the art to provide compounds that are chemically stable and that can be readily synthesized by techniques known in the art, as well as those methods set forth below. The term “stable,” as used herein, refers to compounds that are not substantially altered when subjected to conditions to allow for their production, detection and, in certain embodiments, recovery, purification and use for one or more of the purposes disclosed herein.

Combinations of substituents envisioned by this invention are preferably those that result in the formation of stable or chemically feasible compounds.

A designated group is unsubstituted, unless otherwise indicated, e.g., by provision of a variable that denotes allowable substituents for a designated group. For example, R² in Structural Formula I denotes allowable substituents for Ring A. However, when the term “substituted,” whether preceded by the term “optionally” or not, precedes a designated group, it means that one or more hydrogens of the designated group are replaced with a suitable substituent. Unless otherwise indicated, an “optionally substituted” group or “substituted or unsubstituted” group can have a suitable substituent at each substitutable position of the group and, when more than one position in any given structure may be substituted with more than one substituent selected from a specified group, the substituent can be the same or different at every position. Alternatively, an “optionally substituted” group or “substituted or unsubstituted” group can be unsubstituted.

Suitable substituents include, but are not limited to, halo, hydroxy, cyano, —N(R¹⁴)C(O)N(R¹⁴)(R¹⁵), —N(R¹⁴)(R¹⁵), —C(O)N(R¹⁴)(R¹⁵), —C(O)OR¹⁴, (C1-C6)alkyl, (C1-C₆)alkenyl, (C₁-C₆)alkynyl, (C₁-C₆)haloalkyl, (C₁-C₆)hydroxyalkyl, (C₃-C₁₂)cycloalkyl, hydroxy(C₃-C₁₂)cycloalkyl, (C₃-C₁₂)heterocycloalkyl, (C₁-C₆)alkoxy, (C₁-C₆)haloalkoxy, (C₆-C₁₅)aryloxy, (C₅-C₁₅)heteroaryloxy, (C₆-C₁₅)ar(C₁-C₆)alkoxy, (C₅-C₁₅)heteroaryl(C₁-C₆)alkoxy, (C₆-C₁₅)aryl and (C₅-C₁₅)heteroaryl. In some embodiments, substituents are selected from halo, hydroxy, cyano, (C1-C6)alkyl, (C₁-C₆)haloalkyl, (C₁-C₆)alkoxy or (C₁-C₆)haloalkoxy.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethane sulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds described herein can also exist as various “solvates” or “hydrates.” A “hydrate” is a compound that exists in a composition with one or more water molecules. The composition can include water in stoichiometic quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

Compounds described herein can also exist as various solids, such as crystalline solids, e.g., polymorphs. Accordingly, also provided herein are polymorphic forms of the compounds described herein.

Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. In all provided structures, any hydrogen atom can also be independently selected from deuterium (²H), tritium (³H) and/or fluorine (F). Such compounds are useful, for example, as analytical tools, as probes in biological assays, or as therapeutic agents in accordance with the present invention.

Compounds disclosed herein may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, or as individual diastereomers or enantiomers, with all possible isomers and mixtures thereof, including optical isomers, being included in the present invention. When a disclosed compound is depicted by structure without indicating the stereochemistry, and the compound has one chiral center, it is to be understood that the structure encompasses one enantiomer or diastereomer of the compound separated or substantially separated from the corresponding optical isomer(s), a racemic mixture of the compound and mixtures enriched in one enantiomer or diastereomer relative to its corresponding optical isomer(s).

When a disclosed compound is depicted by a structure indicating stereochemistry, and the compound has more than one chiral center, the stereochemistry indicates relative stereochemistry, rather than the absolute configuration of the substituents around the one or more chiral carbon atoms. “R” and “S” are used to indicate the absolute configuration of substituents around one or more chiral carbon atoms.

“Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms.

“Racemate” or “racemic mixture,” as used herein, refer to a mixture containing equimolar quantities of two enantiomers of a compound. Such mixtures exhibit no optical activity (i.e., they do not rotate a plane of polarized light).

Percent enantiomeric excess (ee) is defined as the absolute difference between the mole fraction of each enantiomer multiplied by 100% and can be represented by the following equation:

$$\text{ee} \text{=} \left|\frac{\text{R} \text{−} \text{S}}{\text{R} \text{+} \text{S}}\right| \times 100 \% ,$$

where R and S represent the respective fractions of each enantiomer in a mixture, such that R + S = 1. An enantiomer may be present in an ee of at least or about 50%, about 60%, about 70%, about 80%, about 90%, about 95%, about 98%, about 99% or about 99.9%.

Percent diastereomeric excess (de) is defined as the absolute difference between the mole fraction of each diastereomer multiplied by 100% and can be represented by the following equation:

$$\text{de} = \left|\frac{\text{D1} \text{−} \left(\text{D2+D3+D4} ...\right)}{\text{D1} \text{+} \left(\text{D2+D3+D4} ...\right)}\right| \times 100 \% ,$$

where D1 and (D2 + D3 + D4...) represent the respective fractions of each diastereomer in a mixture, such that D1 + (D2 + D3 + D4...) = 1. A diastereomer may be present in a de of at least or about 50%, about 60%, about 70%, about 80%, about 90%, about 95%, about 98%, about 99% or about 99.9%.

In all provided structures, any stereocenter may specifically have D or L stereochemistry, or may be a racemic mixture.

When introducing elements disclosed herein, the articles “a,” “an,” “the,” and “said” are intended to mean that there are one or more of the elements. The terms “comprising,” “having” and “including” are intended to be open-ended and mean that there may be additional elements other than the listed elements.

### Compounds

A first embodiment is a compound of the following structural formula:

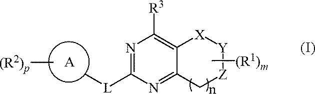

or a pharmaceutically acceptable salt thereof, wherein:
Ring A is (C₅-C₁₅)heteroaryl (e.g., thienyl) or (C₆-C₁₅)aryl (e.g., phenyl);
L is —N(R¹⁰)(C_{R}¹¹_{R}¹²)_{q}, —O—(CR¹¹R¹²)_{q}, —C(O)O—, —C(O)N(R¹⁰)—, —S(O)₂N(R¹⁰)—, —N(R¹⁰)C(O)N(R¹⁰)₋ or —C(R¹¹)(R ¹²)—;
   each R¹⁰, R¹¹ and R¹² is independently H or (C₁-C₆)alkyl;
   q is 0 or 1;
X is —C(O)—, —C(H)(OR¹³)—, —S(O)₂— or —C(NOR¹³)—;
   R₁₃ is H or (C₁-C₆)alkyl;
Y is —C(H)₂— or —N(H)—;
Z is —C(H)₂— or —O—;
R¹, for each occurrence, is independently halo, hydroxy, cyano, (C₁-C₆)alkyl, (C₁-C₆)haloalkyl, (C₁-C₆)alkoxy or (C₁-C₆)haloalkoxy;
R², for each occurrence, is halo, hydroxy, cyano, —N(R¹⁴)C(O)N(R¹⁴)(R¹⁵), —N(R14)(R¹⁵),
   —C(O)N(R¹⁴)(R¹⁵), —C(O)OR¹⁴, (C₁-C₆)alkyl, (C₁-C₆)alkenyl, (C₁-C₆)alkynyl, (C₁-C₆)haloalkyl, (C₁-C₆)hydroxyalkyl, (C₃-C₁₂)cycloalkyl, (C₃-C₁₂)heterocycloalkyl, (C₁-C₆)alkoxy, (C₁-C₆)haloalkoxy, (C₆-C₁₅)aryloxy, (C₅-C₁₅)heteroaryloxy, (C₆-C₁₅)ar(C₁-C₆)alkoxy, (C₅-C₁₅)heteroaryl(C₁-C₆)alkoxy, (C₆-C₁₅)aryl or (C₅-C₁₅)heteroaryl;
   each R¹⁴ and R¹⁵ is independently H or (C₁-C₆)alkyl;
R³ is H, halo, hydroxy, cyano, (C1-C6)alkyl, (C₁-C₆)haloalkyl, (C₁-C₆)alkoxy or (C₁-C₆)haloalkoxy;
n is 0, 1, 2 or 3 (e.g., 0, 1 or 2);
m is 0, 1, 2, 3 or 4; and
p is 0, 1, 2, 3 or 4.

In a first aspect of the first embodiment, when X is —C(O)—, Y is —C(H)₂—, Z is —C(H)₂— and n is 1, Ring A is not aryl (e.g., phenyl). Values for the remaining variables are as described in the first embodiment.

In a second aspect of the first embodiment, Ring A is (C₅-C₁₅)heteroaryl. Values for the remaining variables are as described in the first embodiment, or first aspect thereof.

In a third aspect of the first embodiment, Ring A is thienyl, pyrrolyl, pyridinyl, isoxazolyl, indazolyl, indolyl, benzofuranyl, benzthiazolyl or benzimidazolyl (e.g., thienyl, pyridinyl, isoxazolyl, indazolyl, indolyl, benzofuranyl, benzthiazolyl or benzimidazolyl). Values for the remaining variables are as described in the first embodiment, or first or second aspect thereof.

In a fourth aspect of the first embodiment, Ring A is thienyl (e.g., thien-3-yl, thien-4-yl). Values for the remaining variables are as described in the first embodiment, or first through third aspects thereof.

In a fifth aspect of the first embodiment, Ring A is (C₆-C₁₅)aryl (e.g., phenyl). Values for the remaining variables are as described in the first embodiment, or first through fourth aspects thereof.

In a sixth aspect of the first embodiment, L is —N(R¹⁰)(CR¹¹R¹²)_{q-}, —O—(CR¹¹R¹²)_{q-} or —C(O)N(R¹⁰)—. Values for the remaining variables are as described in the first embodiment, or first through fifth aspects thereof.

In a seventh aspect of the first embodiment, L is —N(H)—. Values for the remaining variables are as described in the first embodiment, or first through sixth aspects thereof.

In an eighth aspect of the first embodiment, each R¹⁰, R¹¹ and R¹² is independently H or methyl (e.g., H). Values for the remaining variables are as described in the first embodiment, or first through seventh aspects thereof.

In a ninth aspect of the first embodiment, q is 0. Values for the remaining variables are as described in the first embodiment, or first through eighth aspects thereof.

In a tenth aspect of the first embodiment, X is —C(O)— or —C(H)(OR¹³)—. Values for the remaining variables are as described in the first embodiment, or first through ninth aspects thereof.

In an eleventh aspect of the first embodiment, X is —C(O)—. Values for the remaining variables are as described in the first embodiment, or first through tenth aspects thereof.

In a twelfth aspect of the first embodiment, R¹³ is H. Values for the remaining variables are as described in the first embodiment, or first through eleventh aspects thereof.

In a thirteenth aspect of the first embodiment, Y is —C(H)₂—. Values for the remaining variables are as described in the first embodiment, or first through twelfth aspects thereof.

In a fourteenth aspect of the first embodiment, Z is —C(H)₂—. Values for the remaining variables are as described in the first embodiment, or first through thirteenth aspects thereof.

In a fifteenth aspect of the first embodiment, R², for each occurrence, is independently halo, hydroxy, cyano, —N(R¹⁴)C(O)N(R¹⁴)(R¹⁵), (C₁-C₆)alkyl, (C₁-C₆)alkenyl, (C₁-C₆)alkynyl, (C₁-C₆)haloalkyl, (C₁-C₆)hydroxyalkyl, (C₃-C₁₂)cycloalkyl, (C₃-C₁₂)heterocycloalkyl, (C₁-C₆)alkoxy, (C₁-C₆)haloalkoxy, (C₆-C₁₅)aryloxy, (C₅-C₁₅)heteroaryloxy, (C₆-C₁₅)ar(C₁-C₆)alkoxy or (C₅-C₁₅)heteroaryl(C₁-C₆)alkoxy. Values for the remaining variables are as described in the first embodiment, or first through fourteenth aspects thereof.

In a sixteenth aspect of the first embodiment, R², for each occurrence, is independently halo or cyano. Values for the remaining variables are as described in the first embodiment, or first through fifteenth aspects thereof.

In a seventeenth aspect of the first embodiment, R³ is H. Values for the remaining variables are as described in the first embodiment, or first through sixteenth aspects thereof.

In an eighteenth aspect of the first embodiment, n is 2. Values for the remaining variables are as described in the first embodiment, or first through seventeenth aspects thereof.

In a nineteenth aspect of the first embodiment, n is 1. Values for the remaining variables are as described in the first embodiment, or first through eighteenth aspects thereof.

In a twentieth aspect of the first embodiment, m is 0, 1 or 2. Values for the remaining variables are as described in the first embodiment, or first through nineteenth aspects thereof.

In a twenty-first aspect of the first embodiment, p is 0, 1 or 2 (e.g., 1 or 2). Values for the remaining variables are as described in the first embodiment, or first through twentieth aspects thereof.

In a twenty-second aspect of the first embodiment, p is 1. Values for the remaining variables are as described in the first embodiment, or first through twenty-first aspects thereof.

In a twenty-third aspect of the first embodiment, p is 2. Values for the remaining variables are as described in the first embodiment, or first through twenty-second aspects thereof.

In a twenty-fourth aspect of the first embodiment, p is 0. Values for the remaining variables are as described in the first embodiment, or first through twenty-third aspects thereof.

In a twenty-fifth aspect of the first embodiment, if p is 1, 2, 3 or 4 (e.g., 1 or 2), then one occurrence of R² is at the position meta to variable L. The term “meta,” used to describe the relationship between L and R² on Ring A, means that variables L and R² are in a 1,3-relationship to one another on Ring A, where the numerals 1 and 3 are used to describe the relationship between L and R², and do not necessarily correspond to the positions of L and R² on Ring A, for example, under IUPAC naming conventions. Thus, R² and L in Compound No. KG-0336-0 in Table 1 are in a 1,3-relationship to one another on Ring A, but are located at the 2- and 4-positions of the thienyl of Ring A. As also illustrated by Compound No. KG-0336-0, when R² is at the position meta to L, the ring atoms of Ring A to which L and R² are attached are themselves separated by one ring atom of Ring A. Many of the compounds in Table 1 wherein Ring A is substituted in addition to Compound No. KG-0336-0 are characterized by the occurrence of one R² at the position meta to variable L, e.g., KG-0206-0 and KG-0338. Values for the remaining variables (including Ring A and R²) are as described in the first embodiment, or first through twenty-fourth aspects thereof.

In a twenty-sixth aspect of the first embodiment, Ring A is phenyl. Values for the remaining variables are as described in the first embodiment, or first through twenty-fifth aspects thereof.

In a twenty-seventh aspect of the first embodiment, m is 0. Values for the remaining variables are as described in the first embodiment, or first through twenty-sixth aspects thereof.

In a twenty-eighth aspect of the first embodiment, Ring A is phenyl, thienyl or pyrrolyl. Values for the remaining variables are as described in the first embodiment, or first through twenty-seventh aspects thereof.

In a twenty-ninth aspect of the first embodiment, if p is 1, 2, 3 or 4 (e.g., 1 or 2), then each (e.g., 1 or 2) occurrence of R² is at the position meta to variable L. Values for the remaining variables (including Ring A and R²) are as described in the first embodiment, or first through twenty-eighth aspects thereof.

A second embodiment is a compound having the following structural formula:

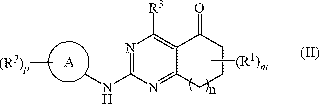

or a pharmaceutically acceptable salt thereof, wherein values for the variables (e.g., Ring A, R¹, R², R³, m, n, p) are as described in the first or fifth embodiment, or any aspect of the foregoing.

A third embodiment is a compound having the following structural formula:

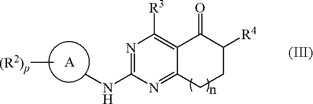

or a pharmaceutically acceptable salt thereof, wherein R⁴ is hydrogen, halo, hydroxy, cyano, (C₁-C₆)alkyl, (C₁-C₆)haloalkyl, (C₁-C₆)alkoxy or (C₁-C₆)haloalkoxy. Values for the remaining variables (e.g., Ring A, R², R³, n, p) are as described in the first or fifth embodiment, or any aspect of the foregoing.

In a first aspect of the third embodiment, R⁴ is hydrogen, halo, hydroxy, (C₁-C₆)alkyl or (C₁-C₆)haloalkyl. Values for the remaining variables are as described in the first or fifth embodiment, or any aspect of the foregoing, or the third embodiment.

In a second aspect of the third embodiment, Ring A is thienyl or phenyl. Values for the remaining variables are as described in the first or fifth embodiment, or any aspect of the foregoing, or the third embodiment, or first aspect thereof.

In a third aspect of the third embodiment, R², for each occurrence, is independently halo or cyano. Values for the remaining variables are as described in the first or fifth embodiment, or any aspect of the foregoing, or the third embodiment, or first or second aspect thereof.

A fourth embodiment is a compound having the following structural formula:

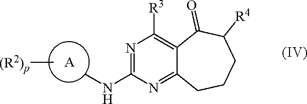

or a pharmaceutically acceptable salt thereof, wherein R⁴ is hydrogen, halo, hydroxy, cyano, (C₁-C₆)alkyl, (C₁-C₆)haloalkyl, (C₁-C₆)alkoxy or (C₁-C₆)haloalkoxy. Values for the remaining variables (e.g., Ring A, R², R³, p) are as described in the first, third or fifth embodiment, or any aspect of the foregoing.

A fifth embodiment is a compound of the following structural formula:

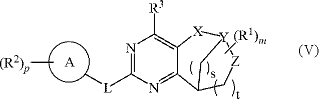

or a pharmaceutically acceptable salt thereof, wherein:
Ring A is (C₅-C₁₅)heteroaryl (e.g., thienyl, pyrrolyl) or (C₆-C₁₅)aryl (e.g., phenyl);
L is —N(R¹⁰)(CR¹¹R¹²)_{q}, —O—(CR¹¹R¹²)_{q}, —C(O)O—, —C(O)N(R¹⁰)—, —S(O)₂N(R¹⁰)—, ⁻N(R¹⁰)C(O)N(R¹⁰)— or —C(R¹¹)(R¹²)—;
   each R¹⁰, R¹¹ and R¹² is independently H or (C₁-C₆)alkyl;
   q is 0 or 1;
X is —C(O)—, —C(R¹⁶)—, —C(H)(OR¹³)—, —S(O)₂—, —C(NOR¹³)—, —C(F)₂—, —C(═C(CN)₂), —C(═C(H)(CN))— or —C(H)(C(H)(CN)₂)—;
   R¹³ is H or (C₁-C₆)alkyl;
Y is —C(H)₂—, —C(R¹⁷)— or —N(H)— when s is 0, and —C(H)— when s is 1 or 2;
   R¹⁶ and R¹⁷, taken together with X and Y, form a (C₅-C₆)heteroaryl optionally substituted with one, two or three substituents independently selected from halo, hydroxy, cyano, (C1-C6)alkyl, (C₁-C₆)haloalkyl, (C₁-C₆)alkoxy or (C₁-C₆)haloalkoxy;
Z is —C(H)₂— or —O—, or Z is absent;
R¹, for each occurrence, is independently halo, hydroxy, cyano, (C₁-C₆)alkyl, (C₁-C₆)haloalkyl, (C₁-C₆)alkoxy or (C₁-C₆)haloalkoxy;
R², for each occurrence, is independently halo, hydroxy, cyano, nitro, —N(R¹⁴)C(O)N(R¹⁴)(R¹⁵), -N(R¹⁴)(R¹⁵), —NR¹⁴C(O)R¹⁵, —S(O)₂N(R¹⁴)(R¹⁵), —S(O)₂R¹⁸,
   —CO)N(R¹⁴)(R¹⁵), —C(O)OR¹⁴, —C(O)R¹⁸, —OC(O)R¹⁸, (C₁-C₆)alkyl, (C₁-C₆)alkenyl, (C₁-C₆)alkynyl, (C₁-C₆)haloalkyl, (C₁-C₆)hydroxyalkyl, cyano(C₁-C₆)alkyl, (C₆-C₁₅)ar(C₁-C₆)alkyl, (C₃-C₁₂)cycloalkyl, (C₃-C₁₂)heterocycloalkyl, —OR¹⁸, —SR¹⁸, (C₆-C₁₅)aryl, (C₅-C₁₅)heteroaryl or B(OR¹⁹)₂;
   each R¹⁴ and R¹⁵ is independently H or (C₁-C₆)alkyl;
   each R¹⁸ is independently (C₁-C₁₅)alkyl, (C₁-C₁₅)alkenyl, (C₁-C₁₅)alkynyl, (C₁-C₁₅)haloalkyl, (C₁-C₁₅)hydroxyalkyl, (C₁-C₆)alkoxy(C₁-C₁₅)alkyl, amino(C₁-C₁₅)alkyl, (C₃-C₁₂)cycloalkyl, (C₃-C₁₂)heterocyclyl, (C₆-C₁₅)aryl, (C₅-C₁₅)heteroaryl, (C₃-C₁₂)cycloalkyl(C₁-C₁₅)alkyl, (C₃-C₁₂)heterocyclyl(C₁-C₁₅)alkyl, (C₆-C₁₅)ar(C₁-C₁₅)alkyl, (C₅-C₁₅)heteroaryl(C₁-C₁₅)alkyl, wherein each cycloalkyl, heterocyclyl, aryl and heteroaryl is optionally substituted with one or more (e.g., 1, 2, 3, 4, or 5; 1, 2 or 3; 1; 2; 3; 4; 5) R²⁰;
   each R¹⁹ is independently H or (C1-C6)alkyl, or two R¹⁹ attached to oxygens attached to the same B, taken together with their intervening atoms, form a (C₅-C_{g})heterocyclyl optionally and independently substituted with one or more (C₁-C₆)alkyl;
   each R²⁰ is independently halo, hydroxy, cyano, (C₁-C₆)alkyl, (C₁-C₆)haloalkyl, (C₁-C₆)hydroxyalkyl, —C(H)₂C≡C, (C₁-C₆)alkoxy, (C₁-C₆)haloalkoxy, —NH₂ or —C(O)O(C₁-C₆)alkyl;
R³ is H, halo, hydroxy, cyano, (C1-C6)alkyl, (C₁-C₆)haloalkyl, (C₁-C₆)alkoxy or (C₁-C₆)haloalkoxy;
m is 0, 1, 2, 3 or 4;
p is 0, 1, 2, 3 or 4;
s is 0, 1 or 2; and
t is 0, 1 or 2 (e.g., 0 or 1).

In a first aspect of the fifth embodiment, s is 0. Values for the remaining variables are as described in the first embodiment, or any aspect thereof, or fifth embodiment.

In a second aspect of the fifth embodiment, t is 1. Values for the remaining variables are as described in the first embodiment, or any aspect thereof, or fifth embodiment, or first aspect thereof.

In a third aspect of the fifth embodiment, the compound is represented by structural formula I, or a pharmaceutically acceptable salt thereof, wherein n is 0, 1, 2 or 3 (e.g., 0, 1 or 2; 1; 2). Values for the remaining variables are as described in the first embodiment, or any aspect thereof, or fifth embodiment, or first or second aspect thereof.

In a fourth aspect of the fifth embodiment, R², for each occurrence, is independently halo, hydroxy, cyano, (C₁-C₆)alkyl, (C₁-C₆)alkenyl, (C₁-C₆)alkynyl, (C₁-C₆)haloalkyl, —OR¹⁸, —SR¹⁸, (C₆-C₁₅)aryl or (C₅-C₁₅)heteroaryl (e.g., halo, hydroxy, cyano, (C1-C6)alkyl, (C₁-C₆)haloalkyl, —OR¹⁸ or (C₅-C₁₅)heteroaryl). Values for the remaining variables are as described in the first embodiment, or any aspect thereof, or fifth embodiment, or first through third aspects thereof.

In a fifth aspect of the fifth embodiment, each R¹⁸ is independently (C1-C6)alkyl, (C₁-C₆)alkenyl, (C₁-C₆)alkynyl, (C₁-C₆)haloalkyl, (C₁-C₆)hydroxyalkyl, (C₁-C₆)alkoxy(C₁-C₆)alkyl, amino(C₁-C₆)alkyl, (C₃-C₁₂)cycloalkyl, (C₃-C₁₂)heterocyclyl, (C₆-C₁₅)aryl, (C₅-C₁₅)heteroaryl, (C₃-C₁₂)cycloalkyl(C₁-C₆)alkyl, (C₃-C₁₂)heterocyclyl(C₁-C₆)alkyl, (C₆-C₁₅)ar(C₁-C₆)alkyl, (C₅-C₁₅)heteroaryl(C₁-C₆)alkyl, wherein each cycloalkyl, heterocyclyl, aryl and heteroaryl is optionally substituted with one or more (e.g., one) R²⁰. Values for the remaining variables are as described in the first embodiment, or any aspect thereof, or fifth embodiment, or first through fifth aspects thereof.

In a sixth aspect of the fifth embodiment, both R¹⁹ are H, both R¹⁹ are (C₁-C₆)alkyl, or two R¹⁹ attached to oxygens attached to the same B, taken together with their intervening atoms, form a (C₅-C₆)heterocyclyl optionally and independently substituted with one or more (e.g., 1, 2, 3 or 4; 1; 2; 3; 4) (C₁-C₆)alkyl. Values for the remaining variables are as described in the first embodiment, or any aspect thereof, or fifth embodiment, or first through fifth aspects thereof.

In a seventh aspect of the fifth embodiment, Ring A is phenyl; p is 2; and R², for one occurrence, is —OR¹⁸, and for a second occurrence, is selected from halo, hydroxy, cyano or (C₁-C₆)alkyl, wherein each R² is meta to variable L. Values for the remaining variables are as described in the first embodiment, or any aspect thereof, or fifth embodiment, or first through sixth aspects thereof.

In an eighth aspect of the fifth embodiment, each R¹⁸ is independently (C₁-C₆)alkyl, (C₁-C₆)alkenyl, (C₁-C₆)alkynyl, (C₁-C₆)haloalkyl, (C₃-C₁₂)cycloalkyl, (C₃-C₁₂)heterocyclyl, (C₆-C₁₅)aryl, (C₅-C₁₅)heteroaryl, (C₃-C₁₂)cycloalkyl(C₁)alkyl, (C₃-C₁₂)heterocyclyl(C₁)alkyl, (C₆-C₁₅)ar(C₁)alkyl, (C₅-C₁₅)heteroaryl(C₁)alkyl, wherein each cycloalkyl, heterocyclyl, aryl and heteroaryl is optionally substituted with one or more (e.g., one) R²⁰. Values for the remaining variables are as described in the first embodiment, or any aspect thereof, or fifth embodiment, or first through seventh aspects thereof.

In a ninth aspect of the fifth embodiment, each R²⁰ is independently halo, hydroxy, cyano, (C₁-C₆)alkyl, (C₁-C₆)haloalkyl, (C₁-C₆)hydroxyalkyl, —C(H)₂C≡C, (C₁-C₆)alkoxy or (C₁-C₆)haloalkoxy. Values for the remaining variables are as described in the first embodiment, or any aspect thereof, or fifth embodiment, or first through eighth aspects thereof.

In a tenth aspect of the fifth embodiment, X is —C(R¹⁶)—; Y is —C(R¹⁷)—; and R¹⁶ and R¹⁷, taken together with X and Y, form a (C₅-C₆)heteroaryl optionally substituted with one, two or three (e.g., one) substituents independently selected from halo, hydroxy, cyano, (C₁-C₆)alkyl, (C₁-C₆)haloalkyl, (C₁-C₆)alkoxy or (C₁-C₆)haloalkoxy. Values for the remaining variables are as described in the first embodiment, or any aspect thereof, or fifth embodiment, or first through ninth aspects thereof.

In an eleventh aspect of the fifth embodiment, the (C₅-C₆)heteroaryl formed by R¹⁶ and R¹⁷, taken together with X and Y, is an isoxazolyl, pyrazolyl, thienyl or pyridinyl optionally substituted with one, two or three (e.g., one) substituents independently selected from halo, hydroxy, cyano, (C₁-C₆)alkyl, (C₁-C₆)haloalkyl, (C₁-C₆)alkoxy or (C₁-C₆)haloalkoxy. Values for the remaining variables are as described in the first embodiment, or any aspect thereof, or fifth embodiment, or first through tenth aspects thereof.

In a twelfth aspect of the fifth embodiment, when (i) X is —C(O)—, Y is —C(H)₂—, Z is —C(H)₂— and t is 0; or (ii) X is —C(O)—, Y is —C(H)₂—, Z is absent and t is 1, then Ring A is not phenyl. Values for the remaining variables are as described in the first embodiment, or any aspect thereof, or fifth embodiment, or first through eleventh aspects thereof.

A sixth embodiment is a compound of the following structural formula:

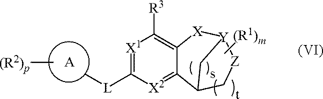

or a pharmaceutically acceptable salt thereof, wherein:
Ring A is (C₅-C₁₅)heteroaryl (e.g., thienyl, pyrrolyl) or (C₆-C₁₅)aryl (e.g., phenyl);
L is —N(R¹⁰)(CR¹¹R¹²)_{q}, —O—(CR¹¹R¹²)_{q}, —C(O)O—, —C(O)N(R¹⁰)—, —S(O)₂N(R¹⁰)—, —N(R¹⁰)C(O)N(R¹⁰)— or —C(R¹¹)(R¹²)—;
   each R¹⁰, R¹¹ and R¹² is independently H or (C₁-C₆)alkyl;
   q is 0 or 1;
X¹ is —N— or —C(R²¹)—;
   R²¹ is H, halo, hydroxy, cyano, (C₁-C₆)alkyl, (C₁-C₆)haloalkyl, (C₁-C₆)alkoxy or (C₁-C₆)haloalkoxy (e.g., H);
X² is —N— or —C(R²²)—;
   R²² is H, halo, hydroxy, cyano, (C₁-C₆)alkyl, (C₁-C₆)haloalkyl, (C₁-C₆)alkoxy or (C₁-C₆)haloalkoxy (e.g., H);
X is —C(O)—, —C(R¹⁶)—, —C(H)(OR¹³)—, —S(O)₂—, —C(NOR¹³)—, —C(F)₂—, —C(═C(CN)₂), —C(═C(H)(CN))— or —C(H)(C(H)(CN)₂)—;
   R₁₃ is H or (C₁-C₆)alkyl;
Y is —C(H)₂—, —C(R¹⁷)— or —N(H)— when s is 0, and —C(H)— when s is 1 or 2;
   R¹⁶ and R¹⁷, taken together with X and Y, form a (C₅-C₆)heteroaryl optionally substituted with one, two or three substituents independently selected from halo, hydroxy, cyano, (C₁-C₆)alkyl, (C₁-C₆)haloalkyl, (C₁-C₆)alkoxy or (C₁-C₆)haloalkoxy;
Z is —C(H)₂— or —O—, or Z is absent;
R¹, for each occurrence, is independently halo, hydroxy, cyano, (C₁-C₆)alkyl, (C₁-C₆)haloalkyl, (C₁-C₆)alkoxy or (C₁-C₆)haloalkoxy;
R², for each occurrence, is independently halo, hydroxy, cyano, nitro, —N(R¹⁴)C(O)N(R¹⁴)(R¹⁵), —N(R¹⁴)(R¹⁵), —NR¹⁴C(O)R¹⁵, —S(O)₂N(R¹⁴)(R¹⁵), —S(O)₂R¹⁸,
   —C(O)N(R¹⁴)(R¹⁵), —C(O)OR¹⁴, —C(O)R¹⁸, —OC(O)R¹⁸, (C₁-C₆)alkyl, (C₁-C₆)alkenyl, (C₁-C₆)alkynyl, (C₁-C₆)haloalkyl, (C₁-C₆)hydroxyalkyl, cyano(C₁-C₆)alkyl, (C₆-C₁₅)ar(C₁-C₆)alkyl, (C₃-C₁₂)cycloalkyl, (C₃-C₁₂)heterocycloalkyl, —OR¹⁸, —SR¹⁸, (C₆-C₁₅)aryl, (C₅-C₁₅)heteroaryl or B(OR¹⁹)₂;
   each R¹⁴ and R¹⁵ is independently H or (C₁-C₆)alkyl;
   each R¹⁸ is independently (C₁-C₁₅)alkyl, (C₁-C₁₅)alkenyl, (C₁-C₁₅)alkynyl, (C₁-C₁₅)haloalkyl, (C₁-C₁₅)hydroxyalkyl, (C₁-C₆)alkoxy(C₁-C₁₅)alkyl, amino(C₁-C₁₅)alkyl, (C₃-C₁₂)cycloalkyl, (C₃-C₁₂)heterocyclyl, (C₆-C₁₅)aryl, (C₅-C₁₅)heteroaryl, (C₃-C₁₂)cycloalkyl(C₁-C₁₅)alkyl, (C₃-C₁₂)heterocyclyl(C₁-C₁₅)alkyl, (C₆-C₁₅)ar(C₁-C₁₅)alkyl, (C₅-C₁₅)heteroaryl(C₁-C₁₅)alkyl, wherein each cycloalkyl, heterocyclyl, aryl and heteroaryl is optionally substituted with one or more (e.g., 1, 2, 3, 4, or 5; 1, 2 or 3; 1; 2; 3; 4; 5) R²⁰;
   each R¹⁹ is independently H or (C₁-C₆)alkyl, or two R¹⁹ attached to oxygens attached to the same B, taken together with their intervening atoms, form a (C₅-C₈)heterocyclyl optionally and independently substituted with one or more (C₁-C₆)alkyl;
   each R²⁰ is independently halo, hydroxy, cyano, (C₁-C₆)alkyl, (C₁-C₆)haloalkyl, (C₁-C₆)hydroxyalkyl, —C(H)₂C≡C, (C₁-C₆)alkoxy, (C₁-C₆)haloalkoxy, —NH₂ or —C(O)O(C₁-C₆)alkyl;
R³ is H, halo, hydroxy, cyano, (C₁-C₆)alkyl, (C₁-C₆)haloalkyl, (C₁-C₆)alkoxy or (C₁-C₆)haloalkoxy;
m is 0, 1, 2, 3 or 4;
p is 0, 1, 2, 3 or 4;
s is 0, 1 or 2; and
t is 0, 1 or 2 (e.g., 0 or 1).
Alternative values for the variables in structural formula VI are as described in the first or fifth embodiment, or any aspect of the foregoing.

In a first aspect of the sixth embodiment, X¹ is —N— and X² is —N—. Values for the remaining variables are as described in the first or fifth embodiment, or any aspect thereof, or sixth embodiment.

In a second aspect of the sixth embodiment, X¹ is —C(R²¹)— and X² is —C(R²²)—. Values for the remaining variables are as described in the first or fifth embodiment, or any aspect thereof, or sixth embodiment, or first aspect thereof.

In a third aspect of the sixth embodiment, X¹ is —C(R²¹)— and X² is —N—. Values for the remaining variables are as described in the first or fifth embodiment, or any aspect thereof, or sixth embodiment, or first or second aspect thereof.

In a fourth aspect of the sixth embodiment, X¹ is —N— and X² is —C(R²²)—. Values for the remaining variables are as described in the first or fifth embodiment, or any aspect thereof, or sixth embodiment, or first through third aspects thereof.

In a fifth aspect of the sixth embodiment, when X¹ and X² are both —N—; X is —C(O)—; Y is —C(H)₂—; and (i) Z is —C(H)₂— and t is 0, or (ii) Z is absent and t is 1, then Ring A is not phenyl. Values for the remaining variables are as described in the first or fifth embodiment, or any aspect thereof, or sixth embodiment, or first through fourth aspects thereof.

Tables 1 and 1A list representative compounds of the structural formulas depicted herein, and the normalized in vitro IC₅₀ designations associated with each, obtained using the diaphorase-coupled biochemical assay described herein, followed by normalization to an intraplate control. In Tables 1 and 1A, a designation of A indicates an IC₅₀ value of less than 1 µM, B indicates an IC₅₀ value of from 1 µM to less than 1 mM, and C indicates an IC₅₀ value of 1 mM or greater. One embodiment is a compound in Table 1, or a pharmaceutically acceptable salt thereof. One embodiment is a compound in Table 1A, or a pharmaceutically acceptable salt thereof.

TABLE 1

| | | |
|---|---|---|
| Compound No. | Compound Structure | Normalized in vitro IC₅₀ |
| KG-0336-0 | 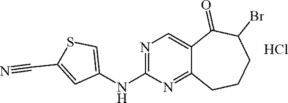 [SMILES: N#Cc1cc(Nc2ncc3c(n2)CCCC(Br)C3=O)cs1 ; Cl] | A |
| KG-0206-0 | 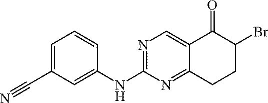 [SMILES: N#Cc1cccc(Nc2ncc3c(n2)CCC(Br)C3=O)c1] | A |
| KG-0316-0A | 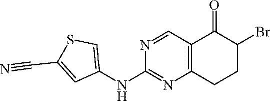 [SMILES: N#Cc1cc(Nc2ncc3c(n2)CCC(Br)C3=O)cs1] | A |
| KG-0338 | 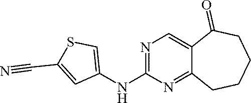 [SMILES: N#Cc1cc(Nc2ncc3c(n2)CCCCC3=O)cs1] | A |
| KG-0184-0A | 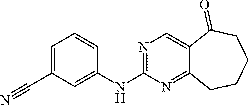 [SMILES: N#Cc1cccc(Nc2ncc3c(n2)CCCCC3=O)c1] | A |
| KG-0254-0A | 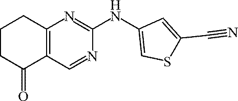 [SMILES: N#Cc1cc(Nc2ncc3c(n2)CCCC3=O)cs1] | A |
| KG-0205 | 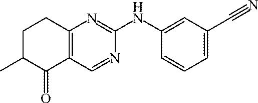 [SMILES: CC1CCc2nc(Nc3cccc(C#N)c3)ncc2C1=O] | A |
| KG-0318 | 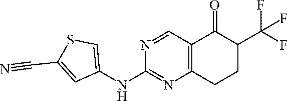 [SMILES: N#Cc1cc(Nc2ncc3c(n2)CCC(C(F)(F)F)C3=O)cs1] | A |
| KG-0317 | 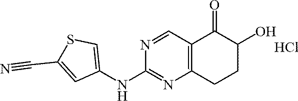 [SMILES: N#Cc1cc(Nc2ncc3c(n2)CCC(O)C3=O)cs1 ; Cl] | A |
| KG-0333-0 | 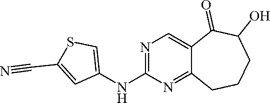 [SMILES: N#Cc1cc(Nc2ncc3c(n2)CCCC(O)C3=O)cs1] | A |
| KG-0315-0A | 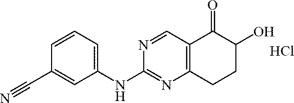 [SMILES: N#Cc1cccc(Nc2ncc3c(n2)CCC(O)C3=O)c1 ; Cl] | A |
| KG-0265-0 | 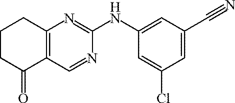 [SMILES: N#Cc1cc(Cl)cc(Nc2ncc3c(n2)CCCC3=O)c1] | A |
| KG-0206-0 | 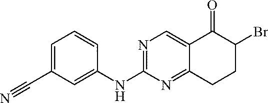 [SMILES: N#Cc1cccc(Nc2ncc3c(n2)CCC(Br)C3=O)c1] | A |
| KG-0316 | 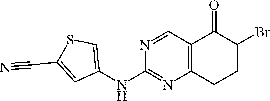 [SMILES: N#Cc1cc(Nc2ncc3c(n2)CCC(Br)C3=O)cs1] | A |
| KG-0334 | 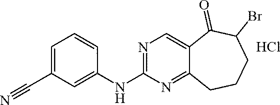 [SMILES: N#Cc1cccc(Nc2ncc3c(n2)CCCC(Br)C3=O)c1 ; Cl] | A |
| KG-0062-0 | 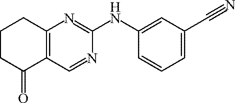 [SMILES: N#Cc1cccc(Nc2ncc3c(n2)CCCC3=O)c1] | A |
| KG-0065-0 | 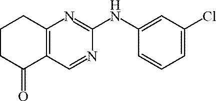 [SMILES: O=C1CCCc2nc(Nc3cccc(Cl)c3)ncc21] | A |
| KG-0255-0 | 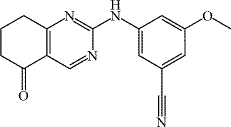 [SMILES: COc1cc(C#N)cc(Nc2ncc3c(n2)CCCC3=O)c1] | A |
| KG-0333 | 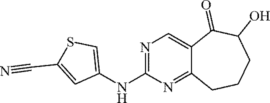 [SMILES: N#Cc1cc(Nc2ncc3c(n2)CCCC(O)C3=O)cs1] | A |
| KG-0167-0 | 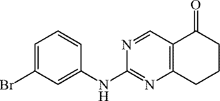 [SMILES: O=C1CCCc2nc(Nc3cccc(Br)c3)ncc21] | A |
| KG-0337-0 | 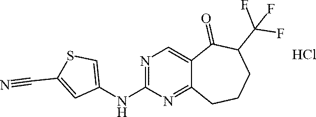 [SMILES: N#Cc1cc(Nc2ncc3c(n2)CCCC(C(F)(F)F)C3=O)cs1 ; Cl] | A |
| KG-0205-0 | 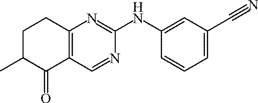 [SMILES: CC1CCc2nc(Nc3cccc(C#N)c3)ncc2C1=O] | A |
| KG-0335-0 | 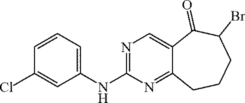 [SMILES: O=C1c2cnc(Nc3cccc(Cl)c3)nc2CCCC1Br] | A |
| KG-0321 | 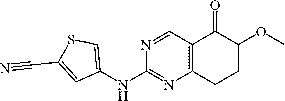 [SMILES: COC1CCc2nc(Nc3csc(C#N)c3)ncc2C1=O] | A |
| KG-0061-0 | 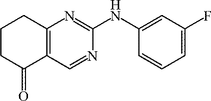 [SMILES: O=C1CCCc2nc(Nc3cccc(F)c3)ncc21] | A |
| KG-0232-0 | 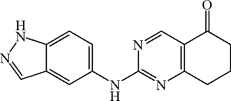 [SMILES: O=C1CCCc2nc(Nc3ccc4[nH]ncc4c3)ncc21] | A |
| KG-0256-0 | 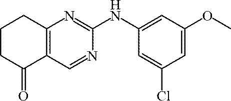 [SMILES: COc1cc(Cl)cc(Nc2ncc3c(n2)CCCC3=O)c1] | A |
| KG-0339 | 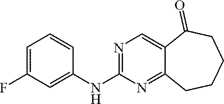 [SMILES: O=C1CCCCc2nc(Nc3cccc(F)c3)ncc21] | A |
| KG-0186-0A | 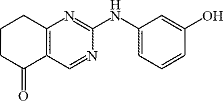 [SMILES: O=C1CCCc2nc(Nc3cccc(O)c3)ncc21] | A |
| KG-0229-0 | 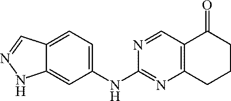 [SMILES: O=C1CCCc2nc(Nc3ccc4cn[nH]c4c3)ncc21] | B |
| KG-0187-0 | 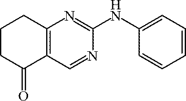 [SMILES: O=C1CCCc2nc(Nc3ccccc3)ncc21] | B |
| KG-0167 | 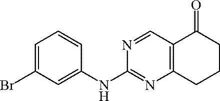 [SMILES: O=C1CCCc2nc(Nc3cccc(Br)c3)ncc21] | B |
| KG-0332-0 | 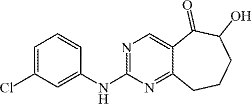 [SMILES: O=C1c2cnc(Nc3cccc(Cl)c3)nc2CCCC1O] | B |
| KG-0168-0 | 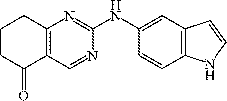 [SMILES: O=C1CCCc2nc(Nc3ccc4[nH]ccc4c3)ncc21] | B |
| KG-0166-0 | 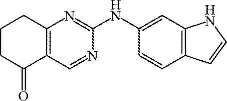 [SMILES: O=C1CCCc2nc(Nc3ccc4cc[nH]c4c3)ncc21] | B |
| KG-0252-0 | 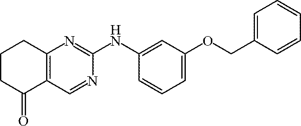 [SMILES: O=C1CCCc2nc(Nc3cccc(OCc4ccccc4)c3)ncc21] | B |
| KG-0183-0 | 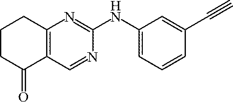 [SMILES: C#Cc1cccc(Nc2ncc3c(n2)CCCC3=O)c1] | B |
| KG-0190-0 | 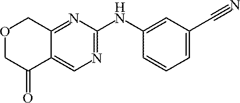 [SMILES: N#Cc1cccc(Nc2ncc3c(n2)COCC3=O)c1] | B |
| KG-0167-0A | 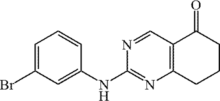 [SMILES: O=C1CCCc2nc(Nc3cccc(Br)c3)ncc21] | B |
| KG-0185-0 | 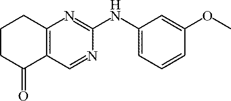 [SMILES: COc1cccc(Nc2ncc3c(n2)CCCC3=O)c1] | B |
| KG-0331 | 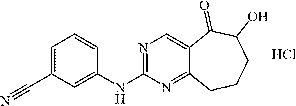 [SMILES: N#Cc1cccc(Nc2ncc3c(n2)CCCC(O)C3=O)c1 ; Cl] | B |
| KG-0239-0 | 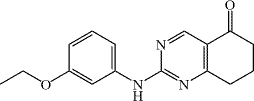 [SMILES: CCOc1cccc(Nc2ncc3c(n2)CCCC3=O)c1] | B |
| KG-0051-0 | 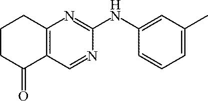 [SMILES: Cc1cccc(Nc2ncc3c(n2)CCCC3=O)c1] | B |
| KG-0283-0 | 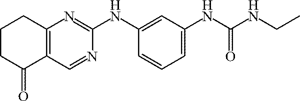 [SMILES: CCNC(=O)Nc1cccc(Nc2ncc3c(n2)CCCC3=O)c1] | B |
| KG-0228-0 | 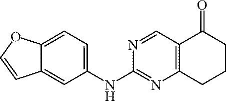 [SMILES: O=C1CCCc2nc(Nc3ccc4occc4c3)ncc21] | B |
| KG-0224-0 | 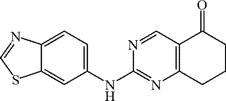 [SMILES: O=C1CCCc2nc(Nc3ccc4ncsc4c3)ncc21] | B |
| KG-0261-10 | 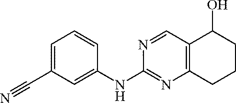 [SMILES: N#Cc1cccc(Nc2ncc3c(n2)CCCC3O)c1] | B |
| KG-0332 | 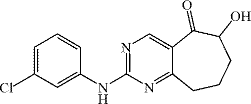 [SMILES: O=C1c2cnc(Nc3cccc(Cl)c3)nc2CCCC1O] | B |
| KG-0203-0 | 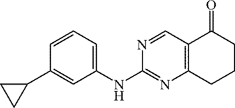 [SMILES: O=C1CCCc2nc(Nc3cccc(C4CC4)c3)ncc21] | B |
| KG-0204-0 | 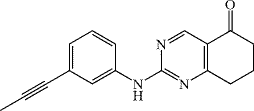 [SMILES: CC#Cc1cccc(Nc2ncc3c(n2)CCCC3=O)c1] | B |
| KG-0223-0 | 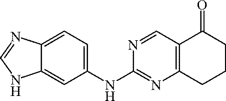 [SMILES: O=C1CCCc2nc(Nc3ccc4nc[nH]c4c3)ncc21] | B |
| KG-0252-0 | 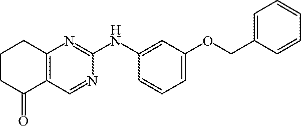 [SMILES: O=C1CCCc2nc(Nc3cccc(OCc4ccccc4)c3)ncc21] | B |
| KG-0252-0 | 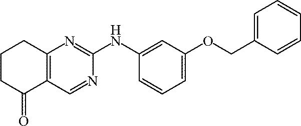 [SMILES: O=C1CCCc2nc(Nc3cccc(OCc4ccccc4)c3)ncc21] | B |
| KG-0227-0 | 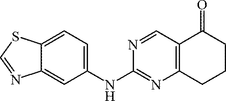 [SMILES: O=C1CCCc2nc(Nc3ccc4scnc4c3)ncc21] | B |
| KG-0251-0 | 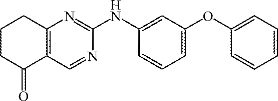 [SMILES: O=C1CCCc2nc(Nc3cccc(Oc4ccccc4)c3)ncc21] | B |
| KG-0249-0 | 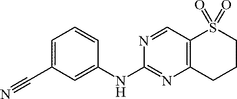 [SMILES: N#Cc1cccc(Nc2ncc3c(n2)CCCS3(=O)=O)c1] | B |
| KG-0064-0 | 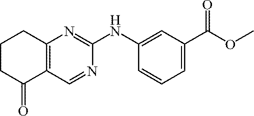 [SMILES: COC(=O)c1cccc(Nc2ncc3c(n2)CCCC3=O)c1] | B |
| KG-0337-0A | 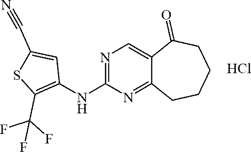 [SMILES: N#Cc1cc(Nc2ncc3c(n2)CCCCC3=O)c(C(F)(F)F)s1 ; Cl] | B |
| KG-0198-0 | 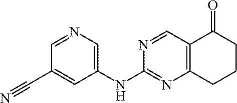 [SMILES: N#Cc1cncc(Nc2ncc3c(n2)CCCC3=O)c1] | B |
| KG-0240-0 | 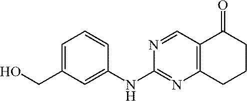 [SMILES: O=C1CCCc2nc(Nc3cccc(CO)c3)ncc21] | B |
| KG-0242-0 | 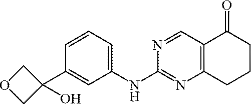 [SMILES: O=C1CCCc2nc(Nc3cccc(C4(O)COC4)c3)ncc21] | B |
| KG-0241-0 | 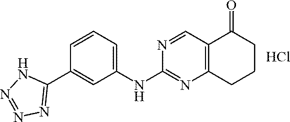 [SMILES: O=C1CCCc2nc(Nc3cccc(-c4nnn[nH]4)c3)ncc21 ; Cl] | B |
| KG-0164-0 | 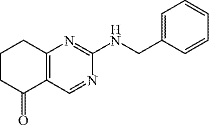 [SMILES: O=C1CCCc2nc(NCc3ccccc3)ncc21] | B |
| KG-0188-0 | 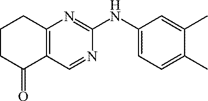 [SMILES: Cc1ccc(Nc2ncc3c(n2)CCCC3=O)cc1C] | B |
| KG-0238-0 | 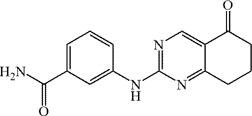 [SMILES: NC(=O)c1cccc(Nc2ncc3c(n2)CCCC3=O)c1] | B |
| KG-0055-0 | 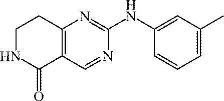 [SMILES: Cc1cccc(Nc2ncc3c(n2)CCNC3=O)c1] | B |
| KG-0056-0A | 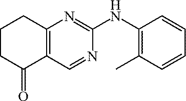 [SMILES: Cc1ccccc1Nc1ncc2c(n1)CCCC2=O] | B |
| KG-0138-0 | 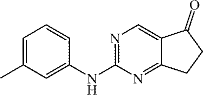 [SMILES: Cc1cccc(Nc2ncc3c(n2)CCC3=O)c1] | B |
| KG-0165-0 | 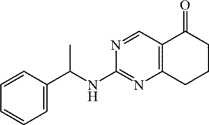 [SMILES: CC(Nc1ncc2c(n1)CCCC2=O)c1ccccc1] | B |
| KG-0215-0 | 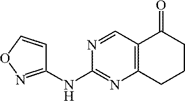 [SMILES: O=C1CCCc2nc(Nc3ccon3)ncc21] | B |
| KG-0219-0 | 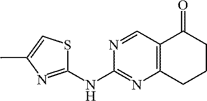 [SMILES: Cc1csc(Nc2ncc3c(n2)CCCC3=O)n1] | B |
| KG-0163-0 | 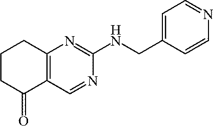 [SMILES: O=C1CCCc2nc(NCc3ccncc3)ncc21] | B |
| KG-0209-0 | 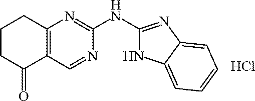 [SMILES: O=C1CCCc2nc(Nc3nc4ccccc4[nH]3)ncc21 ; Cl] | B |
| KG-0162-0 | 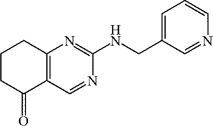 [SMILES: O=C1CCCc2nc(NCc3cccnc3)ncc21] | B |
| KG-0063-0 | 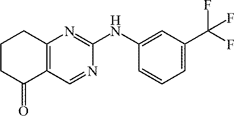 [SMILES: O=C1CCCc2nc(Nc3cccc(C(F)(F)F)c3)ncc21] | B |
| KG-0214-0 | 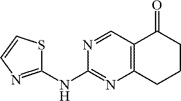 [SMILES: O=C1CCCc2nc(Nc3nccs3)ncc21] | B |
| KG-0220-0 | 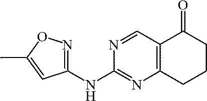 [SMILES: Cc1cc(Nc2ncc3c(n2)CCCC3=O)no1] | B |
| KG-0216-0 | 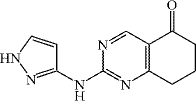 [SMILES: O=C1CCCc2nc(Nc3cc[nH]n3)ncc21] | C |
| KG-0237-0 | 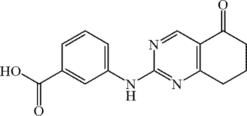 [SMILES: O=C(O)c1cccc(Nc2ncc3c(n2)CCCC3=O)c1] | C |
| KG-0060-0 | 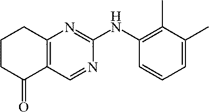 [SMILES: Cc1cccc(Nc2ncc3c(n2)CCCC3=O)c1C] | C |
| KG-0176 | 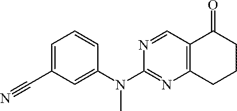 [SMILES: CN(c1cccc(C#N)c1)c1ncc2c(n1)CCCC2=O] | C |
| KG-0141-0 | 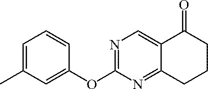 [SMILES: Cc1cccc(Oc2ncc3c(n2)CCCC3=O)c1] | C |
| KG-0165-0 | 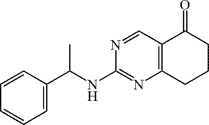 [SMILES: CC(Nc1ncc2c(n1)CCCC2=O)c1ccccc1] | C |
| KG-0167-0 | 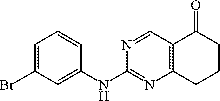 [SMILES: O=C1CCCc2nc(Nc3cccc(Br)c3)ncc21] | C |
| KG-0211-0 | 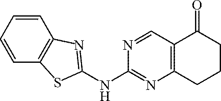 [SMILES: O=C1CCCc2nc(Nc3nc4ccccc4s3)ncc21] | C |
| KG-0213-0 | 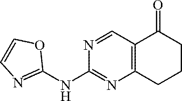 [SMILES: O=C1CCCc2nc(Nc3ncco3)ncc21] | C |
| KG-0258-0 | 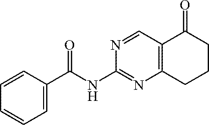 [SMILES: O=C(Nc1ncc2c(n1)CCCC2=O)c1ccccc1] | C |
| KG-0260-0 | 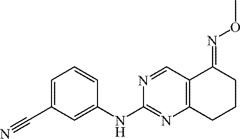 [SMILES: CO/N=C1\CCCc2nc(Nc3cccc(C#N)c3)ncc21] | C |

TABLE 1A

| | | |
|---|---|---|
| Compound No. | Compound Structure | Normalized in vitro IC₅₀ |
| 0001 | 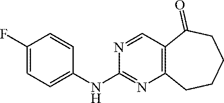 [SMILES: O=C1CCCCc2nc(Nc3ccc(F)cc3)ncc21] | B |
| 0002 | 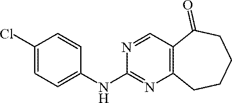 [SMILES: O=C1CCCCc2nc(Nc3ccc(Cl)cc3)ncc21] | B |
| 0003 | 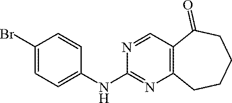 [SMILES: O=C1CCCCc2nc(Nc3ccc(Br)cc3)ncc21] | B |
| 0004 | 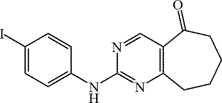 [SMILES: O=C1CCCCc2nc(Nc3ccc(I)cc3)ncc21] | B |
| 0005 | 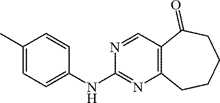 [SMILES: Cc1ccc(Nc2ncc3c(n2)CCCCC3=O)cc1] | B |
| 0006 | 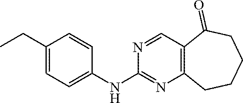 [SMILES: CCc1ccc(Nc2ncc3c(n2)CCCCC3=O)cc1] | B |
| 0007 | 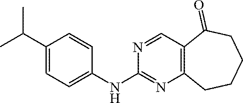 [SMILES: CC(C)c1ccc(Nc2ncc3c(n2)CCCCC3=O)cc1] | B |
| 0008 | 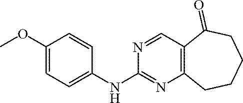 [SMILES: COc1ccc(Nc2ncc3c(n2)CCCCC3=O)cc1] | B |
| 0009 | 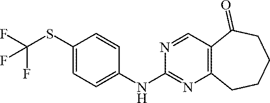 [SMILES: O=C1CCCCc2nc(Nc3ccc(SC(F)(F)F)cc3)ncc21] | B |
| 0010 | 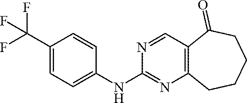 [SMILES: O=C1CCCCc2nc(Nc3ccc(C(F)(F)F)cc3)ncc21] | B |
| 0011 | 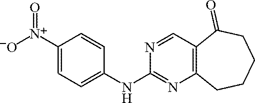 [SMILES: O=C1CCCCc2nc(Nc3ccc([N+](=O)[O-])cc3)ncc21] | B |
| 0012 | 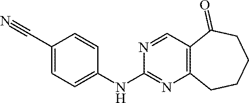 [SMILES: N#Cc1ccc(Nc2ncc3c(n2)CCCCC3=O)cc1] | B |
| 0013 | 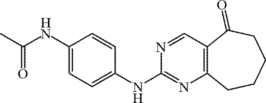 [SMILES: CC(=O)Nc1ccc(Nc2ncc3c(n2)CCCCC3=O)cc1] | C |
| 0014 | 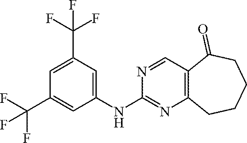 [SMILES: O=C1CCCCc2nc(Nc3cc(C(F)(F)F)cc(C(F)(F)F)c3)ncc21] | B |
| 0015 | 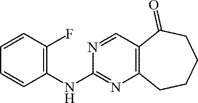 [SMILES: O=C1CCCCc2nc(Nc3ccccc3F)ncc21] | B |
| 0016 | 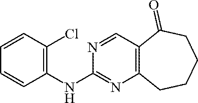 [SMILES: O=C1CCCCc2nc(Nc3ccccc3Cl)ncc21] | B |
| 0017 | 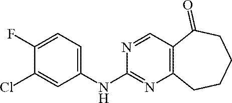 [SMILES: O=C1CCCCc2nc(Nc3ccc(F)c(Cl)c3)ncc21] | A |
| 0018 | 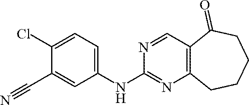 [SMILES: N#Cc1cc(Nc2ncc3c(n2)CCCCC3=O)ccc1Cl] | B |
| 0019 | 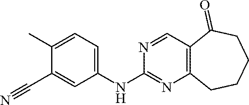 [SMILES: Cc1ccc(Nc2ncc3c(n2)CCCCC3=O)cc1C#N] | A |
| 0020 | 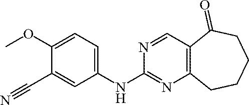 [SMILES: COc1ccc(Nc2ncc3c(n2)CCCCC3=O)cc1C#N] | A |
| 0021 | 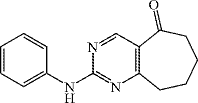 [SMILES: O=C1CCCCc2nc(Nc3ccccc3)ncc21] | B |
| 0022 | 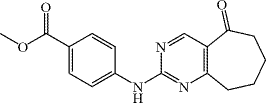 [SMILES: COC(=O)c1ccc(Nc2ncc3c(n2)CCCCC3=O)cc1] | B |
| 0023 | 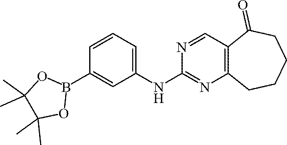 [SMILES: CC1(C)OB(c2cccc(Nc3ncc4c(n3)CCCCC4=O)c2)OC1(C)C] | B |
| 0024 | 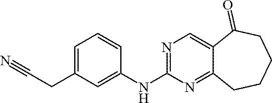 [SMILES: N#CCc1cccc(Nc2ncc3c(n2)CCCCC3=O)c1] | B |
| 0025 | 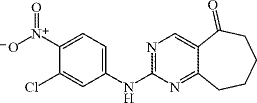 [SMILES: O=C1CCCCc2nc(Nc3ccc([N+](=O)[O-])c(Cl)c3)ncc21] | B |
| 0026 | 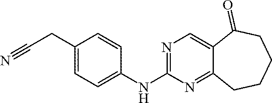 [SMILES: N#CCc1ccc(Nc2ncc3c(n2)CCCCC3=O)cc1] | B |
| 0027 | 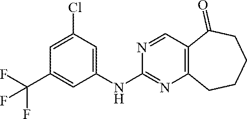 [SMILES: O=C1CCCCc2nc(Nc3cc(Cl)cc(C(F)(F)F)c3)ncc21] | B |
| 0028 | 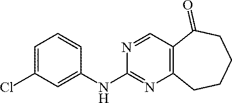 [SMILES: O=C1CCCCc2nc(Nc3cccc(Cl)c3)ncc21] | A |
| 0031 | 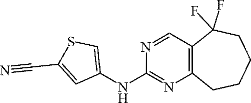 [SMILES: N#Cc1cc(Nc2ncc3c(n2)CCCCC3(F)F)cs1] | B |
| 0032 | 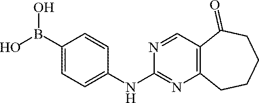 [SMILES: O=C1CCCCc2nc(Nc3ccc(B(O)O)cc3)ncc21] | B |
| 0033 | 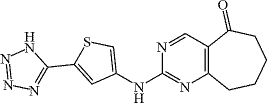 [SMILES: O=C1CCCCc2nc(Nc3csc(-c4nnn[nH]4)c3)ncc21] | B |
| 0034 | 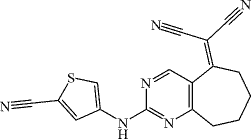 [SMILES: N#CC(C#N)=C1CCCCc2nc(Nc3csc(C#N)c3)ncc21] | A |
| 0035 | 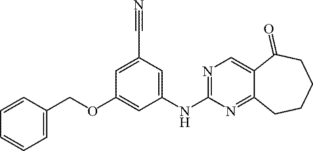 [SMILES: N#Cc1cc(Nc2ncc3c(n2)CCCCC3=O)cc(OCc2ccccc2)c1] | A |
| 0036 | 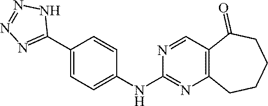 [SMILES: O=C1CCCCc2nc(Nc3ccc(-c4nnn[nH]4)cc3)ncc21] | A |
| 0040 | 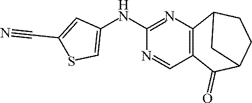 [SMILES: N#Cc1cc(Nc2ncc3c(n2)C2CCC(C2)C3=O)cs1] | B |
| 0042 | 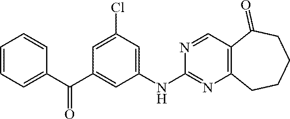 [SMILES: O=C(c1ccccc1)c1cc(Cl)cc(Nc2ncc3c(n2)CCCCC3=O)c1] | B |
| 0043 | 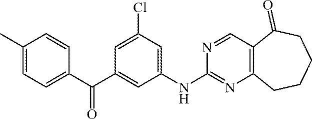 [SMILES: Cc1ccc(C(=O)c2cc(Cl)cc(Nc3ncc4c(n3)CCCCC4=O)c2)cc1] | C |
| 0044 | 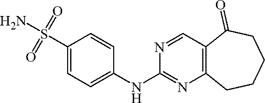 [SMILES: NS(=O)(=O)c1ccc(Nc2ncc3c(n2)CCCCC3=O)cc1] | B |
| 0045 | 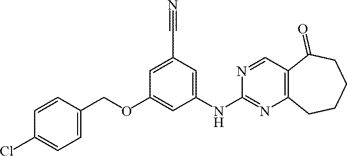 [SMILES: N#Cc1cc(Nc2ncc3c(n2)CCCCC3=O)cc(OCc2ccc(Cl)cc2)c1] | B |
| 0046 | 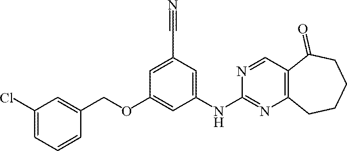 [SMILES: N#Cc1cc(Nc2ncc3c(n2)CCCCC3=O)cc(OCc2cccc(Cl)c2)c1] | A |
| 0047 | 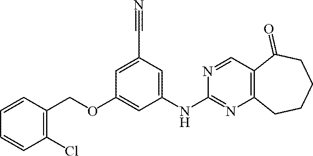 [SMILES: N#Cc1cc(Nc2ncc3c(n2)CCCCC3=O)cc(OCc2ccccc2Cl)c1] | A |
| 0048 | 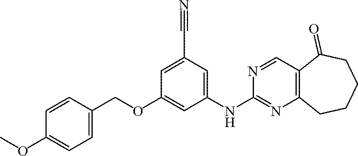 [SMILES: COc1ccc(COc2cc(C#N)cc(Nc3ncc4c(n3)CCCCC4=O)c2)cc1] | A |
| 0049 | 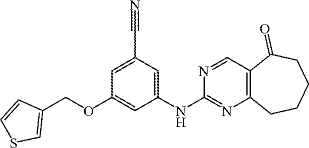 [SMILES: N#Cc1cc(Nc2ncc3c(n2)CCCCC3=O)cc(OCc2ccsc2)c1] | A |
| 0050 | 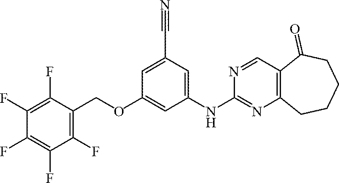 [SMILES: N#Cc1cc(Nc2ncc3c(n2)CCCCC3=O)cc(OCc2c(F)c(F)c(F)c(F)c2F)c1] | A |
| 0051 | 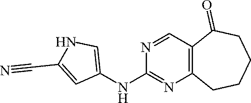 [SMILES: N#Cc1cc(Nc2ncc3c(n2)CCCCC3=O)c[nH]1] | A |
| 0052 | 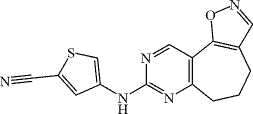 [SMILES: N#Cc1cc(Nc2ncc3c(n2)CCCc2cnoc2-3)cs1] | B |
| 0053 | 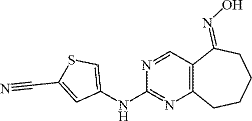 [SMILES: N#Cc1cc(Nc2ncc3c(n2)CCCC/C3=N\O)cs1] | C |
| 0054 | 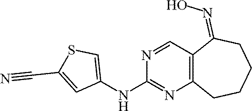 [SMILES: N#Cc1cc(Nc2ncc3c(n2)CCCC/C3=N/O)cs1] | C |
| 0055 | 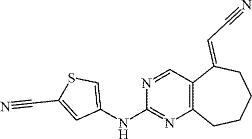 [SMILES: N#C/C=C1\CCCCc2nc(Nc3csc(C#N)c3)ncc21] | B |
| 0056 | 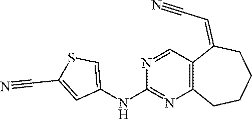 [SMILES: N#C/C=C1/CCCCc2nc(Nc3csc(C#N)c3)ncc21] | B |
| 0057 | 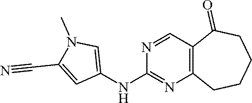 [SMILES: Cn1cc(Nc2ncc3c(n2)CCCCC3=O)cc1C#N] | B |
| 0058 | 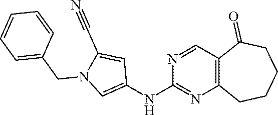 [SMILES: N#Cc1cc(Nc2ncc3c(n2)CCCCC3=O)cn1Cc1ccccc1] | B |
| 0059 | 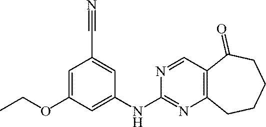 [SMILES: CCOc1cc(C#N)cc(Nc2ncc3c(n2)CCCCC3=O)c1] | A |
| 0060 | 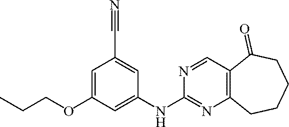 [SMILES: CCCOc1cc(C#N)cc(Nc2ncc3c(n2)CCCCC3=O)c1] | A |
| 0061 | 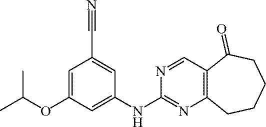 [SMILES: CC(C)Oc1cc(C#N)cc(Nc2ncc3c(n2)CCCCC3=O)c1] | A |
| 0062 | 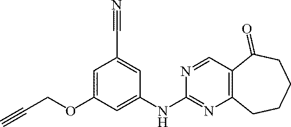 [SMILES: C#CCOc1cc(C#N)cc(Nc2ncc3c(n2)CCCCC3=O)c1] | A |
| 0063 | 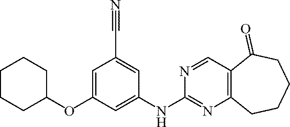 [SMILES: N#Cc1cc(Nc2ncc3c(n2)CCCCC3=O)cc(OC2CCCCC2)c1] | A |
| 0065 | 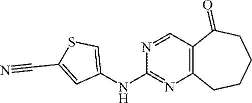 [SMILES: N#Cc1cc(Nc2ncc3c(n2)CCCCC3=O)cs1] | A |
| 0066 | 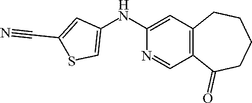 [SMILES: N#Cc1cc(Nc2cc3c(cn2)C(=O)CCCC3)cs1] | B |
| 0067 | 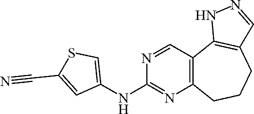 [SMILES: N#Cc1cc(Nc2ncc3c(n2)CCCc2cn[nH]c2-3)cs1] | B |
| 0071 | 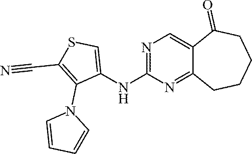 [SMILES: N#Cc1scc(Nc2ncc3c(n2)CCCCC3=O)c1-n1cccc1] | A |
| 0072 | 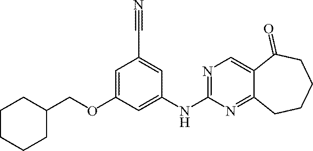 [SMILES: N#Cc1cc(Nc2ncc3c(n2)CCCCC3=O)cc(OCC2CCCCC2)c1] | B |
| 0073 | 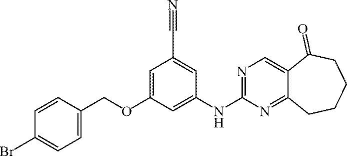 [SMILES: N#Cc1cc(Nc2ncc3c(n2)CCCCC3=O)cc(OCc2ccc(Br)cc2)c1] | B |
| 0074 | 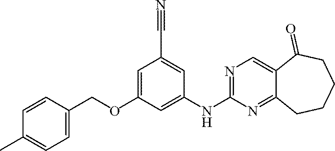 [SMILES: Cc1ccc(COc2cc(C#N)cc(Nc3ncc4c(n3)CCCCC4=O)c2)cc1] | A |
| 0075 | 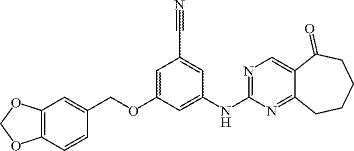 [SMILES: N#Cc1cc(Nc2ncc3c(n2)CCCCC3=O)cc(OCc2ccc3c(c2)OCO3)c1] | A |
| 0076 | 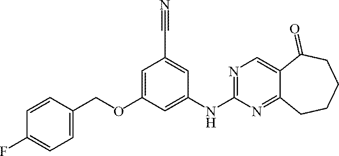 [SMILES: N#Cc1cc(Nc2ncc3c(n2)CCCCC3=O)cc(OCc2ccc(F)cc2)c1] | A |
| 0077 | 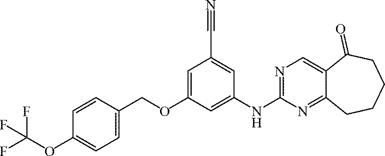 [SMILES: N#Cc1cc(Nc2ncc3c(n2)CCCCC3=O)cc(OCc2ccc(OC(F)(F)F)cc2)c1] | B |
| 0078 | 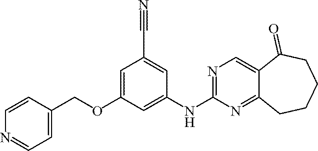 [SMILES: N#Cc1cc(Nc2ncc3c(n2)CCCCC3=O)cc(OCc2ccncc2)c1] | A |
| 0079 | 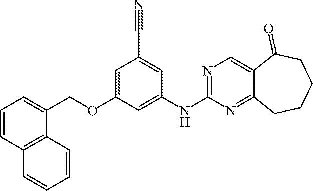 [SMILES: N#Cc1cc(Nc2ncc3c(n2)CCCCC3=O)cc(OCc2cccc3ccccc23)c1] | A |
| 0080 | 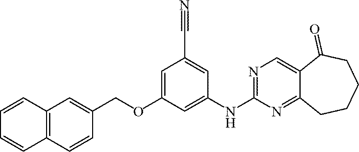 [SMILES: N#Cc1cc(Nc2ncc3c(n2)CCCCC3=O)cc(OCc2ccc3ccccc3c2)c1] | B |
| 0081 | 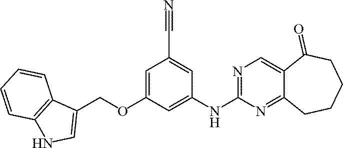 [SMILES: N#Cc1cc(Nc2ncc3c(n2)CCCCC3=O)cc(OCc2c[nH]c3ccccc23)c1] | C |
| 0082 | 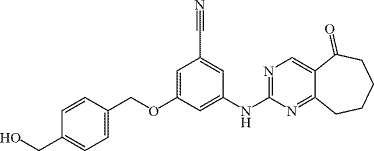 [SMILES: N#Cc1cc(Nc2ncc3c(n2)CCCCC3=O)cc(OCc2ccc(CO)cc2)c1] | A |
| 0083 | 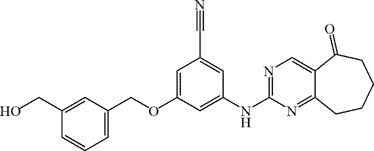 [SMILES: N#Cc1cc(Nc2ncc3c(n2)CCCCC3=O)cc(OCc2cccc(CO)c2)c1] | A |
| 0084 | 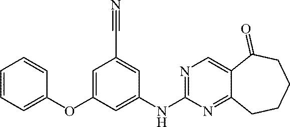 [SMILES: N#Cc1cc(Nc2ncc3c(n2)CCCCC3=O)cc(Oc2ccccc2)c1] | B |
| 0085 | 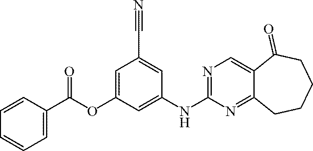 [SMILES: N#Cc1cc(Nc2ncc3c(n2)CCCCC3=O)cc(OC(=O)c2ccccc2)c1] | B |
| 0086 | 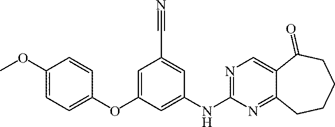 [SMILES: COc1ccc(Oc2cc(C#N)cc(Nc3ncc4c(n3)CCCCC4=O)c2)cc1] | B |
| 0087 | 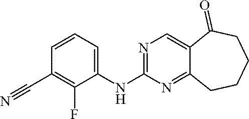 [SMILES: N#Cc1cccc(Nc2ncc3c(n2)CCCCC3=O)c1F] | A |
| 0088 | 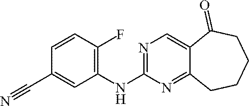 [SMILES: N#Cc1ccc(F)c(Nc2ncc3c(n2)CCCCC3=O)c1] | B |
| 0089 | 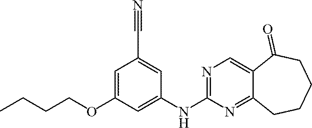 [SMILES: CCCCOc1cc(C#N)cc(Nc2ncc3c(n2)CCCCC3=O)c1] | B |
| 0090 | 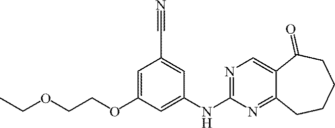 [SMILES: CCOCCOc1cc(C#N)cc(Nc2ncc3c(n2)CCCCC3=O)c1] | B |
| 0092 | 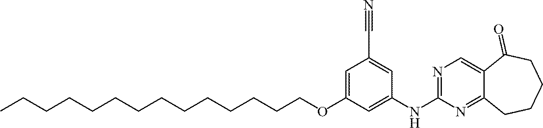 [SMILES: CCCCCCCCCCCCCCOc1cc(C#N)cc(Nc2ncc3c(n2)CCCCC3=O)c1] | C |
| 0093 | 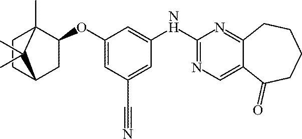 [SMILES: CC1(C)[C@H]2CCC1(C)[C@@H](Oc1cc(C#N)cc(Nc3ncc4c(n3)CCCCC4=O)c1)C2] | C |
| 0094 | 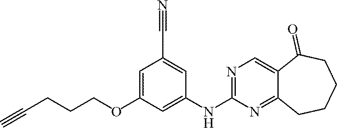 [SMILES: C#CCCCOc1cc(C#N)cc(Nc2ncc3c(n2)CCCCC3=O)c1] | A |
| 0095 | 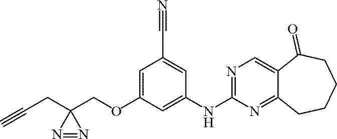 [SMILES: C#CCC1(COc2cc(C#N)cc(Nc3ncc4c(n3)CCCCC4=O)c2)N=N1] | C |
| 0096 | 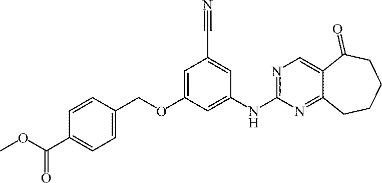 [SMILES: COC(=O)c1ccc(COc2cc(C#N)cc(Nc3ncc4c(n3)CCCCC4=O)c2)cc1] | C |
| 0097 | 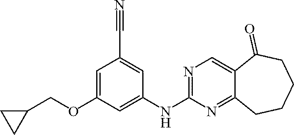 [SMILES: N#Cc1cc(Nc2ncc3c(n2)CCCCC3=O)cc(OCC2CC2)c1] | A |
| 0098 | 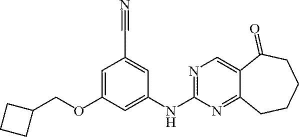 [SMILES: N#Cc1cc(Nc2ncc3c(n2)CCCCC3=O)cc(OCC2CCC2)c1] | B |
| 0099 | 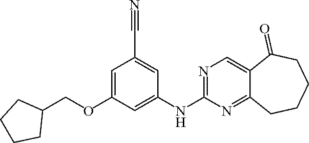 [SMILES: N#Cc1cc(Nc2ncc3c(n2)CCCCC3=O)cc(OCC2CCCC2)c1] | B |
| 0100 | 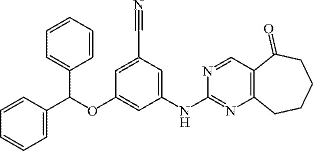 [SMILES: N#Cc1cc(Nc2ncc3c(n2)CCCCC3=O)cc(OC(c2ccccc2)c2ccccc2)c1] | B |
| 0101 | 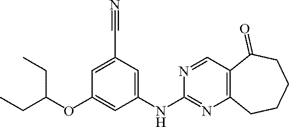 [SMILES: CCC(CC)Oc1cc(C#N)cc(Nc2ncc3c(n2)CCCCC3=O)c1] | B |
| 0102 | 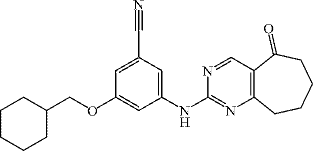 [SMILES: N#Cc1cc(Nc2ncc3c(n2)CCCCC3=O)cc(OCC2CCCCC2)c1] | B |
| 0103 | 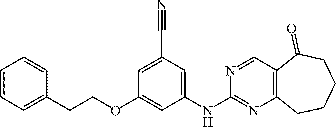 [SMILES: N#Cc1cc(Nc2ncc3c(n2)CCCCC3=O)cc(OCCc2ccccc2)c1] | A |
| 0104 | 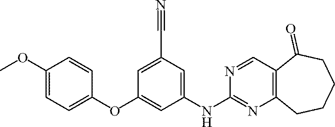 [SMILES: COc1ccc(Oc2cc(C#N)cc(Nc3ncc4c(n3)CCCCC4=O)c2)cc1] | B |
| 0105 | 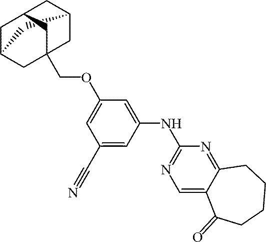 [SMILES: N#Cc1cc(Nc2ncc3c(n2)CCCCC3=O)cc(OCC23C[C@H]4C[C@@H](C2)C[C@@H](C3)C4)c1] | C |
| 0106 | 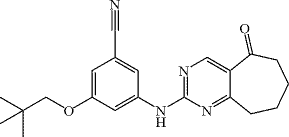 [SMILES: CC(C)(C)COc1cc(C#N)cc(Nc2ncc3c(n2)CCCCC3=O)c1] | C |
| 0121 | 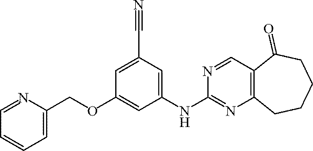 [SMILES: N#Cc1cc(Nc2ncc3c(n2)CCCCC3=O)cc(OCc2ccccn2)c1] | A |
| 0122 | 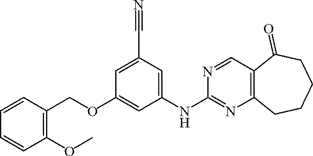 [SMILES: COc1ccccc1COc1cc(C#N)cc(Nc2ncc3c(n2)CCCCC3=O)c1] | A |
| 0123 | 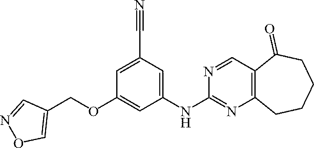 [SMILES: N#Cc1cc(Nc2ncc3c(n2)CCCCC3=O)cc(OCc2cnoc2)c1] | A |
| 0124 | 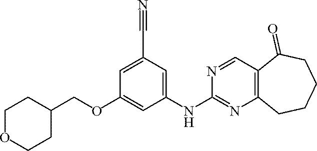 [SMILES: N#Cc1cc(Nc2ncc3c(n2)CCCCC3=O)cc(OCC2CCOCC2)c1] | A |
| 0135 | 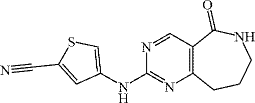 [SMILES: N#Cc1cc(Nc2ncc3c(n2)CCCNC3=O)cs1] | B |
| 0136 | 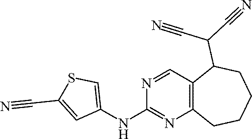 [SMILES: N#Cc1cc(Nc2ncc3c(n2)CCCCC3C(C#N)C#N)cs1] | B |
| 0118 | 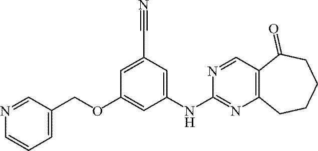 [SMILES: N#Cc1cc(Nc2ncc3c(n2)CCCCC3=O)cc(OCc2cccnc2)c1] | A |
| 0125 | 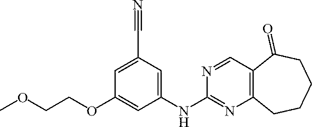 [SMILES: COCCOc1cc(C#N)cc(Nc2ncc3c(n2)CCCCC3=O)c1] | A |
| 0128 | 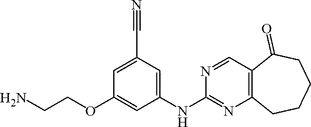 [SMILES: N#Cc1cc(Nc2ncc3c(n2)CCCCC3=O)cc(OCCN)c1] | B |
| 0161 | 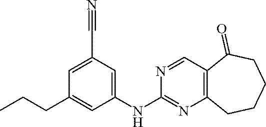 [SMILES: CCCc1cc(C#N)cc(Nc2ncc3c(n2)CCCCC3=O)c1] | B |
| 0162 | 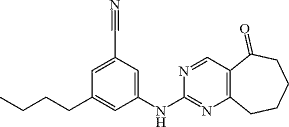 [SMILES: CCCCc1cc(C#N)cc(Nc2ncc3c(n2)CCCCC3=O)c1] | B |
| 0163 | 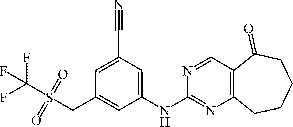 [SMILES: N#Cc1cc(CS(=O)(=O)C(F)(F)F)cc(Nc2ncc3c(n2)CCCCC3=O)c1] | B |
| KG-0052 | 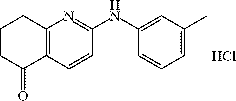 [SMILES: Cc1cccc(Nc2ccc3c(n2)CCCC3=O)c1 ; Cl] | C |
| KG-0054 | 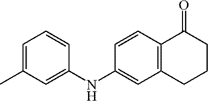 [SMILES: Cc1cccc(Nc2ccc3c(c2)CCCC3=O)c1] | C |
| KG-0057 | 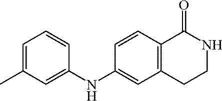 [SMILES: Cc1cccc(Nc2ccc3c(c2)CCNC3=O)c1] | C |

### Compositions

Also provided herein is a pharmaceutical composition, comprising a compound disclosed herein (e.g., a compound of any one of structural formulas I-VI), or a pharmaceutically acceptable salt thereof, and a pharmaceutically acceptable carrier. The compositions can be used in the methods described herein, e.g., to supply a compound described herein, or a pharmaceutically acceptable salt thereof.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions described herein may be administered orally, parenterally (including subcutaneously, intramuscularly, intravenously and intradermally), by inhalation spray, topically, rectally, nasally, buccally, vaginally or via an implanted reservoir. In some embodiments, provided compounds or compositions are administrable intravenously and/or intraperitoneally.

The term “parenteral,” as used herein, includes subcutaneous, intracutaneous, intravenous, intramuscular, intraocular, intravitreal, intra-articular, intra-arterial, intrasynovial, intrasternal, intrathecal, intralesional, intrahepatic, intraperitoneal intralesional and intracranial injection or infusion techniques. Preferably, the compositions are administered orally, subcutaneously, intraperitoneally or intravenously.

Compositions provided herein can be orally administered in any orally acceptable dosage form including, but not limited to, capsules, tablets, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

In some embodiments, an oral formulation is formulated for immediate release or sustained/delayed release.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the compound of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants such as wetting agents, emulsifying and suspending agents, sweetening, flavoring, coloring, perfuming, and preservative agents.

Compositions suitable for buccal or sublingual administration include tablets, lozenges and pastilles, wherein the active ingredient is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

A compound described herein can also be in micro-encapsulated form with one or more excipients, as noted above. In such solid dosage forms, the compound can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the active ingredient against degradation as it passes through the alimentary tract, for example, by an outer coating of the formulation on a tablet or capsule.

In another embodiment, a compound or pharmaceutically acceptable salt described herein can be provided in an extended (or “delayed” or “sustained”) release composition. This delayed-release composition comprises the compound or pharmaceutically acceptable salt in combination with a delayed-release component. Such a composition allows targeted release of a provided agent into the lower gastrointestinal tract, for example, into the small intestine, the large intestine, the colon and/or the rectum. In certain embodiments, a delayed-release composition further comprises an enteric or pH-dependent coating, such as cellulose acetate phthalates and other phthalates (e.g., polyvinyl acetate phthalate, methacrylates (Eudragits)). Alternatively, the delayed-release composition provides controlled release to the small intestine and/or colon by the provision of pH sensitive methacrylate coatings, pH sensitive polymeric microspheres, or polymers which undergo degradation by hydrolysis. The delayed-release composition can be formulated with hydrophobic or gelling excipients or coatings. Colonic delivery can further be provided by coatings which are digested by bacterial enzymes such as amylose or pectin, by pH dependent polymers, by hydrogel plugs swelling with time (Pulsincap), by time-dependent hydrogel coatings and/or by acrylic acid linked to azoaromatic bonds coatings.

Compositions described herein can also be administered subcutaneously, intraperitoneally or intravenously. Compositions described herein for intravenous, subcutaneous, or intraperitoneal injection may contain an isotonic vehicle such as sodium chloride injection, Ringer’s injection, dextrose injection, dextrose and sodium chloride injection, lactated Ringer’s injection, or other vehicles known in the art.

Compositions described herein can also be administered in the form of suppositories for rectal administration. These can be prepared by mixing a compound described herein with a suitable non-irritating excipient that is solid at room temperature but liquid at rectal temperature and, therefore, will melt in the rectum to release the drug. Such materials include cocoa butter, beeswax and polyethylene glycols.

Compositions described herein can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For other topical applications, the compositions can be formulated in a suitable ointment containing the active component suspended or dissolved in one or more carriers. Carriers for topical administration of a compound described herein include, but are not limited to, mineral oil, liquid petrolatum, white petrolatum, propylene glycol, polyoxyethylene, polyoxypropylene compound, emulsifying wax and water and penetration enhancers. Alternatively, compositions can be formulated in a suitable lotion or cream containing the active compound suspended or dissolved in one or more pharmaceutically acceptable carriers. Alternatively, the composition can be formulated with a suitable lotion or cream containing the active compound suspended or dissolved in a carrier with suitable emulsifying agents. In some embodiments, suitable carriers include, but are not limited to, mineral oil, sorbitan monostearate, polysorbate 60, cetyl esters wax, cetearyl alcohol, 2-octyldodecanol, benzyl alcohol and water. In other embodiments, suitable carriers include, but are not limited to, mineral oil, sorbitan monostearate, polysorbate 60, cetyl esters wax, cetearyl alcohol, 2-octyldodecanol, benzyl alcohol and water and penetration enhancers.

For ophthalmic use, compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic use, the compositions can be formulated in an ointment such as petrolatum.

Compositions can also be administered by nasal aerosol or inhalation, for example, for the treatment of asthma. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents. Without wishing to be bound by any particular theory, it is believed that local delivery of a composition described herein, as can be achieved by nasal aerosol or inhalation, for example, can reduce the risk of systemic consequences of the composition, for example, consequences for red blood cells.

The amount of a compound described herein, or a pharmaceutically acceptable salt thereof, that can be combined with the carrier materials to produce a composition in a single dosage form will vary depending upon the host treated, the particular mode of administration and the activity of the agent employed. Preferably, compositions should be formulated so that a dosage of from about 0.01 mg/kg to about 100 mg/kg body weight/day of the compound, or pharmaceutically acceptable salt thereof, can be administered to a subject receiving the composition.

The desired dose may conveniently be administered in a single dose or as multiple doses administered at appropriate intervals such that, for example, the agent is administered 2, 3, 4, 5, 6 or more times per day. The daily dose can be divided, especially when relatively large amounts are administered, or as deemed appropriate, into several, for example 2, 3, 4, 5, 6 or more, administrations.

It should also be understood that a specific dosage and treatment regimen for any particular patient will depend upon a variety of factors, including the activity of the specific agent employed, the age, body weight, general health, sex, diet, time of administration, rate of excretion, drug combination, the judgment of the treating physician and the severity of the particular disease being treated. The amount of a compound in the composition will also depend upon the particular compound in the composition.

Other pharmaceutically acceptable carriers, adjuvants and vehicles that can be used in the compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl- β-cyclodextrins, or other solubilized derivatives can also be advantageously used to enhance delivery of agents described herein.

The compositions can be in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer’s solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of inj ectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation.

In some embodiments, compositions comprising a compound described herein (e.g., a compound of any one of structural formulas I-VI), or a pharmaceutically acceptable salt thereof, can also include one or more other therapeutic agents, e.g., in combination. When the compositions of this invention comprise a combination, the agents should be present at dosage levels of between about 1 to 100%, and more preferably between about 5% to about 95% of the dosage normally administered in a monotherapy regimen.

Also provided herein is a kit comprising a compound described herein (e.g., a compound of any of structural formulas I-VI), or a pharmaceutically acceptable salt thereof, and an additional therapeutic agent(s). In one embodiment, the kit comprises an effective amount of a compound described herein, or a pharmaceutically acceptable salt thereof, to treat a disease, disorder or condition described herein, and an effective amount of an additional therapeutic agent(s) to treat the disease, disorder or condition. In some embodiments, the kit further comprises written instructions for administering the compound, or a pharmaceutically acceptable salt thereof, and the additional agent(s) to a subject to treat a disease, disorder or condition described herein.

The compositions described herein can, for example, be administered by injection, intravenously, intraarterially, intraocularly, intravitreally, subdermally, orally, buccally, nasally, transmucosally, topically, in an ophthalmic preparation, or by inhalation, with a dosage ranging from about 0.5 mg/kg to about 100 mg/kg of body weight or, alternatively, in a dosage ranging from about 1 mg/dose to about 1000 mg/dose, every 4 to 120 hours, or according to the requirements of the particular drug. Typically, the compositions will be administered from about 1 to about 6 (e.g., 1, 2, 3, 4, 5 or 6) times per day or, alternatively, as an infusion (e.g., a continuous infusion). The amount of active ingredient that can be combined with a carrier material to produce a single dosage form will vary depending upon the host treated and the particular mode of administration. A typical preparation will contain from about 1% to about 95%, from about 2.5% to about 95% or from about 5% to about 95% active compound (w/w). Alternatively, a preparation can contain from about 20% to about 80% active compound (w/w).

Doses lower or higher than those recited above may be required. Specific dosage and treatment regimens for any particular patient will depend upon a variety of factors, including the activity of the specific agent employed, the age, body weight, general health status, sex, diet, time of administration, rate of excretion, drug combination, the severity and course of the disease, condition or symptoms, the patient’s disposition to the disease, condition or symptoms, and the judgment of the treating physician.

### Methods

Mammalian cells require the reduced cofactor NADPH for the biosynthesis of lipids, nucleotides and amino acids, for producing “oxidative burst” and for preserving cellular redox balance. To generate cytosolic NADPH, cells use malic enzyme (ME1), isocitrate dehydrogenase (IDH1) and/or the oxidative pentose phosphate pathway (oxPPP). While ME1 and IDH1 extract hydrides from TCA-derived metabolites, the oxPPP diverts glucose-6-phospate from glycolysis to generate two equivalents of NADPH; one by G6PD, which catalyzes the first and committed step, and one by phosphogluconate-6-phosphate dehydrogenase (PGD).

The compounds described herein (e.g., compounds of Structural Formulas I-VI) exhibit dose-dependent effects consistent with decreased cellular activity of G6PD. Accordingly, provided herein is a method of modulating (e.g., inhibiting) the oxPPP, comprising contacting a cell with a compound described herein (e.g., a compound of any one of Structural Formulas I-VI), or a pharmaceutically acceptable salt thereof. In some embodiments, modulating the oxPPP comprises modulating (e.g., inhibiting) G6PD. In some embodiments, the cell is in a subject (e.g., a human). In some embodiments, the method is for treating a disease, condition or disorder described herein (e.g., cancer, malaria, autoimmune disease, inflammatory disease or condition, asthma).

Also provided herein is a method of treating a G6PD-mediated disease or condition in a subject in need thereof, comprising administering to the subject a therapeutically effective amount of a compound described herein (e.g., a compound of any one of Structural Formula I-VI), or a pharmaceutically acceptable salt thereof, or a composition described herein. As used herein, “glucose-6-phosphate dehydrogenase-mediated disease or condition” and “G6PD-mediated disease or condition” refer to any disorder or condition that is directly or indirectly regulated by G6PD. Examples of G6PD-mediated diseases or conditions include, but are not limited to, cancer, malaria, autoimmune diseases, inflammatory diseases and conditions and asthma, such as those described herein.

Also provided herein is a method of treating a disease or condition associated with G6PD activity or expression (e.g., aberrant G6PD activity or expression, such as upregulated G6PD activity or expression) in a subject in need thereof, comprising administering to the subject a therapeutically effective amount of a compound described herein (e.g., a compound of any one of Structural Formula I-VI), or a pharmaceutically acceptable salt thereof, or a composition described herein. Examples of diseases or conditions associated with G6PD activity or expression include, but are not limited to, cancer, malaria, autoimmune diseases, inflammatory diseases and conditions and asthma, such as those described herein.

“Treating,” as used herein, refers to taking steps to deliver a therapy to a subject, such as a mammal, in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“A therapeutically effective amount” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some embodiments, a subject is a human.

A compound described herein (e.g., a compound of any of Structural Formulas I-VI), or a pharmaceutically acceptable salt thereof, can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred mode of administration can vary depending on the particular compound chosen.

Certain methods further specify a delivery route such as intravenous, intramuscular, subcutaneous, rectal, intranasal, pulmonary, or oral.

A compound described herein, or a pharmaceutically acceptable salt thereof, can also be administered in combination with one or more other therapies (e.g., radiation therapy, a chemotherapy, such as a chemotherapeutic agent; an immunotherapy, such as an immunotherapeutic agent). When administered in a combination therapy, the compound, or pharmaceutically acceptable salt thereof, can be administered before, after or concurrently with the other therapy (e.g., radiation therapy, an additional agent(s)). When co-administered simultaneously (e.g., concurrently), the compound, or pharmaceutically acceptable salt thereof, and other therapy can be in separate formulations or the same formulation. Alternatively, the compound, or pharmaceutically acceptable salt thereof, and other therapy can be administered sequentially, as separate compositions, within an appropriate time frame as determined by a skilled clinician (e.g., a time sufficient to allow an overlap of the pharmaceutical effects of the therapies).

In some embodiments, a method described herein further comprises administering to the subject a therapeutically effective amount of an additional therapy (e.g., an additional therapeutic agent, such as a chemotherapeutic agent, an immunotherapeutic agent). In some embodiments (for example, wherein the cancer is colorectal cancer or ovarian cancer), the additional therapy is a platinum-based chemotherapy (e.g., oxaliplatin, cisplatin, carboplatin). In some embodiments (for example, wherein the cancer is FLT3 inhibitor-resistant AML), the additional therapy is a FLT3 inhibitor (e.g., sunitinib, sorafenib, midostaurin, lestaurtinib, ponatinib, crenolanib, quizartinib, gilteritinib). In some embodiments (for example, wherein the cancer is breast cancer), the additional therapy is a tyrosine kinase inhibitor (e.g., a FLT3 inhibitor, bosutinib, dasatinib, erlotinib, gefitinib, imatinib, lapatinib, nilotinib, pazopanib).

The oxPPP is important for the survival of cancer cells under low attachment conditions, including metastatic spread. Since metastasis is a dominant cause of cancer-related mortality, inhibitors of G6PD may, therefore, be useful as cancer therapeutics. Accordingly, provided herein is a method of treating cancer (e.g., by inhibiting metastasis of a cancer) in a subject in need thereof, comprising administering to the subject a therapeutically effective amount of a compound described herein (e.g., a compound of any one of Structural Formula I-VI), or a pharmaceutically acceptable salt thereof, or a composition described herein.

Such cancers include, for example, solid tumors and hematological malignancies (both adult and pediatric). Exemplary cancers include, but are not limited to, leukemia (including, but not limited to, acute lymphoblastic leukemia (ALL), acute myelogenous leukemia (AML) such as FLT3 inhibitor-resistant AML or AML with high mTORC1 expression and/or activity, chronic lymphocytic leukemia (CLL), chronic myelogenous leukemia (CML), acute monocytic leukemia (AMoL)), lymphoma (non-Hodgkin’s lymphoma or Hodgkin’s lymphoma), lung cancer (including non-small lung cancer), mesothelioma, breast cancer (including other solid tumors of the breast), liver cancer (including other solid tumors of the liver), colon or colorectal cancer (including other solid tumors of the colon and/or rectum), stomach cancer (including other solid tumors of the stomach), prostate cancer (including other solid tumors of the prostate), pancreatic cancer (including other solid tumors of the pancreas), ovarian cancer (including other solid tumors of the ovary), solid tumors of the uterus or female genital tract, bladder cancer (including other solid tumors of the bladder), head and neck cancers, glioblastoma and other brain tumors, and trophoblastic neoplasms. In certain embodiments, the cancer is a leukemia, preferably a T-cell leukemia. In certain embodiments, the cancer is a B-cell leukemia. In certain embodiments, the cancer is a lymphoma, preferably a T-cell lymphoma. In certain embodiments, the cancer is a B-cell lymphoma, for example, diffuse large B-cell lymphoma or a Burkitt lymphoma.

In certain embodiments, the cancer is selected from pediatric or adult leukemia including T-cell lymphoblastic leukemia, diffuse large B-cell lymphoma, acute myeloid leukemia, acute lymphoblastic leukemia, other lymphoma, other leukemia, solid tumors of the lung, non-small cell lung cancer, mesothelioma, solid tumors of the breast, colon cancer, liver cancer, stomach cancer, prostate cancer, pancreatic cancer, ovarian cancer, uterus and female genital tract cancer, bladder cancer, head and neck cancer, osteosarcoma, or trophoblastic neoplasms. In some embodiments, the cancer is colorectal cancer.

In some embodiments, the cancer is selected from T-cell lymphoblastic leukemia, diffuse large B-cell lymphoma, acute myeloid leukemia, acute lymphoblastic leukemia, non-small cell lung cancer, mesothelioma, a solid tumor of the lung, a solid tumor of the breast, colon cancer, liver cancer, stomach cancer, prostate cancer, pancreatic cancer, ovarian cancer, uterine or female genital tract cancer, bladder cancer, head and neck cancer, osteosarcoma, or a trophoblastic neoplasm.

Cancers associated with mutation(s) in the genes isocitrate dehydrogenase (IDH) 1 and/or 2 typically have less cytosolic NADPH available. Normal IDH1 produces NADPH, but mutant IDH1 actually consumes it. Without wishing to be bound by any particular theory, it is believed that such cancers may be more sensitive to G6PD inhibition. Accordingly, in some embodiments, the cancer is a cancer associated with mutation(s) in the genes isocitrate dehydrogenase (IDH) 1 and/or 2 (e.g., IDH1), such as AML or glioblastoma.

In some embodiments, the cancer is a cancer associated with accumulating NF-E2-related factor 2 (NRF2) (e.g., NRF2-driven breast cancer metastasis).

Other examples of cancer treatable according to the methods described herein include Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Anal Cancer; Appendix Cancer; Astrocytomas, Childhood; Atypical Teratoid/Rhabdoid Tumor, Childhood, Central Nervous System; Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sézary Syndrome); Ductal Carcinoma In Situ (DCIS); Embryonal Tumors, Central Nervous System, Childhood; Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sézary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sèzary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated in accordance with the methods described herein. In some embodiments, the cancer is a metastatic cancer.

Humans possessing hypomorphic mutations in G6PD generally possess resistance to certain strains of malaria due to the critical role G6PD plays in red blood cells, in which the malaria parasite spends part of its life cycle. Importantly, individuals with these mutations present with few other associated symptoms. Thus, inhibitors of G6PD may also serve as important prophylactic or acute treatments for individuals sick with malaria. Accordingly, also provided herein is a method of treating malaria in a subject in need thereof, comprising administering to the subject a therapeutically effective amount of a compound described herein (e.g., a compound of any one of Structural Formula I-VI), or a pharmaceutically acceptable salt thereof, or a composition described herein.

G6PD is also a validated target in Trypanosoma brucei, a parasite causing Human African Trypanosomiasis, and is also implicated in T. cruzi, a parasite causing Chagas disease, where inhibition of G6PD has been shown to kill the parasite in vitro. Mercaldi, G.F., et al., Journal of Biomolecular Screening 2014, Vol. 19(10), 1362-1371, the entire content of which is incorporated herein by reference.

Accordingly, also provided herein is a method of treating a parasitic infection in a subject in need thereof, comprising administering to the subject a therapeutically effective amount of a compound described herein (e.g., a compound of any one of Structural Formulas I-VI), or a pharmaceutically acceptable salt thereof, or a composition described herein. In some embodiments, the parasitic infection is trypanosomiasis (e.g., Human African Trypanosomiasis, Chagas disease). In some embodiments, the parasitic infection is malaria.

It has also been discovered that lymphocytes are especially reliant on G6PD activity for maintaining their NADPH pools. Furthermore, G6PD inhibition disrupts cytokine production in activated T cells, implying that G6PD inhibitors may serve as useful immunomodulatory agents. Accordingly, also provided herein is a method of treating an autoimmune disease, comprising administering to the subject a therapeutically effective amount of a compound described herein (e.g., a compound of any one of Structural Formula I-VI), or a pharmaceutically acceptable salt thereof, or a composition described herein.

Autoimmune diseases include, but are not limited to, fibrosis, rheumatoid arthritis, multiple sclerosis, systemic lupus erythematosus, type 1 diabetes mellitus, Guillain-Barre syndrome, chronic inflammatory demyelinating polyneuropathy, Graves’ disease, Hashimoto’s thyroiditis, myasthenia gravis, inflammatory bowel disease (e.g., Crohn’s disease or ulcerative colitis), polymyositis, dermatomyositis, inflammatory myositis, ankylosing spondolytis, ulcerative colitis, psoriasis, vasculitis, Sjogren’s disease and transplant rejection. In some embodiments, an autoimmune disease is rheumatoid arthritis, Crohn’s disease, ulcerative colitis, psoriasis, vasculitis, multiple sclerosis, Sjogren’s disease, systemic lupus erythematosus or transplant rejection. In some embodiments, an autoimmune disease is fibrosis.

In addition, G6PD is thought to be critical for the inflammatory response in leukocytes, especially those lineages that produce an “oxidative burst” (e.g., neutrophils, macrophages). Thus, inhibitors of G6PD may serve as agents for modulating conditions associated with overactive inflammatory responses. Accordingly, also provided herein is a method of treating an inflammatory disease or condition in a subject in need thereof, comprising administering to the subject a therapeutically effective amount of a compound described herein (e.g., a compound of any one of Structural Formula I-VI), or a pharmaceutically acceptable salt thereof, or a composition described herein.

Inflammatory diseases and conditions include, but are not limited to, multiple sclerosis, Goodpasture syndrome, psoriasis, ankylosing spondylitis, antiphospholipid antibody syndrome, gout, arthritis (e.g., rheumatoid arthritis), myositis, scleroderma, Sjogren’s syndrome, systemic lupus erythematosus and vasculitis.

It has also been discovered that the compounds described herein can inhibit both pro-inflammatory T-cells and neutrophils. Accordingly, also provided herein is a method of treating asthma, comprising administering to the subject a therapeutically effective amount of a compound described herein (e.g., a compound of any one of Structural Formula I-VI), or a pharmaceutically acceptable salt thereof, or a composition described herein.

Also provided herein is a method of inhibiting oxidative burst, comprising contacting a cell that produces oxidative burst (e.g., a neutrophil, macrophage) with a compound described herein (e.g., a compound of any one of Structural Formula I-VI), or a pharmaceutically acceptable salt thereof, or a composition described herein, thereby inhibiting oxidative burst. Also provided is a method of inhibiting oxidative burst in a subject in need thereof (e.g., a subject having asthma or an inflammatory disease or condition), the method comprising administering to the subject a therapeutically effective amount of a compound described herein (e.g., a compound of any one of Structural Formula I-VI), or a pharmaceutically acceptable salt thereof, or a composition described herein.

Also provided herein is a method of inhibiting an immune response in a subject in need thereof (e.g., a subject having asthma or an inflammatory disease or condition), the method comprising administering to the subject a therapeutically effective amount of a compound described herein (e.g., a compound of any one of Structural Formula I-VI), or a pharmaceutically acceptable salt thereof, or a composition described herein.

Also provided herein is a method of treating a disease or condition associated with dysregulation of the circadian clock in a subject in need thereof (e.g., a subject having cancer or a metabolic disorder associated with dysregulation of the circadian clock), the method comprising administering to the subject a therapeutically effective amount of a compound described herein (e.g., a compound of any one of Structural Formula I-VI), or a pharmaceutically acceptable salt thereof, or a composition described herein.

Also provided herein is a method of treating a disease or condition associated with a mutation in G6PD (e.g., a mutation resulting in decreased G6PD activity, a mutation resulting in increased G6PD activity) in a subject in need thereof, comprising administering to the subject (e.g., a subject having T-ALL) a therapeutically effective amount of a compound described herein (e.g., a compound of any one of Structural Formula I-VI), or a pharmaceutically acceptable salt thereof, or a composition described herein.

In addition to being therapeutic agents, the compounds described herein (e.g., a compound of any one of Structural Formulas I-VI), or a pharmaceutically acceptable salt thereof, may be useful as research tools, e.g., as a tool compound for altering G6PD activity in cells, studying the oxPPP.

### Assays

The cellular activity of most reported inhibitors of G6PD has been evaluated using non-specific assays, including monitoring treated cells for increases in oxidative stress or decreased viability/proliferation upon compound exposure. Though these manifestations can certainly result from G6PD inhibition in some cases, they can also arise from compound interactions with cellular targets unrelated to G6PD (e.g., off-target effects). To convincingly evaluate whether or not a compound engages with G6PD in cells, assays that specifically monitor the G6PD reaction are required.

The following four cellular assays more directly monitor G6PD activity in intact cells. These assays can broadly be divided into two categories: a) those that monitor the carbohydrate product (6-pg) of the G6PD reaction, and b) those that monitor the NADPH produced by the G6PD reaction.

Direct Monitoring of 6-pg in cells. The metabolite 6-phosphogluconate (6-pg) is a downstream product of the G6PD reaction, and is believed to be made predominantly by the oxidative pentose phosphate pathway in mammalian cells. Though the direct product of G6PD is the lactone form of 6-pg (6-phosphogluconolactone), this product is believed to readily hydrolyze to 6-pg under cellular conditions, and is therefore difficult to detect. Thus, to directly monitor the G6PD reaction, methods for measuring 6-pg levels in intact cells were developed. During the development of these methods, it was challenging to identify a cell line that possessed detectable levels of 6-pg at baseline (i.e., prior to G6PD inhibition). Ultimately, this problem was solved in two ways: i) by identifying a cell line with naturally high 6-pg levels (HepG2 cells), and ii) generating a clonally derived cell line that possess a hypomorphic PGD enzyme, the main enzyme that consumes 6-pg (HCT116-mPgd cells).

Accordingly, one embodiment is a method of identifying a G6PD inhibitor, comprising contacting a cell (e.g., a mammalian cell) with enhanced 6-pg levels (e.g., a HepG2 cell) with a compound (e.g., a compound described herein, such as a compound of any one of Structural Formulas I-VI, or a pharmaceutically acceptable salt thereof), and detecting (e.g., by LC-MS) 6-pg in the cell, wherein a decrease in 6-pg compared to an appropriate control indicates the compound is a G6PD inhibitor. A person skilled in the art will be able to determine which cells have enhanced 6-pg levels in view of this disclosure.

One embodiment is a method of identifying a G6PD inhibitor, comprising contacting a cell (e.g., a mammalian cell, such as a HCT116 cell) with decreased PGD activity with a compound (e.g., a compound described herein, such as a compound of any one of Structural Formulas I-VI, or a pharmaceutically acceptable salt thereof), and detecting (e.g., by LC-MS) 6-pg in the cell, wherein a decrease in 6-pg compared to an appropriate control indicates the compound is a G6PD inhibitor. In some embodiments, the cell possesses a hypomorphic mutation in Pgd that results in decreased PGD expression and decreased PGD activity. For example, CRISPR-Cas9 gene editing can be used to generate a cell line that possesses lower PGD expression, leading to a build-up of 6-pg (the substrate of PGD), improved detection of 6-pg, and a wider dynamic range for monitoring cellular target engagement. A cell with decreased PGD activity can also be generated and/or provided, for example, using RNA interference, by inhibiting PGD (e.g., with an inhibitor) and/or identifying a cell or cell line having a mutation in PGD that lowers its expression and/or activity. A person skilled in the art will be able to determine which cells have decreased PGD activity, and how to provide such cells in view of this disclosure.

Direct Monitoring of NADPH Produced by the G6PD Reaction. NADPH is a high-energy cofactor required for several crucial cellular functions, most notably reductive biosynthesis and maintenance of antioxidant defenses. NADPH production is compartmentalized, and occurs through reduction of its redox partner NADP⁺. G6PD is one of the main enzymes that generates NADPH, and catalyzes the transfer of electrons (in the form of hydride) from glucose-6-phosphate to NADP⁺. By installing a deuterium at the “one” position of glucose (1-²H-glucose), the transfer of that deuterium to the active hydride of NADPH via glucose-6-phosphate can be specifically monitored. Also, a major use of cytosolic NADPH is fat synthesis. Transfer of ²H from glucose via NADPH into free palmitate (C16:0) requires two NADPH per two-carbon unit addition during its synthesis.

Accordingly, one embodiment is a method of identifying a G6PD inhibitor, comprising contacting a cell cultured in 1-²H-glucose with a compound (e.g., a compound described herein, such as a compound of any one of Structural Formulas I-VI, or a pharmaceutically acceptable salt thereof), and detecting NADP²H or ²H-labeled palmitate (e.g., by LC-MS) in the cell, wherein a decrease in NADP²H or ²H-labeled palmitate, respectively, compared to an appropriate control indicates the compound is a G6PD inhibitor.

In certain cells, especially the immortalized T cell lineage known as Jurkat cells, the oxPPP appears to be the dominant source of NADPH, with inhibition of G6PD leading to a dramatic decrease of NADPH (and a concurrent increase in NADP+). Thus, monitoring total NADPH and NADP+ levels in these cells is another approach for monitoring cellular G6PD activity.

Accordingly, one embodiment is a method of identifying a G6PD inhibitor, comprising contacting a cell wherein the oxPPP is the dominant source of NADPH (e.g., a Jurkat cell) with a compound (e.g., a compound described herein, such as a compound of any one of Structural Formulas I-VI, or a pharmaceutically acceptable salt thereof), and detecting NADP²H or ²H-labeled palmitate (e.g., by LC-MS) in the cell, wherein the cell is cultured in 1-²H-glucose; and a decrease in NADP²H or ²H-labeled palmitate, respectively, compared to an appropriate control indicates the compound is a G6PD inhibitor. In a cell wherein the oxPPP is the dominant source of NADPH in the cell, the oxPPP can be the source of greater than 50%, 60%, 70%, 75%, 80%, 85%, 90%, 95%, 96%, 97, 98% or 99% of the NADPH in the cell.

Appropriate controls for the assays described herein will be known to those of ordinary skill in the art, and include those described herein, for example, in the Exemplification.

### EXEMPLIFICATION

### Abstract

Glucose is catabolized by two fundamental pathways, glycolysis, to make ATP, and the oxidative pentose pathway, to make NADPH. The first step of the oxidative pentose phosphate pathway is catalyzed by the enzyme glucose-6-phosphate dehydrogenase (G6PD). Metabolite reporter and deuterium tracer assays were developed, and used to monitor cellular G6PD activity. A widely-cited G6PD antagonist, dehydroepiandrosterone (DHEA), failed to inhibit G6PD in cells. As described herein, a small molecule compound (G6PDi-1) was found to effectively inhibit G6PD. Across a range of cultured cells, G6PDi-1 depleted NADPH most strongly in lymphocytes. In T cells, G6PDi-1 did not block initial activation or proliferation, but nearly completely ablated cytokine production. These data suggest that G6PDi-1 is a cell-active small molecule tool for oxidative pentose phosphate pathway inhibition, and that G6PD is a pharmacological target for modulating immune response.

### Introduction

Across all forms of life, the redox cofactor NADPH donates high-energy electrons for reductive biosynthesis and antioxidant defense. The critical nature of these processes requires effective maintenance of the levels of NADPH and its redox partner NADP+. In the cytosol of mammalian cells, reduction of NADP+ to NADPH mainly occurs via three routes: malic enzyme 1 (ME1), isocitrate dehydrogenase 1 (IDH1), and the oxidative pentose phosphate pathway (oxPPP). While ME1 and IDH1 extract hydrides from TCA-derived metabolites, the oxPPP diverts glucose-6-phospate from glycolysis to generate two equivalents of NADPH; one by G6PD, which catalyzes the first and committed step, and one by phosphogluconate-6-phosphate dehydrogenase (PGD).

G6PD is ubiquitously expressed in mammalian tissues, with highest expression in immune cells and testes. It is also often upregulated in tumors. Genetically, G6PD knockout mice are inviable. Nevertheless, G6PD hypomorphic alleles are common in humans, affecting approximately one in 20 people worldwide. These mutations provide protection from malaria, but sensitize mature red blood cells (RBCs) to oxidative stressors. The vulnerability of RBCs to mutant G6PD may reflect RBCs’ lack of mitochondria and thus inability to endogenously produce the substrates of ME1 or IDH1. Alternatively, it may reflect RBCs′ lack of nuclei and thus inability to replace the mutant G6PD protein as the cells age. In other tissues, the function of G6PD is less investigated. Using a genetic approach, it was recently shown that cancer cell lines lacking G6PD have elevated NADP+ levels, but are nevertheless able to proliferate and maintain NADPH pools through compensatory ME1 and/or IDH1 flux. Whether non-transformed cells are similarly flexible remains unclear.

Potent and selective small molecule inhibitors are useful tools for studying the function of metabolic enzymes. To date, several small molecule inhibitors of G6PD have been described, most notably the steroid derivative dehydroepiandrosterone (DHEA). First reported in 1960, DHEA binds mammalian G6PD uncompetitively against both reaction substrates. Since then, DHEA and its derivatives have been employed as G6PD inhibitors in hundreds of studies, including a variety of in vitro and in vivo cancer settings where they display anti-proliferative activity. However, these readouts of cellular activity are indirect, and it has been proposed that the effects of DHEA may arise from mechanisms other than G6PD inhibition.

To properly evaluate cellular target engagement, it is important to employ assays that specifically monitor the reaction of interest. However, developing assays that monitor NADPH-producing reactions can be particularly challenging, since NADPH is difficult to measure and is produced by multiple pathways (where inhibition of one can be masked by compensatory production from others).

Described herein is the development of G6PD cellular target engagement assays, and use of the assays to show that DHEA, even at high doses, fails to inhibit G6PD in cells. A non-steroidal small molecule inhibitor of G6PD, G6PDi-1, is then identified. G6PDi-1 demonstrates on-target, reversible cellular activity against G6PD. Utilization of G6PDi-1 across a wide range of mammalian cells revealed that cells of T cell lineage are unexpectedly reliant on G6PD, and, upon G6PDi-1 treatment, cannot maintain NADPH levels or cytokine production.

### Results

The results described herein are also reported in Ghergurovich, et al., Nat Chem Biol. 2020 Jul; 16(7):731-739, the entire content of which is incorporated herein by reference.

### Cell-based Assays of G6PD Activity Reveal Lack of Target Engagement by DHEA

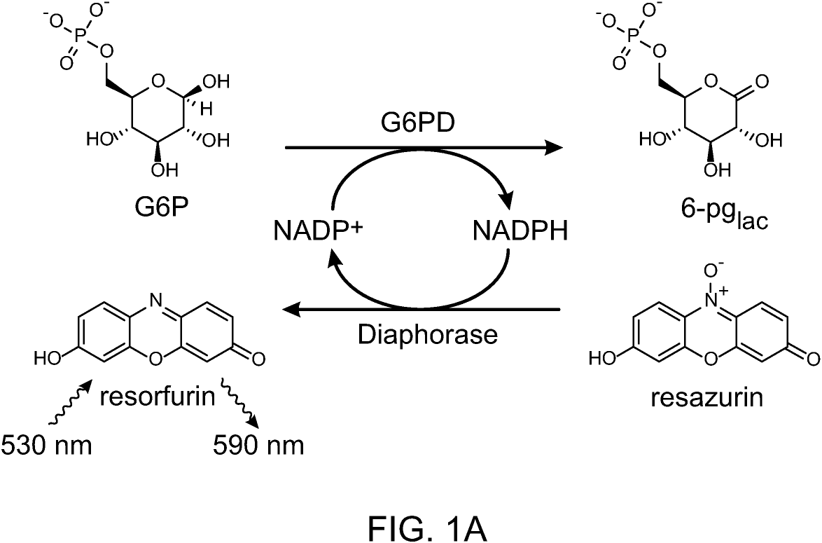

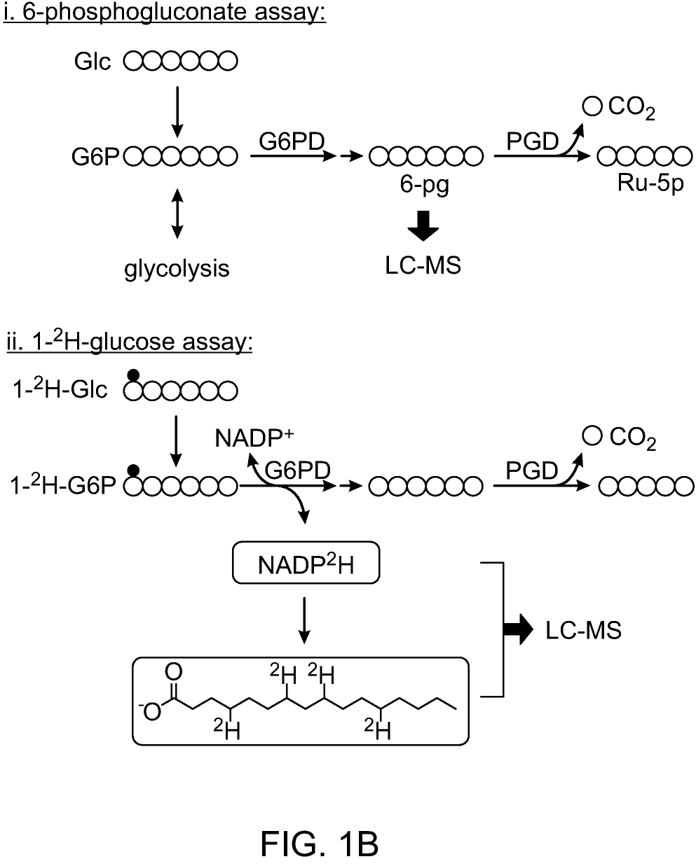

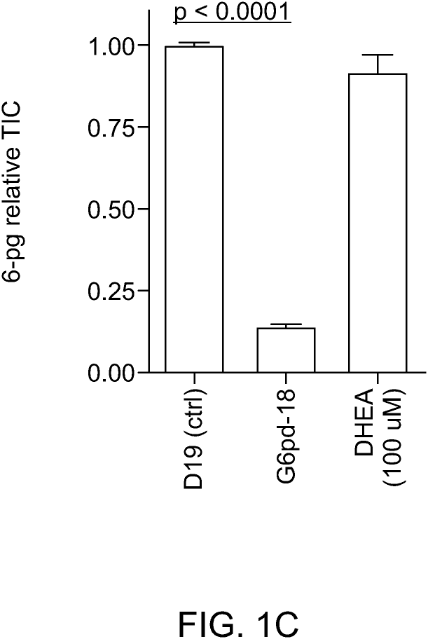

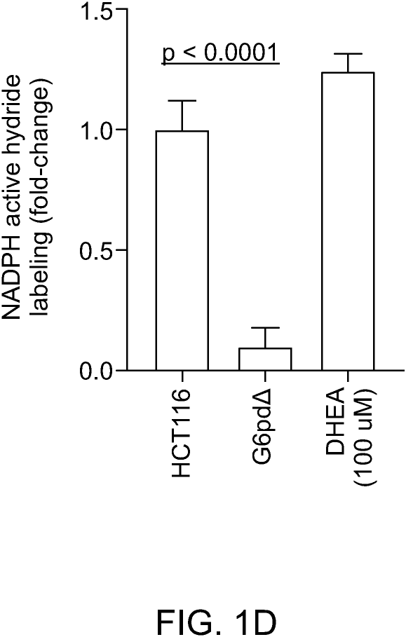

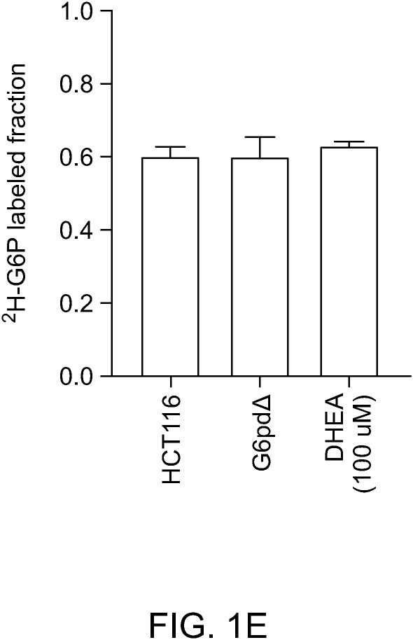

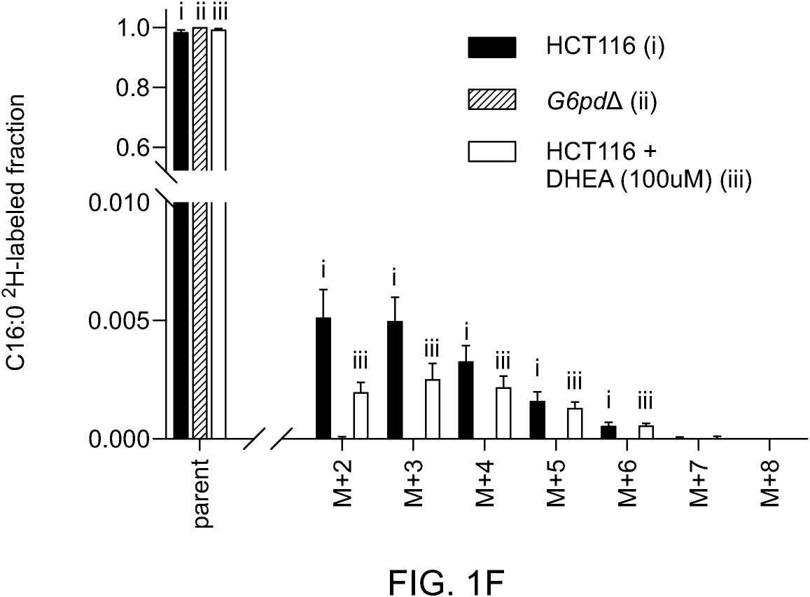

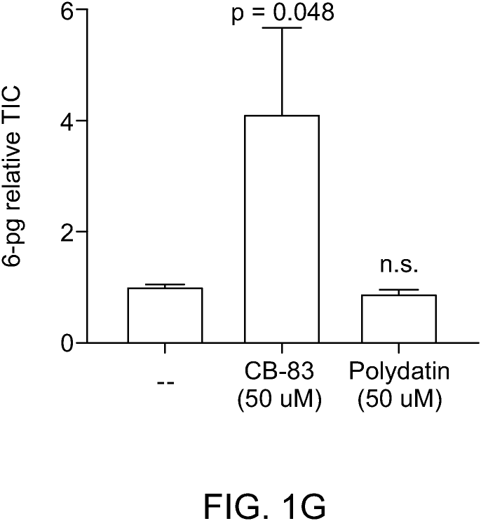

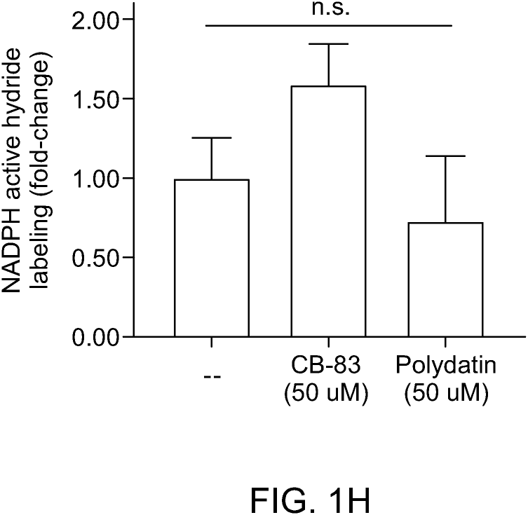

To examine the biochemical activity of G6PD, a coupled enzymatic assay using recombinant human enzyme was established (FIG. 1A). Consistent with prior reports, DHEA demonstrated dose-dependent inhibition of G6PD, with a calculated half-maximal inhibitory constant (IC50) of approximately 9.5 µM.

To assess whether DHEA effectively targets G6PD also in cells, metabolomics of clonally isolated G6PD knockout cells (G6pdΔ) were compared with parental HCT116 cells treated with high dose DHEA (100 µM). DHEA treatment did not mirror G6PD knockout, instead producing a different metabolic signature, raising the possibility that DHEA may not effectively target cellular G6PD.

To explore this possibility further, more specific assays for evaluating G6PD cellular target engagement were developed. Direct monitoring of 6-phosphogluconolactone, the immediate carbohydrate product of the G6PD reaction, proved challenging, likely due to its tendency to hydrolyze. Some cell lines, however, possessed measurable levels of the resulting product, 6-phosphogluconate (6-pg). These included the hepatocellular carcinoma line, HepG2, which was selected as the primary cell for direct 6-pg monitoring. Consistent with the oxPPP being the primary source of 6-pg in these cells, G6PD knockout led to nearly complete 6-pg loss (FIGS. 1B and 1C). In contrast, DHEA (100 µM) did not impact the 6-pg level (FIG. 1C).

Next, G6PD mediated hydride transfer to NADPH was directly monitored. Specifically, transfer of deuterium from 1-²H-glucose, via glucose-6-phosphate, to the NADPH’s active hydride was monitored (FIG. 1B). Consistent with this labeling arising primarily from the G6PD reaction, G6PD-knockout cells demonstrated a nearly complete loss of active hydride labeling (FIG. 1D). The impacted step was G6PD, as no change in substrate (G6P) labeling was observed (FIG. 1E). A major use of cytosolic NADPH is fat synthesis. Transfer of ²H from glucose via NADPH into palmitate (C16:0), which requires two NADPH per 2-carbon unit addition during its synthesis, was quantified (FIG. 1B). Near complete loss of labeling into C16:0 from 1-²H-glucose (FIG. 1F) was observed in G6pdΔ cells. DHEA, however, did not decrease either NADPH active hydride labeling (FIG. 1D) or C16:0 labeling (FIG. 1F). Thus, DHEA does not robustly inhibit cellular G6PD.

To evaluate other purported inhibitors of G6PD, two recently identified small molecules, CB-83 and polydatin, were obtained. Like DHEA, both CB-83 and polydatin display anti-proliferative effects against transformed cells, but direct evidence of cellular G6PD inhibition was lacking. At a dose higher than that reported to impair cell growth, polydatin failed to decrease 6-pg levels (FIG. 1G) or NADPH active hydride labeling (FIG. 1H), consistent with lack of cellular target engagement of G6PD. Although individual experimental results were quite variable, CB-83 appeared to augment G6PD activity (FIGS. 1G and 1H). This could potentially reflect CB-83 activating the oxPPP by inducing oxidative stress. Despite this complexity, like DHEA, these compounds do not appear to be cell-active G6PD inhibitors.

### G6PDi-1, a Non-steroidal, Cell Active Inhibitor of G6PD

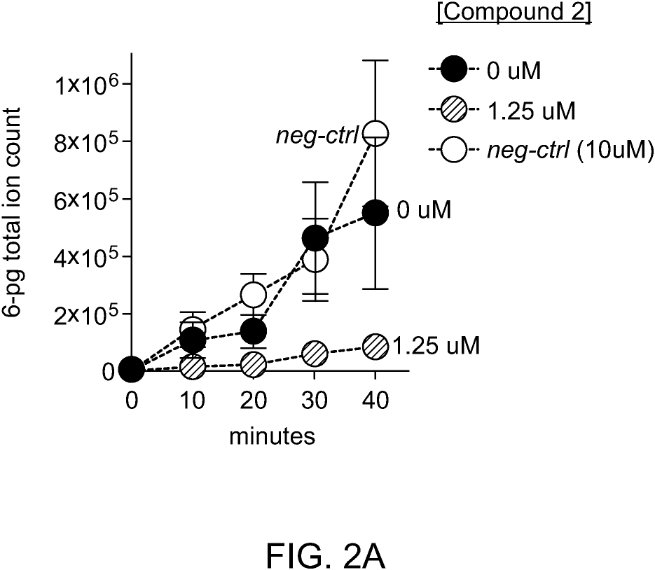

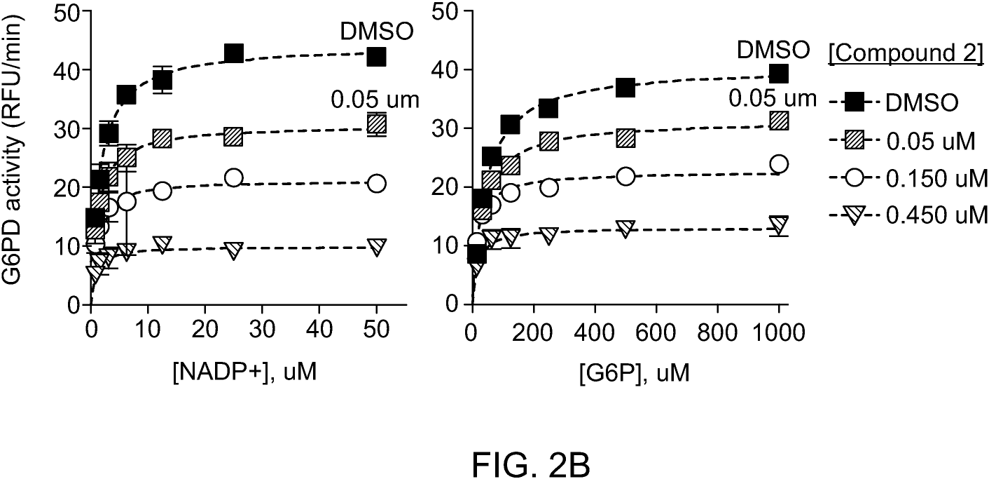

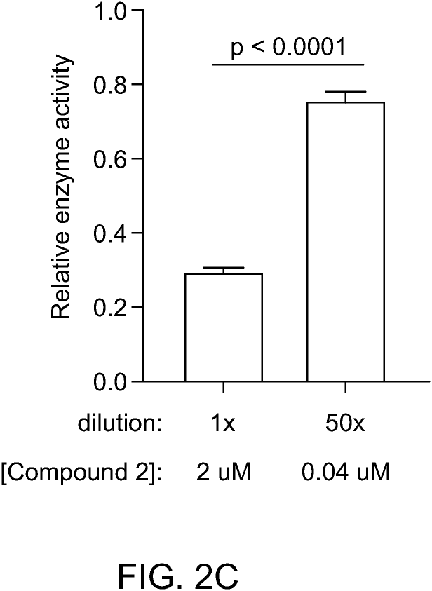

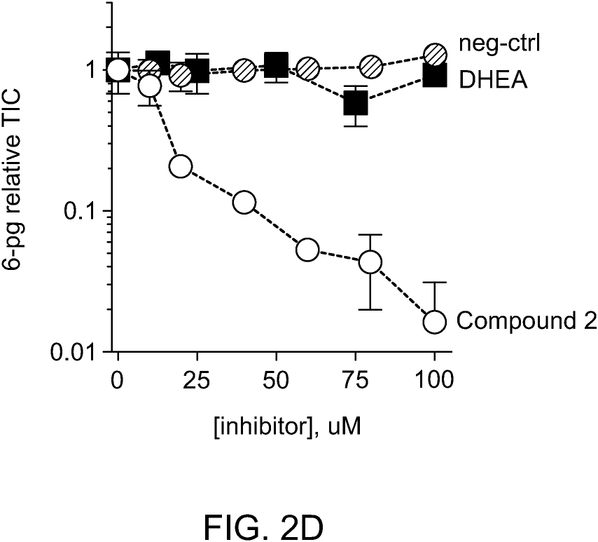

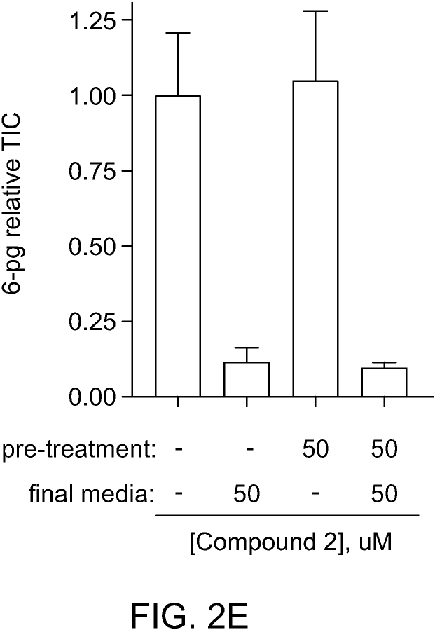

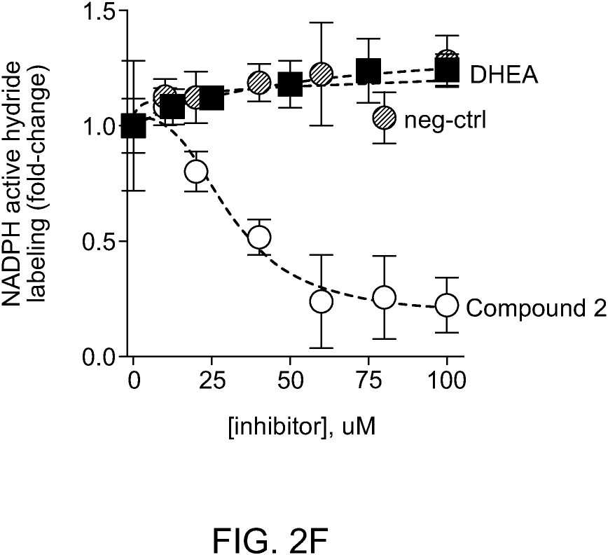

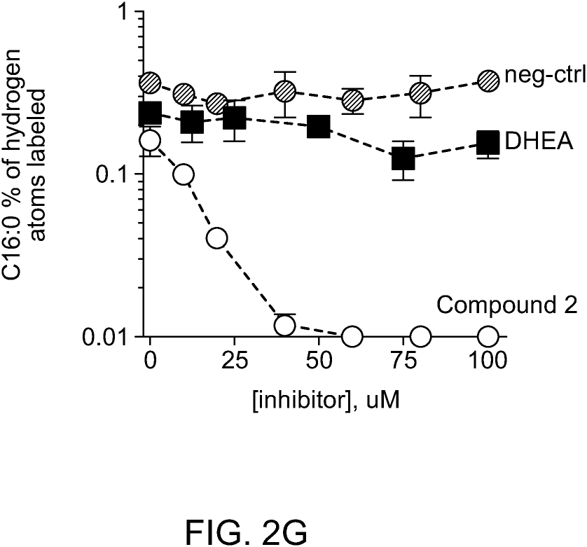

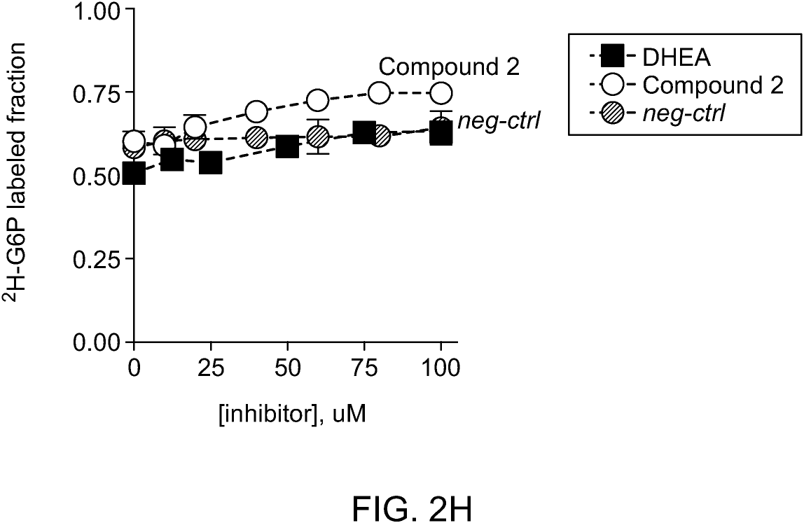

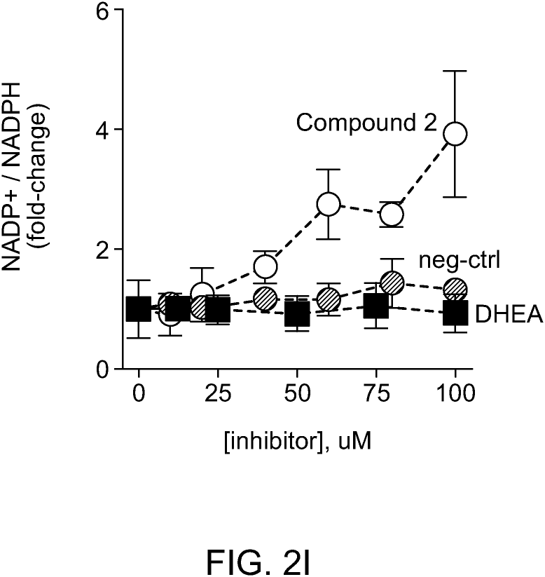

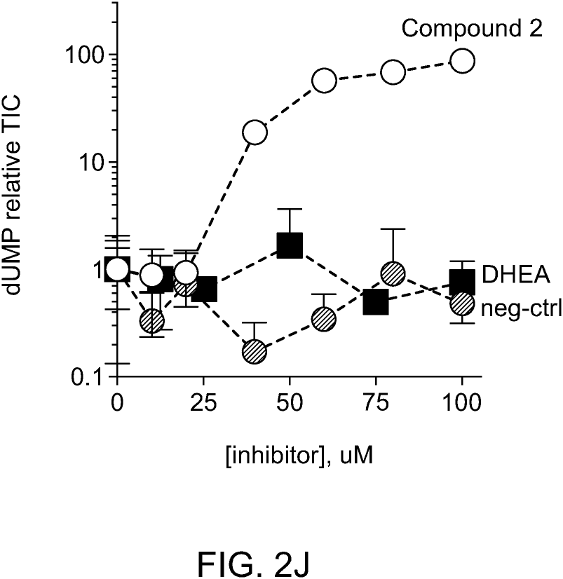

Successive rounds of synthesis and screening led to Compound 2 (G6PDi-1), a sub-micromolar inhibitor of human G6PD (IC₅₀ =0.07 µM). Additionally, a structural analogue (designated neg-ctrl) that lacked any activity against G6PD was identified to serve as a negative control compound. In vitro activities were verified in an orthogonal, LC-MS assay that monitors 6-pg production by recombinant human G6PD. Again, Compound 2 demonstrated in vitro inhibition of G6PD activity, while high dose neg-ctrl did not (FIG. 2A). Follow up competition assays against both substrates (FIG. 2B) and in vitro dilution experiments (FIG. 2C) showed Compound 2 binds to G6PD non-competitively and reversibly. Cellular thermal shift assay (CETSA) using HepG2 lysates demonstrated significant thermal stabilization of G6PD by G6PDi-1, but not DHEA up to 56° C. These data collectively support a reversible direct physical interaction between G6PDi-1 and G6PD at an allosteric site, with G6PDi-1 binding inhibiting enzyme catalytic activity.

To investigate cellular target engagement, Compound 2 was evaluated in established target engagement assays. In HepG2 cells, treatment with Compound 2, but not neg-ctrl or DHEA, led to a dose-dependent decrease in 6-pg levels (IC₅₀ approximately 13 µM; FIG. 2D). Consistent with this effect arising from reversible binding of G6PD, 6-pg levels completely recovered within 2 hours of removing the inhibitor (FIG. 2E). Additionally, treatment of HCT116 cells with Compound 2, but not neg-ctrl or DHEA, led to a dose-dependent decrease in ²H transfer from 1-²H-glucose to NADPH’s active hydride (IC₅₀ approximately 31 µM; FIG. 2F) and downstream product C16:0 (FIG. 2G). The impacted step was G6PD, as no change in G6P labeling was observed (FIG. 2H). In addition, as expected for G6PD inhibition, dose-dependent increases in NADP/NADPH were observed (FIG. 2I). Moreover, consistent with recent findings where genetic G6PD depletion led to impaired folate metabolism and associated blockade of thymidylate synthase with buildup of dUMP, treatment with Compound 2, but not neg-ctrl or DHEA, led to dose-dependent increases in dUMP (FIG. 2J).

It has been established that epithelial cells undergoing matrix detachment are subjected to increased levels of oxidative stress, and are in turn dependent on oxPPP activity for survival. It was observed that colony formation of G6pdΔ cells was dramatically impaired upon treatment with Compound G6PDi-1. Consistent with G6PDi-1 possessing cellular G6PD activity, a dose-dependent decrease in colony formation was observed with G6PDi-1, but not neg-ctrl, an effect that was rescued by exogenous antioxidants, such as N-acetylcysteine.

Taken together, these data show that Compound 2 is a cell-active G6PD inhibitor

### G6PDi-1 Reveals T Cell Dependence on oxPPP

It was recently established that transformed cells can maintain NADPH levels in the face of G6pd loss by using malic enzyme 1 (ME1) and/or isocitrate dehydrogenase 1 (1DH1) to make NADPH. To evaluate the potential for different cells to acutely compensate for G6PD inhibition, a diversity of primary and transformed cell types were treated with G6PDi-1, reasoning that cells reliant on the oxPPP would be unable to maintain their NADPH pools. RBCs were fairly sensitive to G6PDi-1. Surprisingly, T cell lineages were substantially more strongly affected by G6PDi-1, manifesting a >10-fold decrease in NADPH, accompanied by a 5-10-fold increase in NADP+ (FIG. 3A). Thus, T cells appear to be particularly dependent on the oxPPP for maintaining their NADPH pools.

Since T cell activation involves substantial metabolic rewiring, whether an activation-driven metabolic program determined T cell dependency on the oxPPP was investigated. To this end, naïve CD8+ T cells were isolated from mouse spleen and either maintained in the naïve state by culturing them with IL7, or activated with plate-bound αCD3/αCD28 and IL-2. Upon activation, an increase in G6PD protein was observed, which began within 8 hours and became prominent by 24 hours (FIG. 3B). In line with this observation, absolute oxPPP flux as measured using radioactive CO₂ capture increased by greater than 10-fold upon activation (FIG. 3C).

Consistent with the inability of T cells to maintain NADPH using compensatory pathways, neither naïve nor activated CD8+ T cells possessed substantial ME1 or IDH1 (FIG. 3B). To complement these enzyme abundance measurements, ²H-tracing was used to assess the relative contribution from ME1, IDH1 and the oxPPP to cytosolic NADPH. Using a combination of five tracers (FIGS. 3D and 3E), it was found that the oxPPP accounts for nearly all cytosolic NADPH production in CD8+ and CD4+ T cells activated with αCD3/αCD28 and IL-2, but not in naïve CD4+ and CD8+ T cells maintained in IL-7 supplemented media (FIGS. 3F and 3G).

To examine further the impact of G6PDi-1, mouse CD8+ and CD4+ T cells at day 4-5 post-activation were treated with increasing G6PDi-1 in the presence of 1-²H-glucose. G6PDi-1 (10 µM) completely blocked ²H transfer from glucose to NADPH (FIGS. 3H and 3I) and decreased NADPH and 6-pg levels (FIG. 3J). Similarly, treatment with G6PDi-1 blocked absolute oxPPP flux (FIG. 3K). NADPH, NADP+ and 6-pg levels were restored within 2 hours of removing the inhibitor (FIGS. 3L and 3M). The effects on NADPH labeling (from 1-²H-glucose), and NADP+, NADPH and 6-pg levels occurred within 10 minutes of G6PDi-1 treatment (FIG. 3N). Absolute quantitation of NADP+ and NADPH revealed that the decrease in NADPH concentration induced by G6PDi-1 is matched by an increase in NADP+ concentration, with the total NADP(H) remaining around approximately 200 µM (FIG. 3O). Collectively, these data confirm that G6PDi-1 is a rapid, reversible G6PD inhibitor that increases the NADP+/NADPH ratio in T cells.

To assess the specificity of the metabolic effects of G6PDi-1, untargeted metabolomics were performed on CD8+ T cells. The greatest metabolite concentration change occurred directly in the substrates and products of G6PD (NADPH, NADP+, 6-pg) and folate metabolites known to be perturbed by G6PD activity loss (dUMP, GAR) (FIG. 3P). Thus, G6PDi-1 has clean on-target activity in T cells. Isotope tracing with [U-¹³C]-glucose and [U-¹³C]-glutamine revealed that G6PDi-1 decreased the glucose contribution to TCA cycle (with a corresponding increase in glutamine contribution). In addition, fatty acid synthesis, a major consumer of cytosolic NADPH, was nearly completely ablated.

Consistent with the importance of NADPH in controlling oxidative stress, G6PDi-1 treatment elevated reactive oxygen species in both CD8+ and CD4+ T cells (FIGS. 3Q and 3R). These effects were largely rescued by N-acetyl cysteine (FIGS. 3S and 3T).

A transgenic mouse strain that over-expresses human G6PD (G6PD-Tg) has been reported (FIG. 3U). To validate the dependence of T cell NADPH pools on G6PD, CD8⁺ T cells from G6PD-Tg mice and littermate controls at day 4-5 post-activation were treated with increasing doses of G6PDi-1. Strikingly, G6PD overexpression markedly shifted the dose response to G6PDi-1, rescuing its effects on NADPH and NADP⁺ (FIGS. 3J and 3K). Thus, G6PDi-1 modulates T cell NADPH by inhibiting the catalytic activity of G6PD, with introduction of exogenous G6PD activity rescuing T cell redox state.

### G6PDi-1 Blocks T Cell Cytokine Secretion

The functional consequences of oxPPP inhibition were next explored using G6PDi-1 in T cells. To test the effect of G6PDi-1 on activation and proliferation, naïve CD8+ T cells were isolated from spleen and activated in vitro with plate-bound αCD3/αCD28 and IL-2. Activation was evaluated by flow cytometry analysis of surface markers CD69 (levels rapidly rise upon activation) and CD25 (usually peaking at 24-48 hours post-activation), and cell size, which increases over the first 24 hours post-activation. To quantify proliferation, naïve cells were stained with Crystal Trace Violet (CTV) and dye dilution was measured by flow cytometry at day 4 post-activation. As expected by the late upregulation of oxPPP during activation (FIG. 3B), G6PDi-1 did not alter the normal upregulation of activation markers or activation-dependent increase in cell size (FIG. 4A). More surprisingly, G6PDi-1 had a minimal effect in activation-dependent proliferation (FIGS. 4B and 4C) and viability (FIG. 4D). G6PDi-1 had also a minimal effect on the proliferation of CD4⁺ T cells.

To assess the effect of G6PD inhibition in T cell function, active CD8+ or CD4+ cells were stimulated with PMA and ionomycin in the presence of increasing doses of G6PDi-1. As a control, classically activated macrophages (MΦ-1) were included, as they derive substantial amounts of NADPH from the oxPPP (FIG. 4E), but their NADPH pool size is resistant to G6PDi treatment (FIG. 3A). Cytokine production was monitored by intracellular flow cytometry. Strikingly, while minimally affecting macrophage cytokine production, G6PD inhibition blocked cytokine production in both CD8+ and CD4+ T cells (FIGS. 4F-4H).

Proper T cell activation requires ROS signaling while avoiding ROS toxicity. Accordingly, rescue of CD8+ T cell cytokine secretion was attempted with the antioxidant N-acetyl-cysteine (FIG. 4I) or by providing an external source of peroxide/superoxide (FIG. 4J), but neither was effective. To confirm that the defect is at the level of signaling, rather than protein synthesis, IFNγ mRNA was examined, finding that its levels were also decreased (FIG. 4K). Indeed, signaling during the first hour after restimulation seemed to be particularly important, as delayed addition of G6PDi-1 enabled substantial cytokine production to occur (FIG. 4L). Restoration of intracellular NADPH levels by moderate overexpression of human G6PD protein decreased sensitivity to G6PDi-1 and partially normalized both protein and mRNA levels upon G6PDi-1 addition (FIGS. 4M and 4N). Thus, G6PD activity is required to maintain proper NADP/NADPH homeostasis in T cells, in a manner that is not readily compensated by generic oxidant or antioxidant, and loss of such homeostasis inhibits T cell function.

Whether G6PD inhibition impacted the development, proliferation or suppressor function of CD4⁺ regulatory T cells (Treg) was next evaluated. Stimulation with CD3/CD28 in the presence of TGF-β resulted in Foxp3⁺ cells, whose formation and proliferation were unaffected by G6PDi-1. Similarly, CD4⁺CD25⁺ Tregs proliferated and were effective in suppressing the proliferation of conventional CD4⁺ CD25⁻ T cells irrespective of G6PDi-1 treatment. Collectively, these data show that, without overtly impacting proliferation or suppressor function, G6PDi-1 inhibits pro-inflammatory cytokine production from activated T cells.

### G6PDi-1 Suppresses Oxidative Burst in Neutrophils

Motivated by the key role of G6PD in effector function in CD4⁺ and CD8⁺ T cells, whether the function of other immune cells depends on G6PD activity was evaluated. In macrophages, G6PDi-1 did not decrease NADPH or LPS-induced pro-inflammatory cytokine production or iNOS upregulation (FIG. 6D). Thus, while in T cells G6PD activity is essential for cytokine production, it is dispensable in the case of LPS-stimulated macrophages (FIG. 6E).

In neutrophils, G6PDi-1 did impact NADPH, albeit to a lesser extent than in T cells. A key function of neutrophils is ROS generation by NADPH oxidase, which requires NADPH and oxygen as substrates. To test the role for the oxPPP in this effector function, mouse and human neutrophils were stimulated with PMA in the presence of 50 µM G6PDi-1 or vehicle control, and oxygen consumption rate was used to readout oxidative burst. G6PDi-1 decreased oxidative burst in both mouse and human neutrophils (FIGS. 6B and 6C). Thus, G6PD activity is essential in providing NADPH for ROS generation by NADPH oxidase in neutrophils.

### Discussion

Small molecule inhibitors with specific on-target activity are key tools for biological research. Unfortunately, however, many tool compounds fail to robustly engage their targets and/or have extensive off-target effects. Here, it is shown that the G6PD inhibitor DHEA, despite clear inhibition of purified enzyme, lacks meaningful on-target cellular activity at doses well above those needed to exhibit anti-proliferative effects. Others have previously raised doubts about DHEA’s cellular G6PD activity, but it has continued to be widely accepted as a G6PD inhibitor, in part because of evidence that it induces oxidative stress. This, however, is a nonspecific outcome, and in the case of DHEA (and several other recently published “G6PD inhibitors”) apparently unrelated to G6PD target engagement.

Substantial chemistry efforts described herein led to a cell-active, on-target G6PD inhibitor, G6PDi-1. G6PDi-1 was then employed to better understand cellular NADPH homeostasis. While the oxPPP is often described as being the canonical, dominant pathway for producing cytosolic NADPH, few studies have directly tested this. As expected, RBCs, which lack mitochondria and therefore the required substrates for producing NADPH when the oxPPP is blocked, were significantly depleted of NADPH upon G6PDi-1 treatment. Many other cell lines were almost completely insensitive. Lymphocytes, however, including primary mouse active CD4+ and CD8+ T cells and human T-ALL cell lines, were yet more sensitive than RBCs. Consistent with this, it was observed that activated T cells do not express substantial ME1 or IDH1, and make NADPH almost solely through the oxPPP, which is strongly upregulated during T cell activation.

If T cells are most sensitive to acute G6PD inhibition, why are clinical manifestations of G6PD deficiency most apparent in RBCs? Activated T cells, unlike mature RBCs, have intense biosynthetic requirements. Previous work has shown that biosynthesis —of proline, deoxyribonucleotides and especially fat — is a major consumer of cytosolic NADPH in proliferating mammalian cells. Additionally, evidence suggests RBCs in G6PD deficient patients are most often impaired through lower levels of enzyme, rather than reduced catalytic function. Unlike T cells, mature RBCs are enucleated, and therefore unable to express new protein. As such, G6PD levels are gradually lost over the life span of RBCs (~120 days), with older RBCs retaining <10% of their original G6PD activity. Mutations in G6PD accelerate this degradation. Indeed, patients possessing G6PD variants with the lowest enzyme stability often experience the worst clinical outcomes. Strikingly, variants that reduce enzyme stability and thereby deplete G6PD activity in RBCs by >95% only modestly impair G6PD activity in leukocytes, often leading to no functional deficit. This makes sense as, in their activated proliferating state, T cells are composed almost solely of freshly made protein. Interestingly, severe G6PD mutations that affect enzyme catalytic ability (rather than protein stability) can present with immune deficiency.

The inability of T cells to maintain NADPH homeostasis upon G6PD blockade did not prevent initial activation or growth, but profoundly inhibited pro-inflammatory cytokine production. Similar cytokine effects were not observed in macrophages, which better maintained NADPH in the face of G6PDi-1. The mechanism linking G6PD to cytokine production remains unclear, but appears to involve defects in transcriptional activation. It is tempting to speculate that previous reports linking restriction of glycolysis-via GLUT1 knockdown, glucose depletion or glucose replacement with 2-deoxyglucose or galactose-with decreased cytokine secretion may be due to oxPPP blockade.

G6PD inhibition resulted in increased total cellular ROS. The general antioxidant N-acetyl-cysteine was able to block the increased ROS but did not restore cytokine secretion. This may reflect the complex role of ROS in immune cell activation, with the right amount required in the right subcellular location. Such a precise ROS control may make T cells uniquely sensitive both to glucose availability and to G6PD inhibition. The in vivo consequences of G6PD inhibition will also reflect its impact on other immune cell types, including suppression of neutrophil oxidative burst, which requires a corresponding burst of NADPH production. G6PDi-1 is a valuable tool for exploring the biological role of G6PD across diverse cellular contexts.

### Materials and Methods

### Cell Lines, Growth Conditions, and Reagents

HCT116, HepG2, L929, LNCap, A549, C2C12, HFF, 293T, Molt4, Jurkat, and SuDHL4 cells were all originally obtained from ATCC (Manassas, VA). 8988T cells were obtained from DSMZ (Braunschweig, Germany). 71-8 cells and iBMK cells were a generous gift from Eileen White (Rutgers Cancer Institute of New Jersey, New Brunswick, NJ). Pooled HUVECs were obtained from ThermoFisher Scientific (#C0155C) and were maintained in EBM-2 Basal Medium (CC-3156, Lonza) supplemented with EGM-2 SingleQuots Supplements (CC-4176, Lonza). All other adherent cell lines (unless otherwise specified) were maintained in DMEM (CellGro 10-017, Mediatech Inc., Manassas, VA) supplemented with 10% fetal bovine serum (F2442, Sigma-Aldrich, St. Louis, MO). All suspension cell lines (unless otherwise specified) were maintained in RPMI-1640 media supplemented with 10% FBS, 100 U/ml penicillin, 100 ug/ml streptomycin and 50 uM 2-mercaptoethanol. All cell lines were routinely screened for mycoplasma contamination. LentiCRISPR v2 (52961) was obtained from Addgene (Cambridge, MA). All primers were synthesized by IDT (Coralville, IA). Antibodies were used according to their manufacturer’s directions. Anti-β-actin (5125) was obtained from Cell Signaling Technologies (Danvers, MA). Anti-G6PD (ab993); ME1 (ab97445) and IDH1 (EPR12296) were obtained from Abcam Inc. (Cambridge, MA). CoxIV antibody was obtained from Proteintech (11242-1-AP.). Standard laboratory chemicals were from Sigma.

### Oligonucleotides

For LentiCRISPR:

| Name | Gene | Targe t exon | PAM sequence | Forward | Reverse |
|---|---|---|---|---|---|
| D19 | non-coding region | -- | ACGGAGGCTAA GCGTCGCAA (SEQ ID NO:1) | CACCGACGGAGG CTAAGCGTCGCA A (SEQ ID NO:2) | CTGCCTCCGAT TCGCAGCGTTC AAA (SEQ ID NO:3) |
| G6pd-1 | G6pd | 5 | CATCTCCTCCCT GTTCCGTG (SEQ ID NO:4) | CACCGCATCTCC TCCCTGTTCCGT G (SEQ ID NO:5) | AAACCACGGA ACAGGGAGGA GATGC (SEQ ID NO:6) |

For RT-PCR:

| | | |
|---|---|---|
| Gene | Forward | Reverse |
| Ifng | TAGCTCTGAGACAATGAACGCTA | GTGATTCAATGACGCTTATGTTG |

| | (SEQ ID NO:7) | (SEQ ID NO:8) |
|---|---|---|
| Gapdh | GCCTTCCGTGTTCCTACCC (SEQ ID NO:9) | CAGTGGGCCCTCAGATGC (SEQ ID NO:10) |
| Rp18s | ACCTGTCTTGATAACTGCCCGTGT (SEQ ID NO:11) | TAATGGCAGTGATGGCGAAGGCTA (SEQ ID NO:12) |

For CRISPR-Cas9 nickase:

| Name | Gene | Target exon | PAM sequence | Forward | Reverse |
|---|---|---|---|---|---|
| mPgd-1 | Pgd | 3 | TCATGTTCAG AATTAAGTTC (SEQ ID NO:15) | CACCGTCATG TTCAGAATTA AGTTC (SEQ ID NO:16) | AAACGAACTT AATTCTGAAC ATGAC (SEQ ID NO:17) |
| mPgd-2 | Pgd | 3 | CGGCTTTGTG GTAAGCGGC G (SEQ ID NO:18) | CACCGCGGCT TTGTGGTAAG CGGCG (SEQ ID NO:19) | AAACCGCCGC TTACCACAAA GCCGC (SEQ ID NO:20) |

### G6PD Plasmid Construction and Expression

Partially truncated human G6PD (residues 28-515, Uniprot ID P11413) was subcloned into the pET28a vector using the Ndel and Xhol restriction enzyme sites and the following primers: 5′-agtcagcatatggtcagtcggatacacacatattcatc-3′ (SEQ ID NO:13) and 5′-agtcagctcgagtcagagcttgtgggggttcac-3′ (SEQ ID NO:14). Recombinant G6PD was expressed in Escherichia coli BL21(De3)pLysS as an N-terminal His₆-tagged protein with an integrated thrombin cleavage site. Briefly, IPTG was added (final concentration of 1 mM) to induce protein expression when culture density reached an OD₆₀₀ of 0.6, followed by incubation at 37° C. overnight. Pellets were isolated and lysed by sonication in buffer containing 50 mM Tris (pH 8), 500 mM NaCl, 20 mM imidazole, 1 mM BME, 1 mM PMSF, and 5% glycerol v/v. The lysate was centrifuged and filtered to remove insoluble debris. The resulting supernatant was fractionated twice with ammonium sulfate; first to 25% at 4° C. for 1 hour, with the supernatant undergoing subsequent fractionation to 50% at 4° C. for 1 hour. The precipitate was collected and dissolved in binding buffer consisting of 50 mM NaH₂PO₄, Tris (pH 8), 500 mM NaCl, 20 mM imidazole, and 1 mM BME, and was loaded onto a Ni Sepharose HisTrap HP column (GE Healthcare, 17-5248-01). The column was washed with approximately 10 column volumes of binding buffer. Elution of G6PD was achieved with elution buffer consisting of 50 mM NaH₂PO₄, Tris (pH 8), 500 mM NaCl, 250 mM imidazole, and 1 mM BME. The eluted protein was desalted and concentrated to remove the imidazole before undergoing thrombin cleavage using a Thrombin CleanCleave Kit (MilliporeSigma, C974M34). The tag-less protein was purified by size-exclusion chromatography using a Superdex 200 Increase 10/300 GL column (GE Healthcare) using buffer consisting of 50 mM Tris (pH 8), 150 mM NaCl, and 1 mM BME. Eluted protein was concentrated using an Amicon Ultra 10 kDa MWCO filter (MilliporeSigma, UFC901008). Protein concentration was determined by Pierce BCA Assay (Thermo, 23225) and was stored in 10% glycerol at -80° C.

### G6PD Enzymatic Activity Measurement by Diaphorase-Coupled Assay

Inhibitor activity against recombinant human G6PD was determined using a resazurin-based diaphorase coupled kinetic assay. Increasing doses of test compound in 96-well, opaque-bottomed plates were treated with assay buffer containing 50 mM triethanolamine pH = 7.4, 1 mM MgCl₂, 0.1 mM resazurin, 0.03 mM NADP+, 0.1 U/mL Clostridium kluyveri diaphorase (MilliporeSigma, D5540), 0.25 mg mL⁻¹ bovine serum albumin, and approximately 1 nM purified G6PD. The reaction was initiated by the addition of 1 mM glucose-6-phosphate (G6P). Plates were incubated at 30° C. and read every minute by a BioTek plate reader (Synergy HT) monitoring fluorescence emission at 560 nm following excitation at 530 nm. For Michaelis-Menten experiments NADP+ and G6P concentrations were varied as described. For jump dilution experiments, inhibitor was initially incubated in assay buffer containing approximately 10 nM G6PD for 30 minutes at 30° C. (1x dilution), before being diluted (1:50) into an equal volume of assay buffer containing no G6PD or inhibitor (50x dilution), followed by reaction initiation of both by the addition of 1 mM G6P. G6PD inhibition was determined by calculating the change in relative fluorescence over time (RFU/min) in the presence of different doses of test compound, followed by normalization against control wells without compound. GraphPad Prism (v7.1) was used to perform a non-linear curve fit (4-parameter) to determine IC₅₀ values.

### Generation of G6PD-null Cell Lines

Generation of clonal G6PD-null lines in the HCT116 background have been previously described. Generation of batch G6PD-null cells in the HepG2 background was achieved using the lentiviral CRISPR-Cas9 vector system LentiCRISPR v2 (Addgene #52961). Briefly, sgRNA sequences targeting exon-5 of human G6pd were designed using the crispr.mit.edu design tool. The identified PAM sequences (see table above) were subcloned into the LentiCRISPR v2 using the BsmBI restriction endonuclease (NEB R0580S). Virus was produced through PEI (MilliporeSigma, 408727) transfection of vectors and lentiviral packaging plasmids psPax2 and VSVG in 293T cells. Medium containing lentivirus was collected after two days and filtered through a PES filter (0.22 um, MilliporeSigma). HepG2 cells were transfected with virus targeting non-coding control or G6pd and Polybrene (8 ug/mL, Invitrogen). Cells were split after 48 hours into RPMI-media (10% FBS) containing puromycin (2 ug/mL) and cultured for 3 days. G6PD knockout was confirmed by Western blotting.

### G6PD Enzymatic Activity Measurement by LC-MS

Inhibitor activity against recombinant human G6PD was determined by direct product monitoring by LC-MS. Test compounds at indicated doses were treated with assay buffer containing 50 mM triethanolamine pH = 7.4, 1 mM MgCl₂, 0.30 mM NADP+, 0.25 mg/mL bovine serum albumin, and approximately 1 nM purified G6PD. The reaction was initiated by the addition of 1 mM G6P (or water for negative control condition). Aliquots of reaction mixture were collected at indicated time points and rapidly quenched by diluting (1:5) into methanol precooled on dry ice. The mixtures were centrifuged at 13,000 rcf for 20 minutes at 4° C., and the resulting supernatants were diluted (1:20) into 40:40:20 methanol/ acetonitrile/ water and analyzed by LC-MS.

### Chemical Synthesis

### Synthesis of Compound 2 (G6PDi-1):

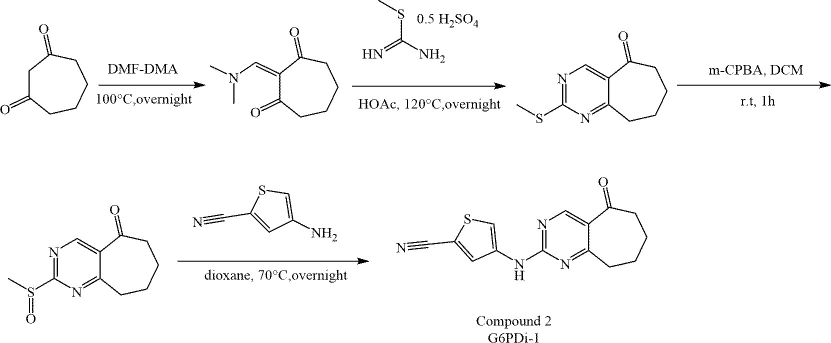

### Synthesis of 2-[(dimethylamino)methylidene]cycloheptane-1,3-dione:

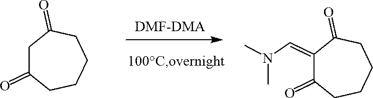

Into a 100-mL, round-bottomed flask was placed cycloheptane-1,3-dione (5 g, 39.634 mmol, 1 equiv), (dimethoxymethyl)dimethylamine (DMF-DMA; 10 mL). The resulting solution was stirred overnight at 100° C. The reaction mixture was cooled to room temperature and concentrated under vacuum. This resulted in 7.0 g (crude) of 2-[(dimethylamino)methylidene]cycloheptane-1,3-dione as a brown solid. LCMS: (ES, m/z): 182 [M+H]⁺.

### Synthesis of 2-(methylsulfanyl)-5H, 6H, 7H, 8H, 9H-cyclohepta[d]pyrimidin-5-one:

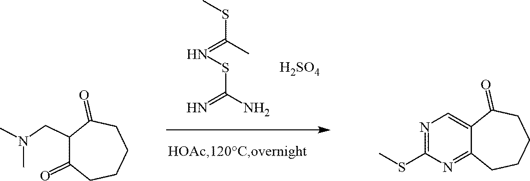

Into a 50-mL, round-bottomed flask was placed 2-[(dimethylamino)methylidene]cycloheptane-1,3-dione (5.1 g, 28.140 mmol, 1 equiv), bis((methylsulfanyl)methanimidamide) sulfuric acid salt (3.92 g, 14.070 mmol, 0.5 equiv), and glacial acetic acid (HOAc; 10 mL). The resulting solution was stirred overnight at 120° C. The reaction mixture was cooled to room temperature and poured into 10 g of ice water. The pH of the solution was adjusted to 6 with Na₂CO₃ (solid). The resulting solution was extracted with 3×30 mL of ethyl acetate and the organic layers combined and dried over anhydrous sodium sulfate, filtered and concentrated. The residue was applied onto a silica gel column and eluted with ethyl acetate/petroleum ether (1:5). This resulted in 4 g (68.25%) of 2-(methylsulfanyl)-5H, 6H, 7H, 8H, 9H-cyclohepta[d]pyrimidin-5-one as a yellow solid. H-NMR: ¹H NMR (300 MHz, DMSO-d6) δ 8.69 (s, 1H), 3.09 - 3.05 (m, 2H), 2.80 - 2.75 (m, 2H), 2.33(s, 3H), 1.92 - 1.78 (m, 4H).

### Synthesis of 2-methanesulfinyl-6H, 7H, 8H, 9H-cyclohepta[d]pyrimidin-5-one:

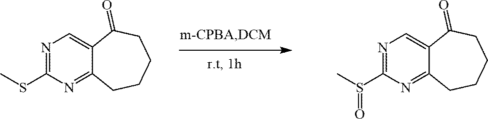

Into a 50-mL, round-bottomed flask was placed 2-(methylsulfanyl)-6H,7H,8H,9H-cyclohepta[d]pyrimidin-5-one (208.00 mg, 0.999 mmol, 1.00 equiv), dichloromethane (DCM; 3.00 mL), and meta-chloroperbenzoic acid (m-CPBA; 206.00 mg, 0.999 mmol, 1.0 equiv, 85% purity). The resulting solution was stirred for 1 hour at room temperature. The reaction was then quenched by the addition of 5 mL of water. The resulting solution was extracted with 3×5 mL of dichloromethane, and the organic layers combined and dried over anhydrous sodium sulfate, filtered and concentrated under vacuum. This resulted in 200 mg (crude) of 2-methanesulfinyl-6H, 7H, 8H, 9H-cyclohepta[d]pyrimidin-5-one as a white solid. LC-MS: (ES, m/z): 225 [M+H]⁺.

### Synthesis of 4-([5-oxo-6H,7H,8H,9H-cyclohepta[d]pyrimidin-2-yl]amino)thiophene-2-carbonitrile (Compound 2, G6PDi-1):

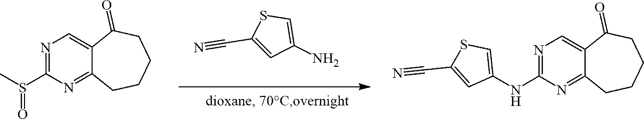

Into a 8-mL vial was placed 2-methanesulfinyl-6H, 7H, 8H, 9H-cyclohepta[d]pyrimidin-5-one (100.00 mg, 0.446 mmol, 1.00 equiv), dioxane (3.00 mL), and 4-aminothiophene-2-carbonitrile (55.36 mg, 0.446 mmol, 1.00 equiv). The resulting solution was stirred overnight at 70° C. The reaction mixture was cooled to room temperature and concentrated under vacuum. The residue was applied onto a silica gel column and eluted with ethyl acetate/petroleum ether (1:1). The collected fractions were combined and concentrated. The crude product was purified by Prep-HPLC with the following conditions ((Prep-HPLC-006): Column, XBridge Shield RP18 OBD; mobile phase, Water (10 mMOL/L NH₄HCO₃+0.1%NH₃.H₂O) and acetonitrile (ACN; 35% ACN up to 55% in 7 minutes); Detector, 220 nm. This solution was dried by lyophilization. This resulted in 40 mg (31.52%) of 4-([5-oxo-6H,7H,8H,9H-cyclohepta[d]pyrimidin-2-yl]amino)thiophene-2-carbonitrile as an off-white solid. LC-MS: (ES, m/z): 285 [M+H] ⁺. H-NMR: ¹H NMR (300 MHz, DMSO-d6) δ 8.70 (s, 1H), 8.16 (brs, 1H), 7.97 (d, J = 1.5 Hz, 1H), 3.06 (m, 2H), 2.77 - 2.73 (m, 2H), 1.86 - 1.79 (m, 4H).

### Synthesis Of Compound “Neg-Ctrl”:

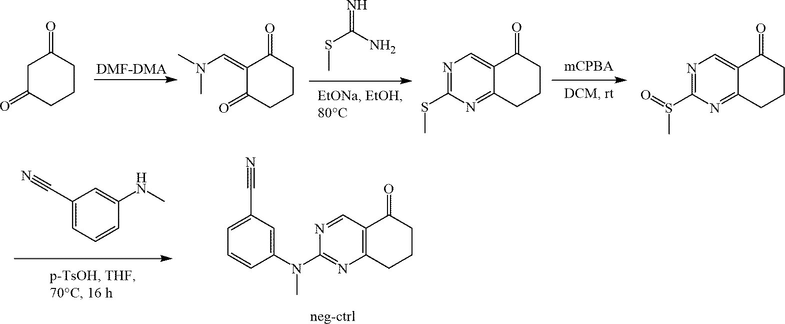

### Synthesis of 2-((dimethylamino)methylene)cyclohexane-1,3-dione:

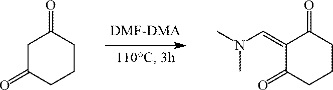

Into a 100-mL, round-bottomed flask was placed cyclohexane-1,3-dione (9.2 g, 80 mmol) in DMF-DMA (20 mL). The resulting solution was stirred for 3 hours at 110° C. The reaction mixture was cooled to room temperature and concentrated under vacuum. This resulted in 15 g (crude) of 2-((dimethylamino)methylene)cyclohexane-1,3-dione as a brown solid.

### Synthesis of 2-(methylsulfanyl)-5,6,7,8-tetrahydroquinazolin-5-one:

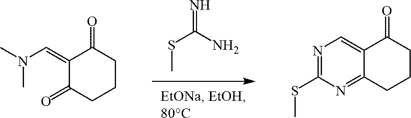

Into a 100-mL, round-bottomed flask was placed 2-[(dimethylamino)methylidene]cyclohexane-1,3-dione (3 g, 17.94 mmol), bis((methylsulfanyl) methanimidamide) sulfuric acid (2.5 g, 8.98 mmol), sodium ethoxide (EtONa; 3.7 g), and ethanol (EtOH; 30 mL). The resulting solution was stirred for 2 hours at 80° C. The reaction mixture was cooled to room temperature and concentrated under vacuum. The residue was applied onto a silica gel column and eluted with ethyl acetate. This resulted in 510 mg (15%) of 2-(methylsulfanyl)-5,6,7,8-tetrahydroquinazolin-5-one as a yellow solid. Synthesis of 2-methanesulfinyl-5,6,7,8-tetrahydroquinazolin-5-one:

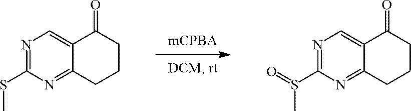

Into a 50-mL, round-bottomed flask was placed 2-(methylsulfanyl)-5,6,7,8-tetrahydroquinazolin-5-one (200 mg, 1.03 mmol) and dichloromethane (2 mL). m-CPBA (195 mg, 1.13 mmol) was added. The resulting solution was stirred for 1 hour at room temperature. The resulting mixture was concentrated under vacuum. This resulted in 210 mg (crude) of 2-methanesulfinyl-5,6,7,8-tetrahydroquinazolin-5-one as a yellow solid. Synthesis of 3-[methyl(5-oxo-5,6,7,8-tetrahydroquinazolin-2-yl)amino]benzonitrile (neg-ctrl):

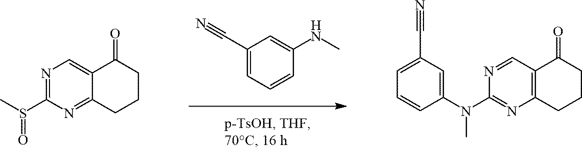

Into a 8-mL vial was placed 2-methanesulfinyl-5,6,7,8-tetrahydroquinazolin-5-one (159 mg, 0.75 mmol), 3-(methylamino)benzonitrile (100 mg, 0.75 mmol), tetrahydrofuran (THF; 2 mL), and p-TsOH (13 mg, 0.075 mmol). The resulting solution was stirred for 16 hours at 70° C. The resulting mixture was cooled to room temperature and concentrated under vacuum. The crude product was purified by Prep-HPLC with the following conditions: Column, XBridge Prep C18 OBD 19*150 mm*5 um; mobile phase, A: water (10 mmol/L NH₄HCO₃); B: CN; 29-48%B in 6 minutes; flow rate: 20 ml/min; Detector, 220 nm. This resulted in 0.03 g (14%) of 3-[methyl(5-oxo-5,6,7,8-tetrahydroquinazolin-2-yl)amino]benzonitrile as a yellow solid. LC-MS: (ES, m/z): 279.1 [M+H]⁺. H-NMR: (300 MHz, DMSO, ppm): δ 8.71 (s, 1H), 7.94 (s, 1H), 7.78-7.72 (m, 2H), 7.65-7.60 (m, 1H), 3.56 (s, 3H), 2.85-2.81 (t, J= 6.0 Hz, 2H), 2.56-2.51 (m, 2H), 2.08-1.98 (m, 2H).

### Mice

Animal studies followed protocols approved by the Princeton University Institutional Animal Care and Use Committee (protocol number 2032-17). Seven- to ten-week-old mice were used for all experiments. Wild-type C57BL/6 were purchased from Charles River. The mice were on normal light cycle (8 AM - 8 PM) and had free access to water and standard chow diet.

### Isolation, Culture and Stimulation of Naïve CD8⁺ or CD4⁺ T cells

To isolate naïve CD8⁺ or CD4⁺ T cells, spleens were harvested and single cell suspensions prepared by manual disruption and passage through a 70-µm cell strainer in PBS supplemented with 0.5% BSA and 2 mM EDTA. After red blood cell lysis, naïve CD8⁺ or CD4⁺ T cells were purified by magnetic bead separation using commercially available kits following vendor instructions (Naive CD8a+ T Cell Isolation Kit, mouse or Naive CD4+ T Cell Isolation Kit, mouse, Miltenyi Biotec Inc).

Cells were cultured in complete RPMI media (RPMI 1640 supplemented with 10% FBS, 100 U/ml penicillin, 100 µg/ml streptomycin, and 50 µM 2-mercaptoethanol). Naïve T cells were either rested in complete RPMI media supplemented with recombinant IL-7 (50U/mL) or stimulated for 48 hours with plate-bound anti-CD3 (10 µg/ml) and anti-CD28 (5 µg/ml) in complete RPMI media supplemented with 100 µM alanine and recombinant IL-2 (100 U/mL). Activated T cells were maintained in complete RPMI media supplemented with recombinant IL-2 (100 U/mL). All experiments on “active” T cells were performed at day 4-5 post-activation

### Isolation, Culture and Stimulation of Bone Marrow-Derived Macrophages

Mouse bone marrow monocyte/macrophage progenitors were isolated from femur and tibia and cultured in BMM media (DMEM supplemented with 10% FBS, 20% L929-conditioned media, 100 U/ml penicillin, and 100 µg/ml streptomycin). Expression of CD11b and F4/80 was checked by flow cytometry after 6 days in culture. Mature macrophages were either maintained in BMM media (M0 macrophages) or stimulated overnight with LPS (100 ng/mL) + IFNγ (20 ng/mL) for M1 activation or IL-4 (20 ng/mL) for M2 activation.

### Isolation and Culture of Primary Mouse Hepatocytes

Primary hepatocytes were isolated from C57B1/6 mice by perfusion of the liver with liver perfusion medium (1x) (Thermo Fisher 17701038) followed by digestion with one bottle of collagenase/elastase (Worthington Biochemical LK002066) and DNase1 (Worthington Biochemical, LK003170) in Krebs Ringer Buffer with HEPES and 0.5 mM CaCl₂. Digested liver was minced in hepatocyte wash medium (Thermo Fisher 17704024), passed through a 70-µm strainer, and centrifuged at 50 g. Dead cells were removed by adding a 25% percoll solution, centrifuging at 120 g, and aspirating the supernatant. Primary hepatocytes were plated at 1.2 M cells/well in collagen-coated, 6-well plates in pre-warmed DMEM with 100 nM insulin, 100 nM dexamethasone, and 1% Glutamax.

### Isolation and Culture of Erythrocytes From Mouse Spleen

To isolate red blood cells, mice were euthanized by cervical dislocation followed by collection of approximately 200 µL whole blood via cardiac puncture into tubes containing 7.5 µL heparin (1000USP/mL, H3393, Sigma Aldrich). The cells were incubated on ice for approximately 5 minutes, then centrifuged (5 minutes, 500 rpm, 4° C.) followed by aspiration of the serum and buffy coat layer. Cells were gently resuspended in PBS and then pelleted (5 minutes, 500 rpm, 4° C.) three times. Cells were then resuspended in RPMI media and used immediately for experiments.

### Flow Cytometry Analysis

To analyze cell surface markers expression, cells were collected, washed with PBS and stained with the viability dye Live/Dead Aqua. Cells were then washed with staining buffer and stained for surface markers: CD4 (APC-Cy7, clone RM4-5,), CD8a (PerCP-Cy5.5, clone 53-6.7), CD25 (APC, clone PC61), CD44 (PE-Cy7, clone IM7), CD62L (PE, clone MEL-14) CD69 (FITC, clone H1.2F3), for T cells; CD11b (APC, clone M1/70 and F4/80 (FITC, clone BM8) for macrophages.

To analyze proliferation, naïve CD8 T cells were stained with CTV dye and either maintained in a naïve state with IL7 or stimulated with αCD3/αCD28 + recombinant IL-2 in the presence of increasing concentrations of G6PDi-1. Cells were refed at days 2 and 3 post-stimulation, and proliferation was measured at day 4 post-activation. Cells were collected, washed with staining buffer and stained with the viability dye propidium iodide.

To analyze intracellular cytokines, active T cells were re-stimulated with PMA (20 ng/ml) and ionomycin (1 µg/ml) in the presence of GolgiStop and increasing concentrations of G6PDi-1. After a 6-hour incubation period, cells were collected, washed with PBS and stained with the viability dye Live/Dead Aqua. Cells were then washed with staining buffer and stained for surface markers: CD4 (APC-Cy7, clone RM4-5,), CD8a (PerCP-Cy5.5, clone 53-6.7). Cells were then fixed and permeabilized and stained for intracellular cytokines: IFN-γ (FITC, clone XMG1.2), TNFα (PE-Cyanine7, clone MP6-XT22) and IL-2 (PE, clone JES6-5H4). To analyze intracellular cytokines, mature macrophages were stimulated with LPS (100 ng/mL) + IFNγ (20 ng/mL) in the presence of GolgiStop and increasing concentrations of G6PDi-1. After a 6-hour incubation period, cells were collected, washed with PBS and stained with the viability dye Live/Dead Aqua. Cells were then fixed and permeabilized and stained for intracellular cytokines: TNFα (PE-Cyanine7, clone MP6-XT22) and IL-10 (V450, clone JES5-16E3).

For measuring intracellular ROS levels, cells were incubated for 2 hours in the presence of increasing concentrations of G6PDi-1 and then stained for ROS and viability using CellROX Green Flow Cytometry Assay Kit following manufacturer instructions. In some experiments, n-acetyl cysteine (1 mM), galactose oxidase (0.045 U/mL) + galactose (500 µM), or potassium superoxide (0.5 µg/mL) were added to the culture media at the same time the drug and/or PMA and ionomycin were added. All flow cytometry was analyzed with an LSR II (BDbiosciences) using standard filter sets and FCS express 6 flow cytometry software (De Novo Software).

### Absolute Quantification of oxPPP Flux Using 1-¹⁴C-glucose and 6-¹⁴C-glucose

Glucose oxidation flux through oxPPP was determined from difference in the rate of ¹⁴CO₂ released from [1-¹⁴C]-glucose and [6-¹⁴C]-glucose, as previously described with some modification. RPMI 1640 media without sodium bicarbonate was supplemented with 0.74 g/L of NaHCO₃, 2.5 mM HEPES pH 7.4, 10% dFBS, and 1% of either 1-¹⁴C-glucose or 6-¹⁴C-glucose. Three million primary mouse naïve (rested in IL-7) or active CD8+ T cells (in the presence of IL2 and 0.1% DMSO or 10 uM G6PDi-1) were incubated for 4 hours in a sealed 12.5 cm² flask. To facilitate the collection of ¹⁴CO₂, 100 µL of a 10 M KOH solution was introduced into the sealed flask using a center well for incubation flask (8823200000, DWK Life Sciences). The assay was stopped by injection of 1 mL 1 M HCL, and the KOH solution then transferred to scintillation vials containing 10 mL scintillation solution for counting. The signal was corrected for the percentage of radioactive tracer in the medium. OxPPP flux is calculated as follows:

$$\begin{matrix}\text{oxPPP flux} \left(\text{fmol} {\text{h}}^{\text{-1}} {\text{cell}}^{\text{-1}}\right) = \frac{{}^{\text{14}} {\text{CO}}_{\text{2}} \left(\text{nmol}\right)}{\text{Cell number} \left(\text{millions}\right) \times \text{labeling time} \left(\text{h}\right)} \times \\ \frac{\text{Total glucose} \left(\text{nmol}\right)}{{}^{14} \text{C} \text{-} \text{glucose} \left(\text{nmol}\right)}\end{matrix}$$

### Tracer Experiments

For all experiments involving stable isotope tracers (e.g., 1-²H-glucose), the isotope tracer nutrient was substituted for unlabeled nutrient at the same concentration normally found in the base media for a given cell type (e.g., DMEM for HCT116, RPMI for T cells, etc.). In addition, dialyzed FBS was used as a supplement in place of FBS.

Cytosolic NADPH sources were traced, and redox active hydride labeling was calculated, using a previously described strategy. 1-²H-glucose (which directly traces G6PD) or 3-²H-glucose (which directly traces PGD) were used for tracing oxPPP contribution to NADPH; 4-²H-glucose for ME1; [2,3,3,4,4-²H₅]-glutamine, for both ME1 and IDH1; and D₂O, to account for solvent exchange.

The mass difference between ¹³C₁ and ²H₁ NADPH and NADP⁺ cannot be resolved using the Q Exactive Plus. Therefore, the natural ¹³C abundance was corrected from the raw data. The labeling of the redox-active hydrogen of NADPH ([Active-H]) and correction for solvent exchange were done as previously described. 1-²H-glucose and 3-²H-glucose contribution were corrected by glucose-6-phosphate labeling, 4-²H-glucose by malate labeling and [2,3,3,4,4-²H₅]-glutamine by the average of citrate and malate labeling. OxPPP contribution was calculated as the sum of the normalized active H labeling for 1-²H-glucose and 3-²H-glucose, and ME1 plus IDH1 as the sum of the normalized active H labeling for 4-²H-glucose and [2,3,3,4,4-²H₅]-glutamine.

### Metabolite Extraction

For analysis of intracellular metabolites by LC-MS, adherent cell lines were plated and grown to 80% confluency in 6-well plates. At the start of an experiment, the appropriate media was added to cells, which included isotope tracers and/or chemical inhibitors as described. Cells were incubated at 37° C. at 5% CO₂ for 2 hours (unless otherwise noted). For all experiments involving small molecule agents, DMSO concentrations were <0.2%. After 2 hours, media was removed by aspiration and metabolome extraction was performed (without any wash steps) by the addition of 800 µL of ice cold solvent (40:40:20 acetonitrile:methanol:water + 0.5% formic acid). After a 1-minute incubation on ice, the extract was neutralized by the addition of NH₄HCO₃ (15% w/v). The samples were incubated at -20° C. for approximately 30 minutes, at which point the wells were scraped and the extract transferred to Eppendorf tubes and centrifuged (15 minutes, 16000 rpm, 4° C.). The resulting supernatant was frozen on dry ice and kept at -80° C. until LC-MS analysis.

For suspension cells (including T cells and red blood cells), 2×10⁶ cells were seeded in 1 mL of media in 12-well plates and incubated with appropriate media, which included isotope tracers and/or chemical inhibitors and/or cytokines, as described. For all experiments involving small molecule agents, DMSO concentrations were <0.2%. After 2 hours, cell suspensions were transferred to 1.5 mL tubes and pelleted (30 seconds, 6000 rpm, room temperature). Media was removed by aspiration and metabolome extraction was performed by the addition of 75 µL of ice cold solvent (40:40:20 ACN:MeOH:H₂O + 0.5%FA). After a 5-minute incubation on ice, acid was neutralized by the addition of NH₄HCO₃. After centrifugation (15 minutes, 16000 rpm, 4° C.), the resulting supernatant was transferred to a clean tube, frozen on dry ice and kept at -80° C. until LC-MS analysis.

### LC-MS Analysis

Metabolites were analyzed using a quadrupole-orbitrap mass spectrometer (Q Exactive Plus, Thermo Fisher Scientific, Waltham, MA), coupled to hydrophilic interaction chromatography (HILIC) with LC separation on a XBridge BEH Amide column (Waters), or a stand-alone orbitrap (Thermo-Fisher Exactive) coupled to reversed-phase ion-pairing chromatography with LC separation on a HSS-T3 column (Waters). Both mass spectrometers were operating in negative ion mode and were coupled to their respective liquid chromatography methods via electrospray-ionization. Detailed analytical conditions have been previously described.

Adherent cell metabolite abundances were normalized by packed cell volume; suspension cells to cell count. Unless otherwise indicated, isotopic labeling of metabolites arising from incubation with ¹³C or ²H labeled nutrients were corrected for natural abundance, as previously described. Data were analyzed using the ElMaven software (v 0.2.4, Elucidata), with compounds identified based on exact mass and retention time match to commercial standards. For metabolomics analysis, metabolites data were normalized to control condition and clustered using Cluster 3.0 software. Heatmaps were plotted using Java Treeview.

### Absolute Quantification of NADP+ and NADPH in Active CD8+ T Cells

Active CD8+ T cells were cultured and metabolome extraction was performed as previously described. Packed cell volume was measured using Midwest Scientific PCV cell counting tubes and estimated to be 1.5 uL per 2×10⁶ cells. Cell extracts were spiked with 1.5 µL of NADP⁺ 2.5 or 25 µM or NADPH 20 or 200 µM. Absolute concentration was calculated based on the increase in NADP+ or NADPH signal in the spiked samples.

### Statistics

Sample sizes are defined in each figure legend. Results for technical replicates are presented as mean ± SD. Statistical significance between conditions was calculated using unpaired Student’s t-test (two-tailed) when comparing two groups, and one-way ANOVA followed by Dunnett’s post hoc analysis when comparing three. All statistical calculations were performed using the software package GraphPad Prism 7.03.

### Generation of G6PD-null and Hypomorphic PGD Cell Lines

Generation of clonal G6PD-null line in the HCT116 background has been previously described. Using a similar approach, a clonal hypomorphic PGD cell line in the HCT116 background was generated. Briefly, paired guide RNAs (mPgd-1 and mPgd-2) against exon 3 of human Pgd (see above oligonucleotide table) were cloned into plasmids containing Cas9 nickase expression vector and puromycin-resistant genes. Cells were transiently transfected with these plasmids using Lipofectamine 3000 (Life Technologies) and selected for 48 hours with 2 µg/mL puromycin. After selection, cells were grown to confluence before single-cell plating in 96-well plates. Functional gene deletion was confirmed by targeted genomic sequencing followed by Western blotting.

Generation of batch G6PD-null cells in the HepG2 background was achieved using the lentiviral CRISPR-Cas9 vector system LentiCRISPR v2 (Addgene No. 52961). Briefly, sgRNA sequences targeting exon-5 of human G6pd were designed using the crispr.mit.edu design tool. The identified PAM sequences (see oligonucleotide table above) were subcloned into the LentiCRISPR v2 using the BsmBI restriction endonuclease (NEB R0580S). Virus was produced through PEI (MilliporeSigma, 408727) transfection of vectors and lentiviral packaging plasmids psPax2 and VSVG in 293T cells. Medium containing lentivirus was collected after two days and filtered through a PES filter (0.22 µm, MilliporeSigma). HepG2 cells were transfected with virus targeting non-coding control or G6pd and Polybrene (8 ug/mL, Invitrogen). Cells were split after 48 hours into RPMI media (10% FBS) containing puromycin (2 ug/mL) and cultured for 3 days. G6PD knockout was confirmed by Western blotting.

FIG. 5A is a schematic depicting direct monitoring of 6-pg by LC-MS in HCT116-mPgd cells. FIG. 5B is an image of a Western blot comparing G6PD and PGD expression in clonal mPgd line, generated using CRISPR-Cas9. FIG. 5C shows 6-pg total ion counts in HCT116, G6pdΔ, and mPgd lines (mean ± SD, n = 4, one-way ANOVA). Hypomorphic PGD activity leads to elevated 6-pg levels. FIG. 6A shows 6-pg dose-response curves in 6-phophogluconate hypomorphic HCT116 cells (HCT116-mPgd cells) (mean ± SD, n = 3).

### Isolation, Culture, and Stimulation of Neutrophils

Murine neutrophils were isolated from 8-12-week old C56BL/6 mice. Mice were bred and maintained according to the University of Wisconsin Institutional Animal Care and Use Committee (Protocol No. M006219). Mice were euthanized by cervical dislocation, and bone marrow cells were harvested from femur and tibia within 30 minutes. Cell suspensions were passed through a 70-µm cell strainer. Neutrophils were prepared using a negative selection kit (EasySep Mouse Neutrophil Enrichment Kit, Stem Cell Technologies), following the manufacturer’s instructions. Cells were cultured in RPMI 1640 media supplemented with 10% heat-inactivated FBS, 100 U/ml penicillin, 100 µg/ml streptomycin, 5 mM HEPES, and 2 mM EDTA. To stimulate neutrophils, 100 nM phorbol 12-myristate 13-acetate (PMA) (Cayman Chemical) was added to the media; metabolites were extracted after 30 minutes of stimulation.

Human neutrophils were isolated from 8 ml of blood collected from healthy donors following the protocol approved by the University of Wisconsin Institutional Review Board (Protocol No. 2019-1031-CP001). Neutrophils were purified using the MACSxpress Whole Blood Neutrophil Isolation Kit (Miltenyi Biotec 130-104-434) followed by erythrocyte depletion (Miltenyi Biotec 130-098-196) according to the manufacturer’s instructions.

### Neutrophil Oxidative Burst Assay

Suspended neutrophils were plated in culture wells pre-coated with CELL-TAK™ (Corning), spun at 200 × g for 1 minute with minimal acceleration/deceleration, and then incubated for 1 hour at 37° C. Murine neutrophils were plated at 2×10⁵ cells/well in RPMI 1640 media without sodium bicarbonate. Human neutrophils were plated at 5×10⁴ cells/well in RPMI 1640 media supplemented with 0.1% human serum albumin. Inhibitor (G6PDi-1, 50 µM) or vehicle control were added just prior to starting the assay. Mouse neutrophils were also treated with rotenone (0.5 µM, BioVision) + antimycin A (0.5 µM, BioVision) at t = 20 minutes. Oxidative burst was stimulated with PMA (100 nM, Cayman Chemical); oxygen consumption rate was measured using the XF-96e extracellular flux analyzer (Seahorse Bioscience).

FIGS. 6B and 6C show that G6PDi-1 suppresses oxidative burst in neutrophils.

### Cellular Thermal Shift Assay (CETSA)

Lysates from HepG2 cells at 75% confluence were isolated with 0.5% Triton in TBS (20 mM, Tris pH 7.4, 150 mM NaCl) for 30 min on ice, pre-cleared by centrifugation and used for thermal shift assay as described 49. Briefly, inhibitor or DMSO control was added to lysates at indicated concentration and incubated for 30 min on ice followed by 3 min heating at 47° C., 50° C., 53° C., and 56° C. in a thermal cycler. After heating, tubes were cooled at room temperature for 3 min and insoluble fraction removed by centrifugation at 17,000 g for 20 min. The soluble fraction was separated by SDS-PAGE, transferred to PVDF membrane, and immunoblotted using indicated antibodies at a dilution of 1:2000. Blots were developed by chemiluminescence and imaged using LI-COR C-DiGit Western Blot Scanner. Two independent experiments were performed. Signal intensity of proteins from immunoblots was quantified using Image Studio version 5.2 for C-DiGit Scanner, and bands were normalized to signal intensity of the 47° C. treated samples. Relative signal intensities were plotted as bar graph relative to the DMSO treated control.

### Treg Assays

Spleen and peripheral lymph nodes were harvested and processed to single cell suspensions of lymphocytes. Red blood cells were removed with hypotonic lysis. We used magnetic beads (Miltenyi Biotec, San Diego, CA) for isolation of Tconv (CD4⁺CD25⁻), Treg (CD4⁺CD25⁺), and antigen presenting cells (CD90.2⁻). For cell culture medium, we used RPMI 1640 medium supplemented with 10% fetal bovine serum (FBS), penicillin (100 U/mL), streptomycin (100 µg/mL), and 55 µM β-mercaptoethanol Treg suppression and iTreg polarization were conducted as previously reported. For Treg suppression assays, Tconv were purified and stimulated with irradiated antigen presenting cells plus CD3ε mAb (1 µg/mL, BD Pharmingen). To assess proliferation, Tconv cells were labeled with carboxyfluorescein succinimidyl ester (CFSE), and Treg cells with CellTrace Violet. After 72 h, proliferation of Tconv and Treg cells was determined by flow cytometric analysis of CFSE and CellTrace Violet dilution, respectively. For conversion to Foxp3⁺ Tregs, Tconv cells were incubated for 3-5 days with CD3ε/CD28 mAb beads, plus TGF-β (3 ng/mL) and IL-2 (25 U/mL), and analyzed by flow cytometry for Foxp3+ iTreg. Flow cytometry data was captured using Cytoflex (Beckman Coulter, Brea, CA) and analyzed using the FlowJo 10.2 software.

### REFERENCES

1 Voet, D., Voet, J. G. & Pratt, C. W. Fundamentals of biochemistry: life at the molecular level (2016).

2 Stanton, R. C. Glucose-6-Phosphate Dehydrogenase, NADPH, and Cell Survival. IUBMB life 64, 362-369, doi:10.1002/iub.1017 (2012).

3 Uhlen, M. et al. Proteomics. Tissue-based map of the human proteome. Science (New York, N.Y.) 347, 1260419, doi:10.1126/science.1260419 (2015).

4 Kowalik, M. A., Columbano, A. & Perra, A. Emerging Role of the Pentose Phosphate Pathway in Hepatocellular Carcinoma. Frontiers in Oncology 7, doi:10.3389/fonc.2017.00087 (2017).

5 Zhang, Q. et al. Overexpression of G6PD Represents a Potential Prognostic Factor in Clear Cell Renal Cell Carcinoma. Journal of Cancer 8, 665-673, doi:10.7150/jca.16858 (2017).

6 Nagashio, R. et al. Prognostic significance of G6PD expression and localization in lung adenocarcinoma. Biochimica et Biophysica Acta (BBA) - Proteins and Proteomics 1867, 38-46, doi:https://doi.org/10.1016/j.bbapap.2018.05.005 (2019).

7 Pu, H. et al. Overexpression of G6PD is associated with high risks of recurrent metastasis and poor progression-free survival in primary breast carcinoma. World journal of surgical oncology 13, 323-323, doi:10.1186/s12957-015-0733-0 (2015).

8 Longo, L. et al. Maternally transmitted severe glucose 6-phosphate dehydrogenase deficiency is an embryonic lethal. The EMBO journal 21, 4229-4239, doi:10.1093/emboj/cdf426 (2002).

9 Cappellini, M. D. & Fiorelli, G. Glucose-6-phosphate dehydrogenase deficiency. The Lancet 371, 64-74, doi:http://dx.doi.org/10.1016/S0140-6736(08)60073-2 (2008).

10 Chen, L. et al. NADPH production by the oxidative pentose-phosphate pathway supports folate metabolism. Nature Metabolism 1, 404-415, doi:10.1038/s42255-019-0043-x (2019).

11 Hamilton, N. M. et al. Novel steroid inhibitors of glucose 6-phosphate dehydrogenase. Journal of medicinal chemistry 55, 4431-4445, doi:10.1021/jm300317k (2012).

12 Preuss, J. et al. Identification and Characterization of Novel Human Glucose-6-Phosphate Dehydrogenase Inhibitors. Journal of biomolecular screening 18, 286-297, doi:10.1177/1087057112462131 (2012).

13 Mele, L. et al. A new inhibitor of glucose-6-phosphate dehydrogenase blocks pentose phosphate pathway and suppresses malignant proliferation and metastasis in vivo. Cell Death Dis 9, 572, doi:10.1038/s41419-018-0635-5 (2018).

14 Marks, P. A. & Banks, J. INHIBITION OF MAMMALIAN GLUCOSE-6-PHOSPHATE DEHYDROGENASE BY STEROIDS. Proceedings of the National Academy of Sciences of the United States of America 46, 447-452 (1960).

15 Di Monaco, M. et al. Role of glucose-6-phosphate dehydrogenase inhibition in the antiproliferative effects of dehydroepiandrosterone on human breast cancer cells. British journal of cancer 75, 589-592 (1997).

16 Pashko, L. L., Lewbart, M. L. & Schwartz, A. G. Inhibition of 12-O-tetradecanoylphorbol-13-acetate-promoted skin tumor formation in mice by 16α-fluoro-5-androsten-17-one and its reversal by deoxyribonucleosides. Carcinogenesis 12, 2189-2192, doi:10.1093/carcin/12.11.2189 (1991).

17 Girón, R. A., Montaño, L. F., Escobar, M. L. & Lopez-Marure, R. Dehydroepiandrosterone inhibits the proliferation and induces the death of HPV-positive and HPV-negative cervical cancer cells through an androgen- and estrogen-receptor independent mechanism. The FEBS journal 276, 5598-5609, doi:doi:10.1111/j.1742-4658.2009.07253.x (2009).

18 Ho, H. Y., Cheng, M. L., Chiu, H. Y., Weng, S. F. & Chiu, D. T. Dehydroepiandrosterone induces growth arrest of hepatoma cells via alteration of mitochondrial gene expression and function. Int J Oncol 33, 969-977 (2008).

19 Pacold, M. E. et al. A PHGDH inhibitor reveals coordination of serine synthesis and one-carbon unit fate. Nature Chemical Biology 12, 452, doi:10.1038/nchembio.2070; https://www.nature.com/articles/nchembio.2070#supplementary-information (2016).

20 Mullarky, E. et al. Identification of a small molecule inhibitor of 3-phosphoglycerate dehydrogenase to target serine biosynthesis in cancers. Proceedings of the National Academy of Sciences 113, 1778, doi:10.1073/pnas.1521548113 (2016).

21 Ducker, G. S. et al. Human SHMT inhibitors reveal defective glycine import as a targetable metabolic vulnerability of diffuse large B-cell lymphoma. Proceedings of the National Academy of Sciences of the United States of America 114, 11404-11409, doi:10.1073/pnas.1706617114 (2017).

22 Lu, W., Wang, L., Chen, L., Hui, S. & Rabinowitz, J. D. Extraction and Quantitation of Nicotinamide Adenine Dinucleotide Redox Cofactors. Antioxidants & redox signaling 28, 167-179, doi:10.1089/ars.2017.7014 (2018).

23 Gordon, G., Mackow, M. C. & Levy, H. R. On the mechanism of interaction of steroids with human glucose 6-phosphate dehydrogenase. Arch Biochem Biophys 318, 25-29, doi:10.1006/abbi.1995.1199 (1995).

24 Miclet, E. et al. NMR Spectroscopic Analysis of the First Two Steps of the Pentose-Phosphate Pathway Elucidates the Role of 6-Phosphogluconolactonase. Journal of Biological Chemistry 276, 34840-34846, doi:10.1074/jbc.M105174200 (2001).

25 Zhang, Z., Chen, L., Liu, L., Su, X. & Rabinowitz, J. D. Chemical Basis for Deuterium Labeling of Fat and NADPH. Journal of the American Chemical Society 139, 14368-14371, doi:10.1021/jacs.7b08012 (2017).

26 Mele, L. et al. A new inhibitor of glucose-6-phosphate dehydrogenase blocks pentose phosphate pathway and suppresses malignant proliferation and metastasis in vivo. Cell Death & Disease 9, 572, doi:10.1038/s41419-018-0635-5 (2018).

27 Mercaldi, G. F., Ranzani, A. T. & Cordeiro, A. T. Discovery of new uncompetitive inhibitors of glucose-6-phosphate dehydrogenase. Journal of biomolecular screening 19, 1362-1371, doi:10.1177/1087057114546896 (2014).

28 Sena, L. A. et al. Mitochondria are required for antigen-specific T cell activation through reactive oxygen species signaling. Immunity 38, 225-236, doi:10.1016/j.immuni.2012.10.020 (2013).

29 Padgett, L. E. & Tse, H. M. NADPH Oxidase-Derived Superoxide Provides a Third Signal for CD4 T Cell Effector Responses. Journal of immunology (Baltimore, Md. : 1950) 197, 1733-1742, doi:10.4049/jimmunol.1502581 (2016).

30 Mak, T. W. et al. Glutathione Primes T Cell Metabolism for Inflammation. Immunity 46, 675-689, doi:10.1016/j .immuni.2017.03.019 (2017).

31 Goldfarb, A. H., McIntosh, M. K. & Boyer, B. T. Vitamin E attenuates myocardial oxidative stress induced by DHEA in rested and exercised rats. Journal of Applied Physiology 80, 486-490, doi:10.1152/jappl.1996.80.2.486 (1996).

32 Fan, J. et al. Quantitative flux analysis reveals folate-dependent NADPH production. Nature 510, 298-302, doi:10.1038/nature13236 (2014).

33 Morelli, A., Benatti, U., Gaetani, G. F. & De Flora, A. Biochemical mechanisms of glucose-6-phosphate dehydrogenase deficiency. Proceedings of the National Academy of Sciences of the United States of America 75, 1979-1983, doi:10.1073/pnas.75.4.1979 (1978).

34 Marks, P. A., Johnson, A. B. & Hirschberg, E. EFFECT OF AGE ON THE ENZYME ACTIVITY IN ERYTHROCYTES. Proceedings of the National Academy of Sciences of the United States of America 44, 529-536, doi:10.1073/pnas.44.6.529 (1958).

35 Cunningham, A. D., Colavin, A., Huang, K. C. & Mochly-Rosen, D. Coupling between Protein Stability and Catalytic Activity Determines Pathogenicity of G6PD Variants. Cell reports 18, 2592-2599, doi:10.1016/j.celrep.2017.02.048.

36 Morellini, M., Colonna-Romano, S., Meloni, T., Battistuzzi, G. & Gandini, E. Glucose-6-phosphate dehydrogenase of leukocyte subpopulations in normal and enzyme deficient individuals. Haematologica 70, 390-395 (1985).

37 Ardati, K. O., Bajakian, K. M. & Tabbara, K. S. Effect of Glucose-6-Phosphate Dehydrogenase Deficiency on Neutrophil Function. Acta Haematologica 97, 211-215, doi:10.1159/000203685 (1997).

38 Vives Corrons, J. L. et al. Severe-glucose-6-phosphate dehydrogenase (G6PD) deficiency associated with chronic hemolytic anemia, granulocyte dysfunction, and increased susceptibility to infections: description of a new molecular variant (G6PD Barcelona). Blood 59, 428-434 (1982).

39 Macintyre, A. N. et al. The glucose transporter Glut1 is selectively essential for CD4 T cell activation and effector function. Cell metabolism 20, 61-72, doi:10.1016/j.cmet.2014.05.004 (2014).

40 Ho, P. C. et al. Phosphoenolpyruvate Is a Metabolic Checkpoint of Anti-tumor T Cell Responses. Cell 162, 1217-1228, doi:10.1016/j.cell.2015.08.012 (2015).

41 Cham, C. M. & Gajewski, T. F. Glucose availability regulates IFN-gamma production and p70S6 kinase activation in CD8+ effector T cells. Journal of immunology (Baltimore, Md. : 1950) 174, 4670-4677, doi:10.4049/jimmunol.174.8.4670 (2005).

42 Shi, L. Z. et al. HIF1α-dependent glycolytic pathway orchestrates a metabolic checkpoint for the differentiation of T_{H}17 and T_{reg} cells. The Journal of experimental medicine 208, 1367-1376, doi:10.1084/jem.20110278 (2011).

43 Chang, C. H. et al. Posttranscriptional control of T cell effector function by aerobic glycolysis. Cell 153, 1239-1251, doi:10.1016/j.cell.2013.05.016 (2013).

44 Wang, R. et al. The transcription factor Myc controls metabolic reprogramming upon T lymphocyte activation. Immunity 35, 871-882, doi:10.1016/j.immuni.2011.09.021 (2011).

45 Katz, J. & Wood, H. G. The Use of C14O2 Yields from Glucose-1- and -6-C14 for the Evaluation of the Pathways of Glucose Metabolism. Journal of Biological Chemistry 238, 517-523 (1963).

46 Lu, W. et al. Metabolomic analysis via reversed-phase ion-pairing liquid chromatography coupled to a stand alone orbitrap mass spectrometer. Analytical chemistry 82, 3212-3221, doi:10.1021/ac902837x (2010).

47 Su, X., Lu, W. & Rabinowitz, J. D. Metabolite Spectral Accuracy on Orbitraps. Analytical chemistry 89, 5940-5948, doi:10.1021/acs.analchem.7b00396 (2017).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A compound having the following structural formula: 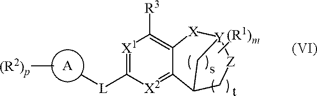
or a pharmaceutically acceptable salt thereof, wherein:
Ring A is (C₅-C₁₅)heteroaryl or (C₆-C₁₅)aryl;
L is —N(R¹⁰)(CR¹¹R¹²)_{q}, —O—(CR¹¹R¹²)_{q}, —C(O)O—, —C(O)N(R¹⁰)—, —S(O)₂N(R¹⁰)—, N(R¹⁰)C(O)N(R¹⁰)— or —C(R¹¹)(R¹²)—;
each R¹⁰, R¹¹ and R¹² is independently H or (C₁-C₆)alkyl;
q is 0 or 1;
X¹ is —N— or —C(R²¹)—;
R²¹ is H, halo, hydroxy, cyano, (C₁-C₆)alkyl, (C₁-C₆)haloalkyl, (C₁-C₆)alkoxy or (C₁-C₆)haloalkoxy;
X² is —N— or —C(R²²)—;
R²² is H, halo, hydroxy, cyano, (C₁-C₆)alkyl, (C₁-C₆)haloalkyl, (C₁-C₆)alkoxy or (C₁-C₆)haloalkoxy;
X is —C(O)—, —C(R¹⁶)—, —C(H)(OR¹³)—, —S(O)₂—, —C(NOR¹³)—, —C(F)₂—, —C(═C(CN)₂), —C(═C(H)(CN))— or —C(H)(C(H)(CN)₂)—;
R¹³ is H or (C₁-C₆)alkyl;
Y is —C(H)₂—, —C(R¹⁷)— or —N(H)— when s is 0, and —C(H)— when s is 1 or 2;
R¹⁶ and R¹⁷, taken together with X and Y, form a (C₅-C₆)heteroaryl optionally substituted with one, two or three substituents independently selected from halo, hydroxy, cyano, (C₁-C₆)alkyl, (C₁-C₆)haloalkyl, (C₁-C₆)alkoxy or (C₁-C₆)haloalkoxy;
Z is —C(H)₂— or —O—, or Z is absent;
R¹, for each occurrence, is independently halo, hydroxy, cyano, (C₁-C₆)alkyl, (C₁-C₆)haloalkyl, (C₁-C₆)alkoxy or (C₁-C₆)haloalkoxy;
R², for each occurrence, is independently halo, hydroxy, cyano, nitro, —N(R¹⁴)C(O)N(R¹⁴)(R¹⁵), —N(R¹⁴)(R¹⁵), —NR¹⁴C(O)R¹⁵, —S(O)₂N(R¹⁴)(R¹⁵), —S(O)₂R¹⁸, —C(O)N(R¹⁴)(R¹⁵), —C(O)OR¹⁴, —C(O)R¹⁸, —OC(O)R¹⁸, (C₁-C₆)alkyl, (C₁-C₆)alkenyl, (C₁-C₆)alkynyl, (C₁-C₆)haloalkyl, (C₁-C₆)hydroxyalkyl, cyano(C₁-C₆)alkyl, (C₆-C₁₅)ar(C₁-C₆)alkyl, (C₃-C₁₂)cycloalkyl, (C₃-C₁₂)heterocycloalkyl, —OR¹⁸, —SR¹⁸, (C₆-C₁₅)aryl, (C₅-C₁₅)heteroaryl or B(OR¹⁹)2;
each R¹⁴ and R¹⁵ is independently H or (C₁-C₆)alkyl;
each R¹⁸ is independently (C₁-C₁₅)alkyl, (C₁-C₁₅)alkenyl, (C₁-C₁₅)alkynyl, (C₁-C₁₅)haloalkyl, (C₁-C₁₅)hydroxyalkyl, (C₁-C₆)alkoxy(C₁-C₁₅)alkyl, amino(C₁-C₁₅)alkyl, (C₃-C₁₂)cycloalkyl, (C₃-C₁₂)heterocyclyl, (C₆-C₁₅)aryl, (C₅-C₁₅)heteroaryl, (C₃-C₁₂)cycloalkyl(C₁-C₁₅)alkyl, (C₃-C₁₂)heterocyclyl(C₁-C₁₅)alkyl, (C₆-C₁₅)ar(C₁-C₁₅)alkyl, (C₅-C₁₅)heteroaryl(C₁-C₁₅)alkyl, wherein each cycloalkyl, heterocyclyl, aryl and heteroaryl is optionally substituted with one or more R²⁰;
each R¹⁹ is independently H or (C₁-C₆)alkyl, or two R¹⁹ attached to oxygens attached to the same B, taken together with their intervening atoms, form a (C₅-C₈)heterocyclyl optionally and independently substituted with one or more (C₁-C₆)alkyl;
each R²⁰ is independently halo, hydroxy, cyano, (C₁-C₆)alkyl, (C₁-C₆)haloalkyl, (C₁-C₆)hydroxyalkyl, —C(H)₂C≡C, (C₁-C₆)alkoxy, (C₁-C₆)haloalkoxy, —NH₂ or —C(O)O(C₁-C₆)alkyl;
R³ is H, halo, hydroxy, cyano, (C₁-C₆)alkyl, (C₁-C₆)haloalkyl, (C₁-C₆)alkoxy or (C₁-C₆)haloalkoxy;
m is 0, 1, 2, 3 or 4;
p is 0, 1, 2, 3 or 4;
s is 0, 1 or 2; and
t is 0, 1 or 2,
provided that when: X¹ and X² are both —N—; X is —C(O)—; Y is —C(H)₂—; and (i) Z is —C(H)₂— and t is 0, or (ii) Z is absent and t is 1, then Ring A is not phenyl.

2. The compound of claim 1, having the following structural formula: 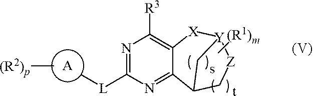
or a pharmaceutically acceptable salt thereof.

3. The compound of claim 1, wherein s is 0 and t is 0 or 1.

4. The compound of claim 1, having the following structural formula: 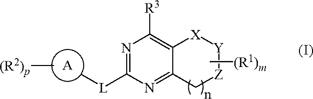
or a pharmaceutically acceptable salt thereof, wherein n is 0, 1, 2 or 3, provided that when X is —C(O)—, Y is —C(H)
₂—, Z is —C(H)₂— and n is 1, Ring A is not phenyl.

5. The compound of claim 1, wherein R², for each occurrence, is independently halo, hydroxy, cyano, (C₁-C₆)alkyl, (C₁-C₆)alkenyl, (C₁-C₆)alkynyl, (C₁-C₆)haloalkyl, —OR¹⁸, —SR¹⁸, (C₆-C₁₈)aryl or (C₅-C₁₅)heteroaryl.

6. The compound of claim 1, wherein each R¹⁸ is independently (C₁-C₆)alkyl, (C₁-C₆)alkenyl, (C₁-C₆)alkynyl, (C₁-C₆)haloalkyl, (C₁-C₆)hydroxyalkyl, (C₁-C₆)alkoxy(C₁-C₆)alkyl, amino(C₁-C₆)alkyl, (C₃-C₁₂)cycloalkyl, (C₃-C₁₂)heterocyclyl, (C₆-C₁₅)aryl, (C₅-C₁₅)heteroaryl, (C₃-C₁₂)cycloalkyl(C₁-C₆)alkyl, (C₃-C₁₂)heterocyclyl(C₁-C₆)alkyl, (C₆-C₁₅)ar(C₁-C₆)alkyl, (C₅-C₁₅)heteroaryl(C₁-C₆)alkyl, wherein each cycloalkyl, heterocyclyl, aryl and heteroaryl is optionally substituted with one or more R²⁰.

7. The compound of claim 6, wherein each R¹⁸ is independently (C₁-C₆)alkyl, (C₁-C₆)alkenyl, (C₁-C₆)alkynyl, (C₁-C₆)haloalkyl, (C₃-C₁₂)cycloalkyl, (C₃-C₁₂)heterocyclyl, (C₆-C₁₅)aryl, (C₅-C₁₅)heteroaryl, (C₃-C₁₂)cycloalkyl(C₁)alkyl, (C₃-C₁₂)heterocyclyl(C₁)alkyl, (C₆-C₁₅)ar(C₁)alkyl or (C₅-C₁₅)heteroaryl(C₁)alkyl, wherein each cycloalkyl, heterocyclyl, aryl and heteroaryl is optionally substituted with one or more R²⁰.

8. The compound of claim 1, wherein each R²⁰ is independently halo, hydroxy, cyano, (C₁-C₆)alkyl, (C₁-C₆)haloalkyl, (C₁-C₆)hydroxyalkyl, —C(H)₂C≡C, (C₁-C₆)alkoxy or (C₁-C₆)haloalkoxy.

9. The compound of claim 1, wherein the (C₅-C₆)heteroaryl formed by R¹⁶ and R¹⁷, taken together with X and Y, is an isoxazolyl, pyrazolyl, thienyl or pyridinyl optionally substituted with one, two or three substituents independently selected from halo, hydroxy, cyano, (C₁-C₆)alkyl, (C₁-C₆)haloalkyl, (C₁-C₆)alkoxy or (C₁-C₆)haloalkoxy.

10. The compound of claim 4, wherein:
Ring A is (C₅-C₁₅)heteroaryl or (C₆-C₁₅)aryl;
L is —N(R¹⁰)(CR¹¹R¹²)_{q}—, —O—(CR¹¹R¹²)_{q}—, —C(O)O—, —C(O)N(R¹⁰)—, —S(O)₂N(R¹⁰)—, N(R¹⁰)C(O)N(R¹⁰)— or —C(R¹¹)(R¹²)—;
each R¹⁰, R¹¹ and R¹² is independently H or (C₁-C₆)alkyl;
q is 0 or 1;
X is —C(O)—, —C(H)(OR¹³)—, —S(O)₂— or —C(NOR¹³)—;
R¹³ is H or (C₁-C₆)alkyl;
Y is —C(H)₂— or —N(H)—;
Z is —C(H)₂— or —O—;
R¹, for each occurrence, is independently halo, hydroxy, cyano, (C₁-C₆)alkyl, (C₁-C₆)haloalkyl, (C₁-C₆)alkoxy or (C₁-C₆)haloalkoxy;
R², for each occurrence, is independently halo, hydroxy, cyano, —N(R¹⁴)C(O)N(R¹⁴)(R¹⁵), -N(R¹⁴)(R¹⁵), —C(O)N(R¹⁴)(R¹⁵), —C(O)OR¹⁴, (C₁-C₆)alkyl, (C₁-C₆)alkenyl, (C₁-C₆)alkynyl, (C₁-C₆)haloalkyl, (C₁-C₆)hydroxyalkyl, (C₃-C₁₂)cycloalkyl, (C₃-C₁₂)heterocycloalkyl, (C₁-C₆)alkoxy, (C₁-C₆)haloalkoxy, (C₆-C₁₅)aryloxy, (C₅-C₁₅)heteroaryloxy, (C₆-C₁₅)ar(C₁-C₆)alkoxy, (C₅-C₁₅)heteroaryl(C₁-C₆)alkoxy, (C₆-C₁₅)aryl or (C₅-C₁₅)heteroaryl;
each R¹⁴ and R¹⁵ is independently H or (C₁-C₆)alkyl;
R³ is H, halo, hydroxy, cyano, (C₁-C₆)alkyl, (C₁-C₆)haloalkyl, (C₁-C₆)alkoxy or (C₁-C₆)haloalkoxy;
n is 0, 1, 2 or 3;
m is 0, 1, 2, 3 or 4; and
p is 0, 1, 2, 3 or 4.

11. The compound of claim 1, Ring A is (C₅-C₁₅)heteroaryl.

12. The compound of claim 11, wherein Ring A is thienyl, pyrrolyl, pyridinyl, isoxazolyl, indazolyl, indolyl, benzofuranyl, benzthiazolyl or benzimidazolyl.

13. The compound of claim 12, wherein Ring A is thienyl.

14. The compound of claim 1, wherein Ring A is (C₆-C₁₅)aryl.

15. The compound of claim 14, wherein Ring A is phenyl.

16. The compound of claim 1, wherein Ring A is phenyl, thienyl or pyrrolyl.

17. The compound of claim 1, wherein L is —N(H)—.

18. The compound of claim 1, wherein X is —C(O)—.

19. The compound of claim 1, wherein Y is —C(H)₂—.

20. The compound of claim 1, wherein Z is —C(H)₂—.

21. The compound of claim 1, wherein R³ is H.

22. The compound of claim 4,wherein n is 2.

23. The compound of claim 4, wherein n is 1.

24. The compound of claim 1, wherein m is 0, 1 or 2.

25. The compound of claim 1, wherein p is 0, 1 or 2.

26. The compound of claim 25, wherein p is 1.

27. The compound of claim 25, wherein p is 2.

28. The compound of claim 25, wherein p is 0.

29. The compound of claim 1, wherein p is 1 or 2, and each occurrence of R² is at the position meta to variable L.

30. The compound of claim 1, wherein Ring A is phenyl; p is 2; and R², for one occurrence, is —OR¹⁸, and for a second occurrence, is selected from halo, hydroxy, cyano or (C₁-C₆)alkyl, wherein each R² is meta to variable L.

31. The compound of claim 4, having the following structural formula: 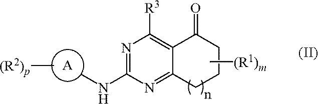
or a pharmaceutically acceptable salt thereof.

32. The compound of claim 4, having the following structural formula: 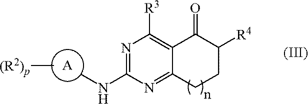
or a pharmaceutically acceptable salt thereof, wherein R
⁴ is hydrogen, halo, hydroxy, cyano, (C₁-C₆)alkyl, (C₁-C₆)haloalkyl, (C₁-C₆)alkoxy or (C₁-C₆)haloalkoxy.

33. The compound of claim 32, having the following structural formula: 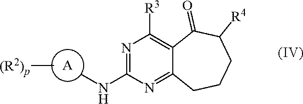
or a pharmaceutically acceptable salt thereof.

34. The compound of claim 32, wherein R⁴ is hydrogen, halo, hydroxy, (C₁-C₆)alkyl or (C₁-C₆)haloalkyl.

35. The compound of claim 34, wherein R⁴ is hydrogen.

36. A compound having a structural formula in Table 1 or Table 1A, or a pharmaceutically acceptable salt thereof.

37. A composition, comprising a compound of claim 1, or a pharmaceutically acceptable salt thereof, and a pharmaceutically acceptable carrier.

38. A method of treating a glucose-6-phosphate dehydrogenase (G6PD)-mediated disease or condition in a subject in need thereof, comprising administering to the subject a therapeutically effective amount of a compound having the following structural formula: 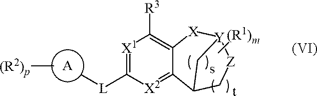
or a pharmaceutically acceptable salt thereof, wherein:
Ring A is (C₅-C₁₅)heteroaryl or (C₆-C₁₅)aryl;
L is —N(R¹⁰)(CR¹¹R¹²)_{q}, —O—(CR¹¹R¹²)_{q}, —C(O)O—, —C(O)N(R¹⁰)—, —S(O)₂N(R¹⁰)—, N(R¹⁰)C(O)N(R¹⁰)— or —C(R¹¹)(R¹²)—;
each R¹⁰, R¹¹ and R¹² is independently H or (C₁-C₆)alkyl;
q is 0 or 1;
X¹ is —N— or —C(R²¹)—;
R²¹ is H, halo, hydroxy, cyano, (C₁-C₆)alkyl, (C₁-C₆)haloalkyl, (C₁-C₆)alkoxy or (C₁-C₆)haloalkoxy;
X² is —N— or —C(R²²)—;
R²² is H, halo, hydroxy, cyano, (C₁-C₆)alkyl, (C₁-C₆)haloalkyl, (C₁-C₆)alkoxy or (C₁-C₆)haloalkoxy;
X is —C(O)—, —C(R¹⁶)—, —C(H)(OR¹³)—, —S(O)₂—, —C(NOR¹³)—, —C(F)₂—, —C(═C(CN)₂), —C(═C(H)(CN))— or —C(H)(C(H)(CN)₂)—;
R¹³ is H or (C₁-C₆)alkyl;
Y is —C(H)₂—, —C(R¹⁷)— or —N(H)— when s is 0, and —C(H)— when s is 1 or 2;
R¹⁶ and R¹⁷, taken together with X and Y, form a (C₅-C₆)heteroaryl optionally substituted with one, two or three substituents independently selected from halo, hydroxy, cyano, (C₁-C₆)alkyl, (C₁-C₆)haloalkyl, (C₁-C₆)alkoxy or (C₁-C₆)haloalkoxy;
Z is —C(H)₂— or —O—, or Z is absent;
R¹, for each occurrence, is independently halo, hydroxy, cyano, (C₁-C₆)alkyl, (C₁-C₆)haloalkyl, (C₁-C₆)alkoxy or (C₁-C₆)haloalkoxy;
R², for each occurrence, is independently halo, hydroxy, cyano, nitro, —N(R¹⁴)C(O)N(R¹⁴)(R¹⁵), —N(R¹⁴)(R¹⁵), —NR¹⁴C(O)R¹⁵, —S(O)₂N(R¹⁴)(R¹⁵), —S(O)₂R¹⁸, —C(O)N(R¹⁴)(R¹⁵), —C(O)OR¹⁴, —C(O)R¹⁸, —OC(O)R¹⁸, (C₁-C₆)alkyl, (C₁-C₆)alkenyl, (C₁-C₆)alkynyl, (C₁-C₆)haloalkyl, (C₁-C₆)hydroxyalkyl, cyano(C₁-C₆)alkyl, (C₆-C₁₅)ar(C₁-C₆)alkyl, (C₃-C₁₂)cycloalkyl, (C₃-C₁₂)heterocycloalkyl, —OR¹⁸, —SR¹⁸, (C₆-C₁₅)aryl, (C₅-C₁₅)heteroaryl or B(OR¹⁹)₂;
each R¹⁴ and R¹⁵ is independently H or (C₁-C₆)alkyl;
each R¹⁸ is independently (C₁-C₁₅)alkyl, (C₁-C₁₅)alkenyl, (C₁-C₁₅)alkynyl, (C₁-C₁₅)haloalkyl, (C₁-C₁₅)hydroxyalkyl, (C₁-C₆)alkoxy(C₁-C₁₅)alkyl, amino(C₁-C₁₅)alkyl, (C₃-C₁₂)cycloalkyl, (C₃-C₁₂)heterocyclyl, (C₆-C₁₅)aryl, (C₅-C₁₅)heteroaryl, (C₃-C₁₂)cycloalkyl(C₁-C₁₅)alkyl, (C₃-C₁₂)heterocyclyl(C₁-C₁₅)alkyl, (C₆-C₁₅)ar(C₁-C₁₅)alkyl, (C₅-C₁₅)heteroaryl(C₁-C₁₅)alkyl, wherein each cycloalkyl, heterocyclyl, aryl and heteroaryl is optionally substituted with one or more R²⁰;
each R¹⁹ is independently H or (C₁-C₆)alkyl, or two R¹⁹ attached to oxygens attached to the same B, taken together with their intervening atoms, form a (C₅-C₈)heterocyclyl optionally and independently substituted with one or more (C₁-C₆)alkyl;
each R²⁰ is independently halo, hydroxy, cyano, (C₁-C₆)alkyl, (C₁-C₆)haloalkyl, (C₁-C₆)hydroxyalkyl, —C(H)₂C≡C, (C₁-C₆)alkoxy, (C₁-C₆)haloalkoxy, —NH₂ or C(O)O(C₁-C₆)alkyl;
R³ is H, halo, hydroxy, cyano, (C₁-C₆)alkyl, (C₁-C₆)haloalkyl, (C₁-C₆)alkoxy or (C₁-C₆)haloalkoxy;
m is 0, 1, 2, 3 or 4;
p is 0, 1, 2, 3 or 4;
s is 0, 1 or 2; and
t is 0, 1 or 2.

39. A method of treating cancer, malaria, an autoimmune disease, an inflammatory disease or condition or asthma in a subject in need thereof, comprising administering to the subject a therapeutically effective amount of a compound having the following structural formula: 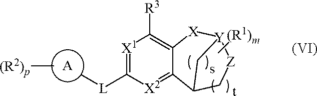
or a pharmaceutically acceptable salt thereof, wherein:
Ring A is (C₅-C₁₅)heteroaryl or (C₆-C₁₅)aryl;
L is —N(R¹⁰)(CR¹¹R¹²)_{q}, —O—(CR¹¹R¹²)_{q}, —C(O)O—, —C(O)N(R¹⁰)—, —S(O)₂N(R¹⁰)—, N(R¹⁰)C(O)N(R¹⁰)— or —C(R¹¹)(R¹²)—;
each R¹⁰, R¹¹ and R¹² is independently H or (C₁-C₆)alkyl;
q is 0 or 1;
X¹ is —N— or —C(R²¹)—;
R²¹ is H, halo, hydroxy, cyano, (C₁-C₆)alkyl, (C₁-C₆)haloalkyl, (C₁-C₆)alkoxy or (C₁-C₆)haloalkoxy;
X² is —N— or —C(R²²)—;
R²² is H, halo, hydroxy, cyano, (C₁-C₆)alkyl, (C₁-C₆)haloalkyl, (C₁-C₆)alkoxy or (C₁-C₆)haloalkoxy;
X is —C(O)—, —C(R¹⁶)—, —C(H)(OR¹³)—, —S(O)₂—, —C(NOR¹³)—, —C(F)₂—, —C(═C(CN)₂), —C(═C(H)(CN))— or —C(H)(C(H)(CN)₂)—;
R¹³ is H or (C₁-C₆)alkyl;
Y is —C(H)₂—, —C(R¹⁷)— or —N(H)— when s is 0, and —C(H)— when s is 1 or 2;
R¹⁶ and R¹⁷, taken together with X and Y, form a (C₅-C₆)heteroaryl optionally substituted with one, two or three substituents independently selected from halo, hydroxy, cyano, (C₁-C₆)alkyl, (C₁-C₆)haloalkyl, (C₁-C₆)alkoxy or (C₁-C₆)haloalkoxy;
Z is —C(H)₂— or —O—, or Z is absent;
R¹, for each occurrence, is independently halo, hydroxy, cyano, (C₁-C₆)alkyl, (C₁-C₆)haloalkyl, (C₁-C₆)alkoxy or (C₁-C₆)haloalkoxy;
R², for each occurrence, is independently halo, hydroxy, cyano, nitro, —N(R¹⁴)C(O)N(R¹⁴)(R¹⁵), —N(R¹⁴)(R¹⁵), —NR¹⁴C(O)R¹⁵, —S(O)₂N(R¹⁴)(R^{1S}), —S(O)₂R¹⁸, —C(O)N(R¹⁴)(R¹⁵), —C(O)OR¹⁴, —C(O)R¹⁸, —OC(O)R¹⁸, (C₁-C₆)alkyl, (C₁-C₆)alkenyl, (C₁-C₆)alkynyl, (C₁-C₆)haloalkyl, (C₁-C₆)hydroxyalkyl, cyano(C₁-C₆)alkyl, (C₆-C₁₅)ar(C₁-C₆)alkyl, (C₃-C₁₂)cycloalkyl, (C₃-C₁₂)heterocycloalkyl, —OR¹⁸, —SR¹⁸, (C₆-C₁₅)aryl, (C₅-C₁₅)heteroaryl or B(OR¹⁹)2;
each R¹⁴ and R¹⁵ is independently H or (C₁-C₆)alkyl;
each R¹⁸ is independently (C₁-C₁₅)alkyl, (C₁-C₁₅)alkenyl, (C₁-C₁₅)alkynyl, (C₁-C₁₅)haloalkyl, (C₁-C₁₅)hydroxyalkyl, (C₁-C₆)alkoxy(C₁-C₁₅)alkyl, amino(C₁-C₁₅)alkyl, (C₃-C₁₂)cycloalkyl, (C₃-C₁₂)heterocyclyl, (C₆-C₁₅)aryl, (C₅-C₁₅)heteroaryl, (C₃-C₁₂)cycloalkyl(C₁-C₁₅)alkyl, (C₃-C₁₂)heterocyclyl(C₁-C₁₅)alkyl, (C₆-C₁₅)ar(C₁-C₁₅)alkyl, (C₅-C₁₅)heteroaryl(C₁-C₁₅)alkyl, wherein each cycloalkyl, heterocyclyl, aryl and heteroaryl is optionally substituted with one or more R²⁰;
each R¹⁹ is independently H or (C₁-C₆)alkyl, or two R¹⁹ attached to oxygens attached to the same B, taken together with their intervening atoms, form a (C₅-C₈)heterocyclyl optionally and independently substituted with one or more (C₁-C₆)alkyl;
each R²⁰ is independently halo, hydroxy, cyano, (C₁-C₆)alkyl, (C₁-C₆)haloalkyl, (C₁-C₆)hydroxyalkyl, —C(H)₂C≡C, (C₁-C₆)alkoxy, (C₁-C₆)haloalkoxy, —NH₂ or C(O)O(C₁-C₆)alkyl;
R³ is H, halo, hydroxy, cyano, (C₁-C₆)alkyl, (C₁-C₆)haloalkyl, (C₁-C₆)alkoxy or (C₁-C₆)haloalkoxy;
m is 0, 1, 2, 3 or 4;
p is 0, 1, 2, 3 or 4;
s is 0, 1 or 2; and
t is 0, 1 or 2.

40. The method of claim 38, wherein the method is a method of treating cancer in a subject in need thereof.

41. The method of claim 40, wherein the cancer is a leukemia or lymphoma.

42. The method of claim 41, wherein the leukemia or lymphoma is a T-cell leukemia or T-cell lymphoma.

43. The method of claim 38, wherein the method is a method of treating malaria in a subject in need thereof.

44. The method of claim 38, wherein the method is a method of treating an autoimmune disease in a subject in need thereof.

45. The method of claim 38, wherein the method is a method of treating an inflammatory disease or condition in a subject in need thereof.

46. The method of claim 38, wherein the method is a method of treating asthma in a subject in need thereof.

47. The method of claim 38, further comprising administering to the subject a therapeutically effective amount of an additional therapeutic agent.
